(19) United States
(12) Patent Application Publication
(10) Pub. No.: US 2026/0293900 A1
(43) Pub. Date: Oct. 1, 2026

(54) PYRAZOLO[1,5-A]PYRIDINE DERIVATIVES

(71) Applicant: SYNGENTA CROP PROTECTION AG, Basel (CH)

(72) Inventors: Martin POULIOT, Stein (CH); Nicolas GERMAIN, Stein (CH); Stephane André Marie JEANMART, Stein (CH); Camille LE CHAPELAIN, Stein (CH)

(73) Assignee: SYNGENTA CROP PROTECTION AG, Basel (CH)

(21) Appl. No.: 19/474,569

(22) PCT Filed: Apr. 11, 2024
(86) PCT No.: PCT/EP2024/059883

§ 371 Date: Oct. 10, 2025

(30) Foreign Application Priority Data
Apr. 13, 2023 (EP) 23167795.6

(51) Int. Cl. A01N 43/90; A01P 3/00; C07D 471/04

(52) U.S. Cl. CPC A01N 43/90; A01P 3/00; C07D 471/04

(57) ABSTRACT

The current invention relates to compounds of the formula (I) wherein the substituents are as defined in claim 1, to processes and methods for preparing compounds of formula (I), to agrochemical compositions comprising compounds of formula (I) as defined in claim 1, to preparation of these compositions and to the use of the compounds or compositions in agriculture or horticulture for combating, preventing or controlling infestation of plants, harvested food crops, seeds or non-living materials by phytopathogenic microorganisms, in particular fungi.

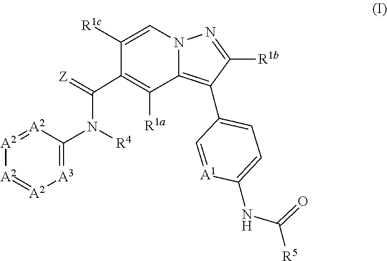

The present invention relates to microbiocidal pyrazolo[1,5-a]pyridine derivatives, e.g. as active ingredients, which have microbiocidal activity, in particular fungicidal activity, more particularly activity against oomycetes. The invention also relates to preparation of these pyrazolo[1,5-a]pyridine derivatives, to intermediates useful in the preparation of these pyrazolo[1,5-a]pyridine derivatives, to the preparation of these intermediates, to agrochemical compositions which comprise at least one of the pyrazolo[1,5-a]pyridine derivatives, to preparation of these compositions and to the use of the pyrazolo[1,5-a]pyridine derivatives or compositions in agriculture or horticulture for combating, controlling or preventing infestation of plants, harvested food crops, seeds or non-living materials by phytopathogenic microorganisms, in particular fungi, more particularly oomycetes.

It has now surprisingly been found that certain novel pyrazolo[1,5-a]pyridine derivatives have favourable fungicidal properties, in particular against oomycetes.

Therefore, in a first aspect, the present invention provides compounds of formula (I), preferably used as a fungicide,

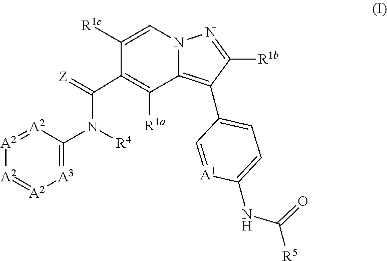

wherein Z is O or S, and preferably Z is O;
A¹ is CH or N, and preferably N;
R^{1a}, R^{1b} and R^{1c} are independently selected from hydrogen, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl;
A² are independently CR² or N, with the proviso that no more than three A² are N, preferably no more than two A² are N, preferably no more than one A² is N, and more preferably the four A² are CR²;
R² are independently selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen, hydroxy, and CN; and preferably with the proviso that at least two R² are not hydrogen;
A³ is independently CR³ or N;
R³ is selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, amino, C₁₋₆alkylamino, diC₁₋₆-alkylamino, and C₃₋₆cycloalkylamino, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, amino, C₁₋₆alkylamino, diC₁₋₆-alkylamino, and C₃₋₆cycloalkylamino groups is optionally substituted with one to three substituents independently selected from halogen, hydroxy, and CN;
R⁴ is selected from C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, C₁₋₆alkoxy, C₁₋₆alkylsulfanyl-C₁₋₆alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl, diC₁₋₆alkylaminocarbonyl-C₁₋₆alkyl, and CN, wherein each of the C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, C₁₋₆alkoxy, C₁₋₆alkylsulfanyl-C₁₋₆alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl and diC₁₋₆alkylaminocarbonyl-C₁₋₆alkyl groups is optionally substituted with one to three substituents independently selected from halogen and CN;
wherein A³ and R⁴ taken together optionally form a ring, preferably a 5-8-membered heterocycle, and more preferably a 6-membered heterocycle; and
R⁵ is selected from C₁₋₆alkyl, C₁₋₆alkoxy, C₃₋₆cycloalkyl, C₃₋₆cycloalkyl-C₁₋₆alkyl, C₁₋₆alkoxyC₁₋₆alkyl, C₁₋₆alkylamino, diC₁₋₆alkylamino, and C₁₋₆alkylC₁₋₆alkoxyamino, wherein each of said groups is optionally substituted with one to three substituents independently selected from halogen and CN;
or a salt or N-oxide thereof.

In a particular embodiment of this first aspect, the present invention can relate to the use of a compound of formula (I) as a fungicide

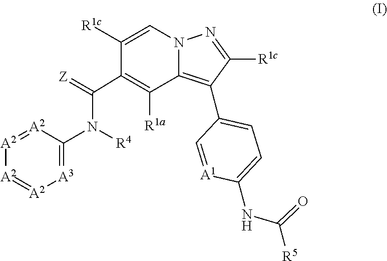

wherein Z is O or S, and preferably Z is O;
A¹ is CH or N, and preferably N;
R^{1a}, R^{1b} and R^{1c} are independently selected from hydrogen, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl; and preferably R^{1a}, R^{1b} and R^{1c} are hydrogen;
A² are independently CR² or N, with the proviso that no more than three A² are N, preferably no more than two A² are N, preferably no more than one A² is N, and more preferably the four A² are CR²;
R² are independently selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen, hydroxy, and CN; preferably R² are independently selected from hydrogen, hydroxy, halogen, CN, C₁₋₆alkyl, C₁₋₆alkoxy, C₁₋₆alkoxy-C₁₋₆alkyl, C₁₋₆alkoxycarbonyl, and C₁₋₆alkoxy-C₁₋₆alkoxy, C₁₋₆alkoxycarbonyl, C₁₋₆alkylaminocarbonyl, diC₁₋₆alkylaminocarbonyl, and C₁₋₆alkylcarbonyl, wherein each of the C₁₋₆alkyl, C₁₋₆alkoxy, C₁₋₆alkoxy-C₁₋₆alkyl, C₁₋₆alkoxycarbonyl, C₁₋₆alkoxy-C₁₋₆alkoxy, C₁₋₆alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen, hydroxy, and CN; preferably R² are independently selected from hydrogen, halogen, CN, C₁₋₆alkyl, C₁₋₆alkoxy, C₁₋₆alkoxy-C₁₋₆alkyl, C₁₋₆alkoxycarbonyl, and C₁₋₆alkoxy-C₁₋₆alkoxy, wherein each of the C₁₋₆alkyl, C₁₋₆alkoxy, C₁₋₆alkoxy-C₁₋₆alkyl, C₁₋₆alkoxycarbonyl, and C₁₋₆alkoxy-C₁₋₆alkoxy groups is optionally substituted with one to three substituents independently selected from halogen, hydroxy, and CN; and more preferably R² are independently selected from hydrogen, halogen, CN, C₁₋₆alkyl, C₁₋₆alkoxycarbonyl, and C₁₋₆alkoxy;
A³ is independently CR³ or N;
R³ is selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, amino, C₁₋₆alkylamino, diC₁₋₆-alkylamino, and C₃₋₆cycloalkylamino, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, amino, C₁₋₆alkylamino, diC₁₋₆-alkylamino, and C₃₋₆cycloalkylamino groups is optionally substituted with one to three substituents independently selected from halogen, hydroxy, and CN; and preferably R³ is hydrogen;
R⁴ is selected from C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, C₁₋₆alkoxy, C₁₋₆alkylsulfanyl-C₁₋₆alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl, diC₁₋₆alkylaminocarbonyl-C₁₋₆alkyl, and CN, wherein each of the C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, C₁₋₆alkoxy, C₁₋₆alkylsulfanyl-C₁₋₆alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl and diC₁₋₆alkylaminocarbonyl-C₁₋₆alkyl groups is optionally substituted with one to three substituents independently selected from halogen and CN;
wherein A³ and R⁴ taken together optionally form a ring, preferably a 5-8-membered heterocycle, and more preferably a 6-membered heterocycle; and
R⁵ is selected from C₁₋₆alkyl, C₁₋₆alkoxy, C₃₋₆cycloalkyl, C₃₋₆cycloalkyl-C₁₋₆alkyl, C₁₋₆alkoxyC₁₋₆alkyl, C₁₋₆alkylamino, diC₁₋₆alkylamino, and C₁₋₆alkylC₁₋₆alkoxyamino, wherein each of said groups is optionally substituted with one to three substituents independently selected from halogen and CN;
or a salt or N-oxide thereof.

In another particular embodiment of this first aspect, the present invention can relate to a compound of formula (I)

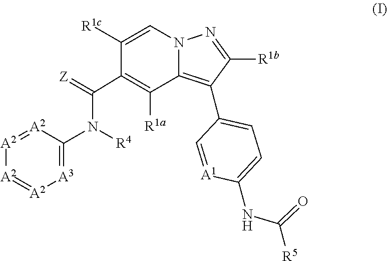

wherein Z is 0 or S, and preferably Z is O;
A¹ is CH or N, and preferably N;
R^{1a}, R^{1b} and R^{1c} are independently selected from hydrogen, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl; and preferably R^{1a}, R^{1b} and R^{1c} are hydrogen;
   A² are independently CR² or N, with the proviso that no more than three A² are N, preferably no more than two A² are N, preferably no more than one A² is N, and more preferably the four A² are CR²;
   R² are independently selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen, hydroxy, and CN;
with the proviso that at least two R² are not hydrogen, said at least two R² being preferably selected from halogen, CN, C₁₋₆alkyl, C₁₋₆alkoxy, C₁₋₆alkoxy-C₁₋₆alkyl, and C₁₋₆alkoxy-C₁₋₆alkoxy, wherein each of the C₁₋₆alkyl, C₁₋₆alkoxy, C₁₋₆alkoxy-C₁₋₆alkyl, and C₁₋₆alkoxy-C₁₋₆alkoxy groups is optionally substituted with one to three substituents independently selected from halogen, hydroxy, and CN; and more preferably selected from halogen, CN, C₁₋₆alkyl, and C₁₋₆alkoxy;
A³ is independently CR³ or N;
R³ is selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, amino, C₁₋₆alkylamino, diC₁₋₆alkylamino, and C₃₋₆cycloalkylamino, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, amino, C₁₋₆alkylamino, diC₁₋₆-alkylamino, and C₃₋₆cycloalkylamino groups is optionally substituted with one to three substituents independently selected from halogen, hydroxy, and CN; and preferably R³ is hydrogen;
R⁴ is selected from C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, C₁₋₆alkoxy, C₁₋₆alkylsulfanyl-C₁₋₆alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl, diC₁₋₆alkylaminocarbonyl-C₁₋₆alkyl, and CN, wherein each of the C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, C₁₋₆alkoxy, C₁₋₆alkylsulfanyl-C₁₋₆alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl and diC₁₋₆alkylaminocarbonyl-C₁₋₆alkyl groups is optionally substituted with one to three substituents independently selected from halogen and CN;
wherein A³ and R⁴ taken together optionally form a ring, preferably a 5-8-membered heterocycle, and more preferably a 6-membered heterocycle; and
R⁵ is selected from C₁₋₆alkyl, C₁₋₆alkoxy, C₃₋₆cycloalkyl, C₃₋₆cycloalkyl-C₁₋₆alkyl, C₁₋₆alkoxyC₁₋₆alkyl, C₁₋₆alkylamino, diC₁₋₆alkylamino, and C₁₋₆alkylC₁₋₆alkoxyamino, wherein each of said groups is optionally substituted with one to three substituents independently selected from halogen and CN;
or a salt or N-oxide thereof.

In a second aspect the present invention provides an agrochemical composition comprising a compound of formula (I), and more particularly an agrochemical composition comprising a fungicidally effective amount of a compound of formula (I). Said composition can further comprise at least one compound selected from an additional active ingredient, an appropriate formulation inert, a carrier, an adjuvant, and any mixtures thereof.

Compounds of formula (I) may be used to control phytopathogenic microorganisms. Thus, in order to control a phytopathogen a compound of formula (I), or a composition comprising a compound of formula (I) according to the invention, may be applied directly to the phytopathogen, to the locus of a phytopathogen, in particular to a plant susceptible to attack by phytopathogens, or to a propagation material of a plant.

Thus, in a third aspect the present invention provides the use of a compound of formula (I), or a composition comprising a compound of formula (I), as described herein to combat, prevent or control a phytopathogen.

In a fourth aspect the present invention provides a method of combating, preventing or controlling phytopathogens, comprising applying a compound of formula (I), or a composition comprising a compound of formula (I), as described herein to said phytopathogen, to the locus of said phytopathogen, in particular to a plant susceptible to attack by a phytopathogen, or to a propagation material of a plant. According to this fourth aspect of the invention, the method may exclude methods for the treatment of the human or animal body by surgery or therapy.

Compounds of formula (I) are particularly effective in combating, preventing or controlling phytopathogenic fungi, in particular oomycetes. Thus, in a fifth aspect the present invention provides the use of a compound of formula (I), or a composition comprising a compound of formula (I), as described herein to control phytopathogenic fungi, in particular oomycetes.

In a sixth aspect the present invention provides a method of combating, preventing or controlling phytopathogenic disease, such as phytopathogenic fungi, comprising applying a compound of formula (I), or a composition comprising a compound of formula (I), as described herein to said phytopathogenic fungi, or to the locus of said phytopathogenic fungi, in particular to a plant susceptible to attack by phytopathogenic fungi, in particular oomycetes, or to a propagation material of a plant. According to this sixth aspect of the invention, the method may exclude methods for the treatment of the human or animal body by surgery or therapy.

Where a group is indicated as being substituted, e.g. alkyl, this includes those groups that are part of other groups, e.g. the alkyl in alkylthio.

### Definitions

The term “halogen” or “halo” refers to fluorine (fluoro or F), chlorine (chloro or C₁), bromine (bromo or Br) or iodine (iodo or I), preferably fluorine, chlorine or bromine.
The term “amino” refers to a —NH₂ group.
The term “Alkyl” as used herein—in isolation or as part of a chemical group—represents straight-chain or branched hydrocarbons, preferably with 1 to 6 carbon atoms, for example methyl, ethyl, n-propyl, isopropyl, n-butyl, isobutyl, s-butyl, t-butyl, pentyl, 1-methylbutyl, 2-methylbutyl, 3-methylbutyl, 1,2-dimethylpropyl, 1,1-dimethylpropyl, 2,2-dimethylpropyl, 1-ethylpropyl, hexyl, 1-methylpentyl, 2-methylpentyl, 3-methylpentyl, 4-methylpentyl, 1,2-dimethylpropyl, 1,3-dimethylbutyl, 1,4-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl and 2-ethylbutyl. Alkyl groups with 1 to 4 carbon atoms are preferred, for example methyl, ethyl, n-propyl, isopropyl, n-butyl, isobutyl, s-butyl or t-butyl.
The term “Alkenyl”—in isolation or as part of a chemical group—represents straight-chain or branched hydrocarbons, preferably with 2 to 6 carbon atoms and at least one double bond, for example vinyl, 2-propenyl, 2-butenyl, 3-butenyl, 1-methyl-2-propenyl, 2-methyl-2-propenyl, 2-pentenyl, 3-pentenyl, 4-pentenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-2-propenyl, 1-ethyl-2-propenyl, 2-hexenyl, 3-hexenyl, 4-hexenyl, 5-hexenyl, 1-methyl-2-pentenyl, 2-methyl-2-pentenyl, 3-methyl-2-pentenyl, 4-methyl-2-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1, 1-dimethyl-2-butenyl, 1,1-dimethyl-3-butenyl, 1,2-dimethyl-2-butenyl, 1,2-dimethyl-3-butenyl, 1,3-dimethyl-2-butenyl, 2,2-dimethyl-3-butenyl, 2,3-dimethyl-2-butenyl, 2,3-dimethyl-3-butenyl, 1-ethyl-2-butenyl, 1-ethyl-3-butenyl, 2-ethyl-2-butenyl, 2-ethyl-3-butenyl, 1, 1,2-trimethyl-2-propenyl, 1-ethyl-1-methyl-2-propenyl und 1-ethyl-2-methyl-2-propenyl. Alkenyl groups with 2 to 4 carbon atoms are preferred, for example 2-propenyl, 2-butenyl or 1-methyl-2-propenyl.
The term “Alkynyl”—in isolation or as part of a chemical group—represents straight-chain or branched hydrocarbons, preferably with 2 to 6 carbon atoms and at least one triple bond, for example 2-propynyl, 2-butynyl, 3-butynyl, 1-methyl-2-propynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-4-pentynyl, 4-methyl-2-pentynyl, 1,1-dimethyl-3-butynyl, 1,2-dimethyl-3-butynyl, 2,2-dimethyl-3-butynyl, 1-ethyl-3-butynyl, 2-ethyl-3-butynyl, 1-ethyl-1-methyl-2-propynyl and 2,5-hexadiynyl. Alkynyls with 2 to 4 carbon atoms are preferred, for example ethynyl, 2-propynyl or 2-butynyl-2-propenyl.
The term “haloalkyl” refers to an alkyl radical as generally defined above substituted by one or more of the same or different halogen atoms, for examples fluoromethyl, fluoroethyl, difluoromethyl, trifluoromethyl, or 2,2,2-trifluoroethyl.
The term cyanoalkyl” refers to an alkyl radical as generally defined above substituted by one or more cyano groups.
The term “cycloalkyl”—in isolation or as part of a chemical group—represents saturated or partially unsaturated mono-, bi- or tricyclic hydrocarbons, preferably with 3 to 10 carbon atoms, for example cyclopropyl, cyclobutyl, cyclopentyl, cyclohexyl, cycloheptyl, cyclooctyl, bicyclo[2.2.1]heptyl, bicyclo[2.2.2]octyl or adamantyl. Cycloalkyls with 3, 4, 5, 6 or 7 carbon atoms are preferred, for example cyclopropyl or cyclobutyl.
The term “halocycloalkyl” refers to a cycloalkyl ring as defined above substituted by one or more of the same or different halogen atoms.
The term cyanocycloalkyl” refers to a cycloalkyl radical as generally defined above substituted by one or more cyano groups.
The term “alkoxy” refers to a radical of the formula —ORₐ wherein Rₐ is an alkyl radical as generally defined above. Examples of alkoxy include, but are not limited to methoxy, ethoxy, propoxy, iso-propoxy, and tert-butoxy. The term “alkoxyalkyl” refers to an alkyl radical (as mentioned above) substituted with said alkoxy group. Examples are methoxymethyl, methoxyethyl, ethoxymethyl and propoxymethyl.
The term “alkylsulfanyl” refers to a radical of the formula —SRₐ wherein Rₐ is an alkyl radical as generally defined above.
The term “alkylsulfinyl” refers to a radical of the formula —S(O)Rₐ wherein Rₐ is an alkyl radical as generally defined above.
The term “alkylsulfonyl” refers to a radical of the formula —S(O)₂Rₐ wherein Rₐ is an alkyl radical as generally defined above.
The term “alkylcarbonyl” refers to a radical of the formula RₐC(O)— wherein Rₐ is an alkyl radical as generally defined above.
the term “alkoxycarbonyl” refers to a radical of the formula RₐOC(O)—, wherein Rₐ is an alkyl radical as generally defined above.
The term “alkylamino” refers to a radical of the formula RₐNH— wherein Rₐ is an alkyl radical as generally defined above.
The term “cycloalkylamino” refers to a radical of the formula RₐNH— wherein Rₐ is a cycloalkyl radical as generally defined above.
The term “alkoxyamino” refers to a radical of the formula RₐNH—, wherein Rₐ is an alkoxy radical as generally defined above.
The term “alkylaminocarbonyl” refers to a radical of the formula RₐNHC(O)—wherein Rₐ is an alkyl radical as generally defined above.
Hydroxyl or hydroxy stands for a —OH group.

The term “combating”, “preventing” or “controlling”, and its inflections, within the context of the present invention, mean reducing any undesired effect, such as pathogenic and more particularly phytopathogenic, especially fungi such as oomycetes, infestation or attack of, and pathogenic damage to a plant or to a plant derived product to such a level that an improvement is demonstrated.

As used herein, the term “effective amount” refers to the amount of the compound, a salt, or N-oxide thereof, which, upon single or multiple applications provides the desired effect.

An effective amount is readily determined by the skilled person in the art, by the use of known techniques and by observing results obtained under analogous circumstances. In determining the effective amount a number of factors are considered including, but not limited to: the type of plant or derived product to be applied; the pathogen to be controlled & its lifecycle; the particular compound applied; the type of application; and other relevant circumstances.

Compounds of formula (I) which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula (I) which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

In each case, the compounds of formula (I) according to the invention are in free form, in oxidized form as an N-oxide, in covalently hydrated form, or in salt form, e.g., an agronomically usable or agrochemically acceptable salt form. N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991. The compounds of formula (I) according to the invention also include hydrates, which may be formed during salt formation.

The compounds of formula (I) according to the invention also include hydrates which may be formed during the salt formation.

In a further embodiment, there is provided a compound of formula (I) according to the present invention, wherein R^{1a}, R^{1b} and R^{1c} are independently selected from hydrogen, C₁₋₆alkyl, C₃₋₆cycloalkyl, C₁₋₆alkoxy-C₁₋₆alkyl, C₃₋₆cycloalkyl-C₁₋₄alkyl, and C₁₋₆alkoxy; and preferably R^{1a}, R^{1b} and R^{1C} are hydrogen.

In a further embodiment, there is provided a compound of formula (I) according to the present invention, wherein R³ is selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₃₋₆cycloalkyl-C₁₋₆alkyl, amino, C₁₋₆alkylamino, diC₁₋₆-alkylamino, and C₃₋₆cycloalkylamino, wherein each of the C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₃₋₆cycloalkyl-C₁₋₆alkyl, amino, C₁₋₆alkylamino, diC₁₋₆-alkylamino and C₃₋₆cycloalkylamino groups is optionally substituted with one to three substituents independently selected from halogen, hydroxy, and CN. In a preferred embodiment, R³ can be hydrogen.

In a further embodiment, there is provided a compound of formula (I) according to the present invention, wherein four A² are CR² and A³ is N.

In a further embodiment, there is provided a compound of formula (I) according to the present invention, wherein

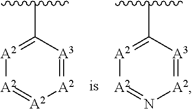

and preferably the three A² are CR² and A³ is CR³.

In a further embodiment, there is provided a compound of formula (I) according to the present invention, wherein

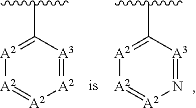

and preferably the three A² are CR² and A³ is CR³.

In a further embodiment, there is provided a compound of formula (I) according to the present invention, wherein four A² are CR² and A³ is CR³, and preferably

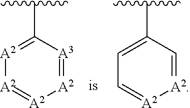

In the particular embodiment wherein

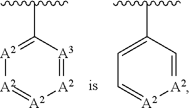

R² are as defined in the present invention; preferably R² are independently selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₃₋₆cycloalkyl-C₁₋₆alkyl, C₁₋₆alkoxycarbonyl, C₁₋₆alkylaminocarbonyl, diC₁₋₆alkylaminocarbonyl, and C₁₋₆alkylcarbonyl, wherein each of the C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₃₋₆cycloalkyl-C₁₋₆alkyl, C₁₋₆alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen, hydroxy, and CN; preferably R² are independently selected from hydrogen, halogen, CN, C₁₋₆alkoxy, C₁₋₆alkoxy-C₁₋₆alkyl, C₁₋₆alkoxycarbonyl, and C₁₋₆alkoxy-C₁₋₆alkoxy, wherein each of the C₁₋₆alkoxy, C₁₋₆alkoxy-C₁₋₆alkyl, C₁₋₆alkoxycarbonyl, and C₁₋₆alkoxy-C₁₋₆alkoxy groups is optionally substituted with one to three substituents independently selected from halogen, hydroxy, and CN; and more preferably R² are independently selected from hydrogen, halogen, CN, C₁₋₆alkyl, C₁₋₆ alkoxycarbonyl, and C₁₋₆alkoxy.

In a further embodiment, there is provided a compound of formula (I) according to the present invention, wherein R⁴ is selected from C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, and C₁₋₆alkoxy, wherein each of the C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, and C₁₋₆alkoxy groups is optionally substituted with one to three substituents independently selected from halogen and CN; preferably R⁴ is selected from C₁₋₆ alkyl, C₁₋₆alkoxy-C₁₋₆alkyl, C₃₋₆cycloalkyl, C₃₋₆cycloalkyl-C₁₋₄alkyl, and C₁₋₆alkoxy, wherein each of the C₁₋₆alkyl, C₁₋₆alkoxy-C₁₋₆alkyl, C₃₋₆cycloalkyl, C₃₋₆cycloalkyl-C₁₋₄alkyl, and C₁₋₆alkoxy groups is optionally substituted with one to three substituents independently selected from halogen and CN; and wherein A³ and R⁴ taken together optionally form a ring, more preferably a 5-8-membered heterocycle, more preferably a 6-membered heterocycle.

In a further embodiment, there is provided a compound of formula (I) according to the present invention, wherein A³ is CR³ and wherein R³ and R⁴ taken together form a ring, preferably a 5-8-membered heterocycle, preferably a 6-membered heterocycle, and more preferably one of the rings W1, W2 or W3 as described in the compounds of the formula (I) below:

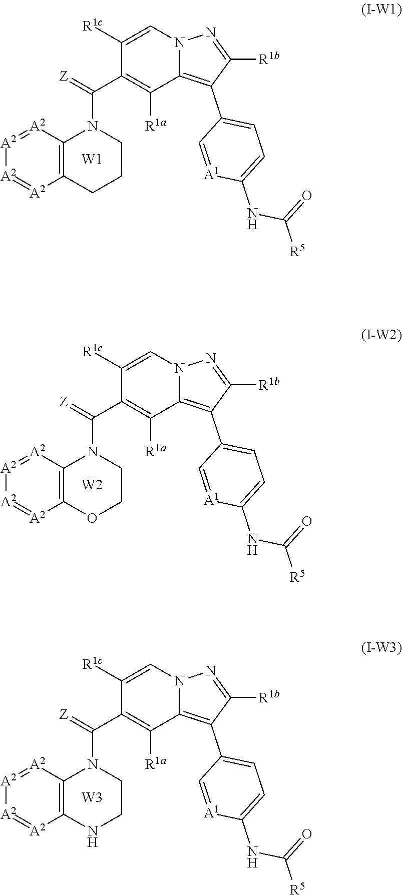

The carbon and/or the nitrogen atoms forming said ring (W1, W2 or W3) can be substituted, especially by a R^{3′} group, wherein R^{3′} is selected from hydrogen, C₁₋₆alkyl, and C₃₋₆cycloalkyl, wherein each of the C₁₋₆alkyl and C₃₋₆cycloalkyl groups is optionally substituted with one to three substituents independently selected from halogen and CN. For example, the compounds of the formula (I-W3) can be as follows:

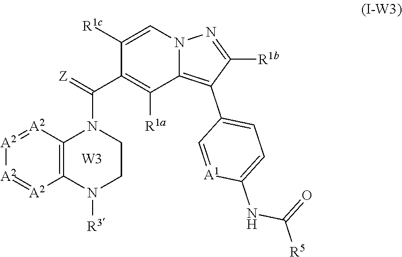

In a preferred embodiment, the compounds of the formula (I-W1), (I-W2) and (I-W3) can be as described below:

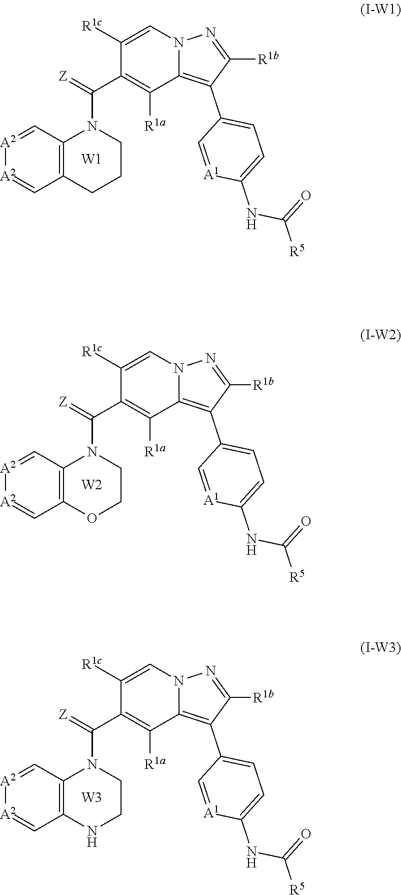

The carbon and/or the nitrogen atoms forming said ring (W1, W2 or W3) can be substituted, especially by a R^{3′} group, wherein R^{3′} is selected from hydrogen, C₁₋₆alkyl, and C₃₋₆cycloalkyl, wherein each of the C₁₋₆alkyl and C₃₋₆cycloalkyl groups is optionally substituted with one to three substituents independently selected from halogen and CN. For example, the compounds of the formula (I-W3) can be as follows:

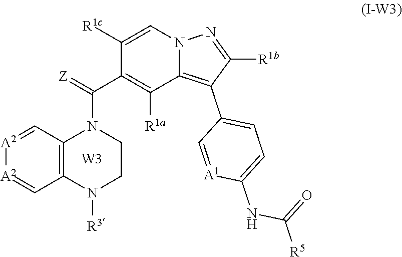

In a further embodiment, there is provided a compound of formula (I) according to the present invention, wherein R⁵ is selected from C₁₋₆alkyl, C₁₋₆alkoxy, C₃₋₆cycloalkyl, C₃₋₆cycloalkyl-C₁₋₆alkyl, and C₁₋₆alkoxyC₁₋₆alkyl, wherein each of said groups is optionally substituted with one to three substituents independently selected from halogen and CN.

In a particular embodiment, there is provided a compound of formula (I) according to the present invention, wherein
Z is O;
A¹ is N;
R^{1a} is selected from hydrogen, hydroxy, halogen, CN, C₁₋₆alkyl, C₃₋₆cycloalkyl, C₁₋₆alkoxy-C₁₋₆alkyl, C₃₋₆ cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl; and preferably R^{1a} is hydrogen;
R^{1b} is selected from hydrogen, hydroxy, halogen, CN, C₁₋₆alkyl, C₃₋₆cycloalkyl, C₁₋₆alkoxy-C₁₋₆alkyl, C₃₋₆ cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl; and preferably R^{1b} is hydrogen;
R^{1c} is selected from hydrogen, hydroxy, halogen, CN, C₁₋₆alkyl, C₃₋₆cycloalkyl, C₁₋₆alkoxy-C₁₋₆alkyl, C₃₋₆ cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl; and preferably R^{1c} is hydrogen;
the four A² are CR²; with R² being independently selected from hydrogen, hydroxy, halogen, CN, C₁₋₆ alkyl, C₁₋₆alkoxy, C₁₋₆alkoxy-C₁₋₆alkyl, and C₁₋₆alkoxy-C₁₋₆alkoxy, C₁₋₆alkoxycarbonyl, C₁₋₆alkylaminocarbonyl, diC₁₋₆alkylaminocarbonyl, and C₁₋₆alkylcarbonyl, wherein each of the C₁₋₆alkyl, C₁₋₆alkoxy, C₁₋₆alkoxy-C₁₋₆alkyl, C₁₋₆alkoxy-C₁₋₆alkoxy, C₁₋₆alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen, hydroxy, and CN; preferably R² are independently selected from hydrogen, halogen, CN, C₁₋₆alkyl, C₁₋₆alkoxy, C₁₋₆alkoxy-C₁₋₆alkyl, C₁₋₆alkoxycarbonyl, and C₁₋₆alkoxy-C₁₋₆alkoxy, wherein each of the C₁₋₆alkyl, C₁₋₆alkoxy, C₁₋₆alkoxy-C₁₋₆alkyl, C₁₋₆alkoxycarbonyl, and C₁₋₆alkoxy-C₁₋₆alkoxy groups is optionally substituted with one to three substituents independently selected from halogen, hydroxy, and CN; and more preferably R² are independently selected from hydrogen, halogen, CN, C₁₋₆alkyl, C₁₋₆alkoxycarbonyl, and C₁₋₆alkoxy; A³ is CR³ with R³ being independently selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₃₋₆cycloalkyl-C₁₋₆alkyl, amino, C₁₋₆alkylamino, diC₁₋₆-alkylamino, and C₃₋₆cycloalkylamino, wherein each of the C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₃₋₆cycloalkyl-C₁₋₆alkyl, amino, C₁₋₆alkylamino, diC₁₋₆-alkylamino and C₃₋₆cycloalkylamino groups is optionally substituted with one to three substituents independently selected from halogen, hydroxy, and CN; and preferably R³ is hydrogen;
R⁴ is selected from C₁₋₆alkyl, C₁₋₆alkoxy-C₁₋₆alkyl, C₃₋₆cycloalkyl, C₃₋₆cycloalkyl-C₁₋₄alkyl, and C₁₋₆alkoxy, wherein each of the C₁₋₆alkyl, C₁₋₆alkoxy-C₁₋₆alkyl, C₃₋₆cycloalkyl, C₃₋₆cycloalkyl-C₁₋₄alkyl, and C₁₋₆alkoxy groups is optionally substituted with one to three substituents independently selected from halogen and CN; and wherein A³ and R⁴ taken together optionally form a ring, more preferably a 5-8-membered heterocycle, more preferably a 6-membered heterocycle; and
R⁵ is selected from C₁₋₆alkyl, C₁₋₆alkoxy, C₃₋₆cycloalkyl, C₃₋₆cycloalkyl-C₁₋₆alkyl, C₁₋₆alkoxyC₁₋₆alkyl, C₁₋₆alkylamino, diC₁₋₆alkylamino, and C₁₋₆alkylC₁₋₆alkoxyamino, wherein each of said groups is optionally substituted with one to three substituents independently selected from halogen and CN, and preferably R⁵ is selected from C₁₋₆alkyl, C₁₋₆alkoxy, C₃₋₆cycloalkyl, C₃₋₆cycloalkyl-C₁₋₆alkyl, and C₁₋₆alkoxyC₁₋₆alkyl, wherein each of said groups is optionally substituted with one to three substituents independently selected from halogen and CN.

In a preferred embodiment,

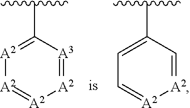

wherein R² are as defined in the present invention; preferably R² are independently selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₃₋₆cycloalkyl-C₁₋₆alkyl, C₁₋₆alkoxycarbonyl, C₁₋₆alkylaminocarbonyl, diC₁₋₆alkylaminocarbonyl, and C₁₋₆alkylcarbonyl, wherein each of the C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₃₋₆cycloalkyl-C₁₋₆alkyl, C₁₋₆ alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen, hydroxy, and CN; more preferably R² are independently selected from hydrogen, halogen, CN, C₁₋₆alkyl, C₁₋₆alkoxy, C₁₋₆alkoxy-C₁₋₆alkyl, C₁₋₆alkoxycarbonyl, and C₁₋₆alkoxy-C₁₋₆alkoxy, wherein each of the C₁₋₆alkyl, C₁₋₆ alkoxy, C₁₋₆alkoxy-C₁₋₆alkyl, C₁₋₆alkoxycarbonyl, and C₁₋₆alkoxy-C₁₋₆alkoxy groups is optionally substituted with one to three substituents independently selected from halogen, hydroxy, and CN; and even more preferably R² are independently selected from hydrogen, halogen, CN, C₁₋₆alkyl, C₁₋₆alkoxycarbonyl, and C₁₋₆alkoxy.

In a further embodiment, the compound according to the present invention is selected from:
methyl N-[5-[5-[(4-fluoro-3-methoxy-phenyl)-(methoxymethyl)carbamoyl]pyrazolo[1,5-a]pyridin-3-yl]-2-pyridyl]carbamate;
methyl N-[5-[5-[(4-chloro-3-methoxy-phenyl)-(cyanomethyl)carbamoyl]pyrazolo[1,5-a]pyridin-3-yl]-2-pyridyl]carbamate;
methyl N-[5-[5-[cyanomethyl-(4-fluoro-3-methoxy-phenyl)carbamoyl]pyrazolo[1,5-a]pyridin-3-yl]-2-pyridyl]carbamate;
methyl N-[5-[5-[(4-fluoro-3-methoxy-phenyl)-methyl-carbamoyl]pyrazolo[1,5-a]pyridin-3-yl]-2-pyridyl]carbamate;
methyl N-[4-[5-[(4-chlorophenyl)-methyl-carbamoyl]pyrazolo[1,5-a]pyridin-3-yl]phenyl]carbamate;
3-(4-acetamidophenyl)-N-(4-chlorophenyl)-N-methyl-pyrazolo[1,5-a]pyridine-5-carboxamide;
methyl N-[5-[5-[(6-methoxy-3-pyridyl)-methyl-carbamoyl]pyrazolo[1,5-a]pyridin-3-yl]-2-pyridyl]carbamate;
3-(6-acetamido-3-pyridyl)-N-(6-methoxy-3-pyridyl)-N-methyl-pyrazolo[1,5-a]pyridine-5-carboxamide;
3-(4-acetamidophenyl)-N-(6-methoxy-3-pyridyl)-N-methyl-pyrazolo[1,5-a]pyridine-5-carboxamide;
3-[4-[(2-methoxyacetyl)amino]phenyl]-N-(6-methoxy-3-pyridyl)-N-methyl-pyrazolo[1,5-a]pyridine-5-carboxamide;
3-[6-(ethylcarbamoylamino)-3-pyridyl]-N-(6-methoxy-3-pyridyl)-N-methyl-pyrazolo[1,5-a]pyridine-5-carboxamide;
methyl N-[5-[5-[(4-cyanophenyl)-methyl-carbamoyl]pyrazolo[1,5-a]pyridin-3-yl]-2-pyridyl]carbamate;
3-(6-acetamido-3-pyridyl)-N-(4-cyanophenyl)-N-methyl-pyrazolo[1,5-a]pyridine-5-carboxamide;
3-(4-acetamidophenyl)-N-(4-cyanophenyl)-N-methyl-pyrazolo[1,5-a]pyridine-5-carboxamide;
N-(4-cyanophenyl)-3-[4-[(2-methoxyacetyl)amino]phenyl]-N-methyl-pyrazolo[1,5-a]pyridine-5-carboxamide;
N-(4-cyanophenyl)-3-[6-(ethylcarbamoylamino)-3-pyridyl]-N-methyl-pyrazolo[1,5-a]pyridine-5-carboxamide;
methyl N-[5-[5-[(2-methoxy-4-pyridyl)-methyl-carbamoyl]pyrazolo[1,5-a]pyridin-3-yl]-2-pyridyl]carbamate;
3-(6-acetamido-3-pyridyl)-N-(2-methoxy-4-pyridyl)-N-methyl-pyrazolo[1,5-a]pyridine-5-carboxamide;
3-(4-acetamidophenyl)-N-(2-methoxy-4-pyridyl)-N-methyl-pyrazolo[1,5-a]pyridine-5-carboxamide;
3-[4-[(2-methoxyacetyl)amino]phenyl]-N-(2-methoxy-4-pyridyl)-N-methyl-pyrazolo[1,5-a]pyridine-5-carboxamide;
3-[6-(ethylcarbamoylamino)-3-pyridyl]-N-(2-methoxy-4-pyridyl)-N-methyl-pyrazolo[1,5-a]pyridine-5-carboxamide;
methyl N-[5-[5-[(3-fluorophenyl)-methyl-carbamoyl]pyrazolo[1,5-a]pyridin-3-yl]-2-pyridyl]carbamate;
3-(6-acetamido-3-pyridyl)-N-(3-fluorophenyl)-N-methyl-pyrazolo[1,5-a]pyridine-5-carboxamide;
3-(4-acetamidophenyl)-N-(3-fluorophenyl)-N-methyl-pyrazolo[1,5-a]pyridine-5-carboxamide;
N-(3-fluorophenyl)-3-[4-[(2-methoxyacetyl)amino]phenyl]-N-methyl-pyrazolo[1,5-a]pyridine-5-carboxamide;
3-[6-(ethylcarbamoylamino)-3-pyridyl]-N-(3-fluorophenyl)-N-methyl-pyrazolo[1,5-a]pyridine-5-carboxamide;
methyl N-[5-[5-[(4-methoxy-2-pyridyl)-methyl-carbamoyl]pyrazolo[1,5-a]pyridin-3-yl]-2-pyridyl]carbamate;
3-(6-acetamido-3-pyridyl)-N-(4-methoxy-2-pyridyl)-N-methyl-pyrazolo[1,5-a]pyridine-5-carboxamide;
3-(4-acetamidophenyl)-N-(4-methoxy-2-pyridyl)-N-methyl-pyrazolo[1,5-a]pyridine-5-carboxamide;
3-[4-[(2-methoxyacetyl)amino]phenyl]-N-(4-methoxy-2-pyridyl)-N-methyl-pyrazolo[1,5-a]pyridine-5-carboxamide;
3-[6-(ethylcarbamoylamino)-3-pyridyl]-N-(4-methoxy-2-pyridyl)-N-methyl-pyrazolo[1,5-a]pyridine-5-carboxamide;
methyl N-[5-[5-[(6-cyano-3-pyridyl)-methyl-carbamoyl]pyrazolo[1,5-a]pyridin-3-yl]-2-pyridyl]carbamate;
3-(6-acetamido-3-pyridyl)-N-(6-cyano-3-pyridyl)-N-methyl-pyrazolo[1,5-a]pyridine-5-carboxamide;
3-(4-acetamidophenyl)-N-(6-cyano-3-pyridyl)-N-methyl-pyrazolo[1,5-a]pyridine-5-carboxamide;
N-(6-cyano-3-pyridyl)-3-[4-[(2-methoxyacetyl)amino]phenyl]-N-methyl-pyrazolo[1,5-a]pyridine-5-carboxamide;
N-(6-cyano-3-pyridyl)-3-[6-(ethylcarbamoylamino)-3-pyridyl]-N-methyl-pyrazolo[1,5-a]pyridine-5-carboxamide;
methyl N-[5-[5-[(6-chloro-3-pyridyl)-methyl-carbamoyl]pyrazolo[1,5-a]pyridin-3-yl]-2-pyridyl]carbamate;
3-(4-acetamidophenyl)-N-(6-chloro-3-pyridyl)-N-methyl-pyrazolo[1,5-a]pyridine-5-carboxamide;
N-(6-chloro-3-pyridyl)-3-[4-[(2-methoxyacetyl)amino]phenyl]-N-methyl-pyrazolo[1,5-a]pyrdine-5-carboxamide;
N-(6-chloro-3-pyridyl)-3-[6-(ethylcarbamoylamino)-3-pyridyl]-N-methyl-pyrazolo[1,5-a]pyridine-5-carboxamide;
3-(6-acetamido-3-pyridyl)-N-(4-cyano-3-methoxy-phenyl)-N-methyl-pyrazolo[1,5-a]pyridine-5-carboxamide;
methyl N-[5-[5-[(4-cyano-3-methyl-phenyl)-methyl-carbamoyl]pyrazolo[1,5-a]pyridin-3-yl]-2-pyridyl]carbamate;
3-(6-acetamido-3-pyridyl)-N-(4-cyano-3-methyl-phenyl)-N-methyl-pyrazolo[1,5-a]pyridine-5-carboxamide;
3-(4-acetamidophenyl)-N-(4-cyano-3-methyl-phenyl)-N-methyl-pyrazolo[1,5-a]pyridine-5-carboxamide;
N-(4-cyano-3-methyl-phenyl)-3-[4-[(2-methoxyacetyl)amino]phenyl]-N-methyl-pyrazolo[1,5-a]pyridine-5-carboxamide;
N-(4-cyano-3-methyl-phenyl)-3-[6-(ethylcarbamoylamino)-3-pyridyl]-N-methyl-pyrazolo[1,5-a]pyridine-5-carboxamide; and
3-(6-acetamido-3-pyridyl)-N-(4-fluoro-3-methoxy-phenyl)-N-methyl-pyrazolo[1,5-a]pyridine-5-carboxamide.

The method according to the present invention has advantageous properties for protecting plants against pathogenic, such as phytopathogenic, especially fungi such as oomycetes, attack or infestation, which result in a disease and damage to the plant; particularly in instance of plants, the present invention can control, limit or prevent pathogenic damage on plant, parts of plant, plant propagation material and/or plant grown.

The compounds in Tables 1.1 to 1.45 below illustrate specific compounds of the invention.

TABLE 1.4
provides 483 compounds E1.1 to E1.483 of formula (la)

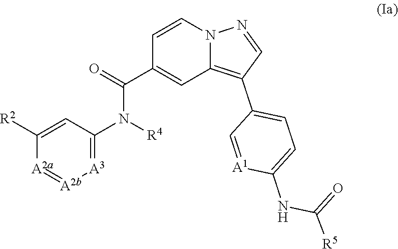

wherein A^{2a} is N, R⁵ is CH₃ and R², A³, R⁴, A¹, A^{2b} are as defined in table Z.

More particularly, table Z also includes compounds wherein A³ and R⁴ taken together form a ring. In this case, said ring is depicted in the A³ and R⁴ columns (Table Z) merged together, along the amide atom linked to the bicycle core of the formula (Ia). For example, see the A³ and R⁴ columns of the compound E1.36.

TABLE Z
Substituent definitions of R², A³, R⁴, A¹ and A^{2b}:

| Compound | A¹ | A^{2b} | A³ | R⁴ | R² |
|---|---|---|---|---|---|
| E1.1 | CH | CH | CH | CH₃ | H |
| E1.2 | CH | N | CH | CH₃ | H |
| E1.3 | N | CH | CH | CH₃ | H |
| E1.4 | N | N | CH | CH₃ | H |
| E1.5 | CH | CH | CH | CH₂CH₃ | H |
| E1.6 | CH | N | CH | CH₂CH₃ | H |
| E1.7 | N | CH | CH | CH₂CH₃ | H |
| E1.8 | N | N | CH | CH₂CH₃ | H |
| E1.9 | CH | CH | CH | CH₂OCH₃ | H |
| E1.10 | CH | N | CH | CH₂OCH₃ | H |
| E1.11 | N | CH | CH | CH₂OCH₃ | H |
| E1.12 | N | N | CH | CH₂OCH₃ | H |
| E1.13 | CH | CH | CH | CH₂CH₂OCH₃ | H |
| E1.14 | CH | N | CH | CH₂CH₂OCH₃ | H |
| E1.15 | N | CH | CH | CH₂CH₂OCH₃ | H |
| E1.16 | N | N | CH | CH₂CH₂OCH₃ | H |
| E1.17 | CH | CH | CH | CH₂CN | H |
| E1.18 | CH | N | CH | CH₂CN | H |
| E1.19 | N | CH | CH | CH₂CN | H |
| E1.20 | N | N | CH | CH₂CN | H |
| E1.21 | CH | CH | CH | CH₂CH₂CN | H |
| E1.22 | CH | N | CH | CH₂CH₂CN | H |
| E1.23 | N | CH | CH | CH₂CH₂CN | H |
| E1.24 | N | N | CH | CH₂CH₂CN | H |
| E1.25 | CH | CH | N | CH₃ | H |
| E1.26 | CH | CH | N | CH₂CH₃ | H |
| E1.27 | N | CH | N | CH₂CH₃ | H |
| E1.28 | CH | CH | N | CH₂OCH₃ | H |
| E1.29 | N | CH | N | CH₂OCH₃ | H |
| E1.30 | CH | CH | N | CH₂CH₂OCH₃ | H |
| E1.31 | N | CH | N | CH₂CH₂OCH₃ | H |
| E1.32 | CH | CH | N | CH₂CN | H |
| E1.33 | N | CH | N | CH₂CN | H |
| E1.34 | CH | CH | N | CH₂CH₂CN | H |
| E1.35 | N | CH | N | CH₂CH₂CN | H |
| E1.36 | CH | CH | 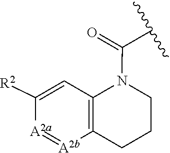 | | H |
| E1.37 | CH | N | 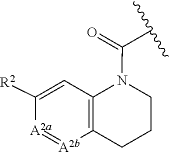 | | H |
| E1.38 | N | CH | 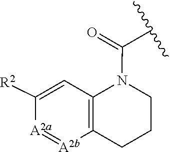 | | H |
| E1.39 | N | N | 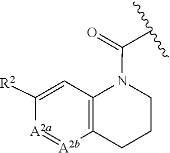 | | H |
| E1.40 | CH | CH | 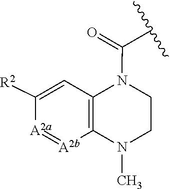 | | H |
| E1.41 | CH | N | 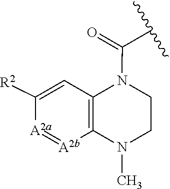 | | H |
| E1.42 | N | CH | 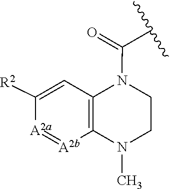 | | H |
| E1.43 | N | N | 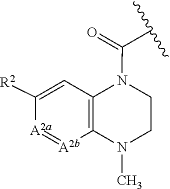 | | H |
| E1.44 | CH | CH | CH | CH₃ | CH₃ |
| E1.45 | CH | N | CH | CH₃ | CH₃ |
| E1.46 | N | CH | CH | CH₃ | CH₃ |
| E1.47 | N | N | CH | CH₃ | CH₃ |
| E1.48 | CH | CH | CH | CH₂CH₃ | CH₃ |
| E1.49 | CH | N | CH | CH₂CH₃ | CH₃ |
| E1.50 | N | CH | CH | CH₂CH₃ | CH₃ |
| E1.51 | N | N | CH | CH₂CH₃ | CH₃ |
| E1.52 | CH | CH | CH | CH₂OCH₃ | CH₃ |
| E1.53 | CH | N | CH | CH₂OCH₃ | CH₃ |
| E1.54 | N | CH | CH | CH₂OCH₃ | CH₃ |
| E1.55 | N | N | CH | CH₂OCH₃ | CH₃ |
| E1.56 | CH | CH | CH | CH₂CH₂OCH₃ | CH₃ |
| E1.57 | CH | N | CH | CH₂CH₂OCH₃ | CH₃ |
| E1.58 | N | CH | CH | CH₂CH₂OCH₃ | CH₃ |
| E1.59 | N | N | CH | CH₂CH₂OCH₃ | CH₃ |
| E1.60 | CH | CH | CH | CH₂CN | CH₃ |
| E1.61 | CH | N | CH | CH₂CN | CH₃ |
| E1.62 | N | CH | CH | CH₂CN | CH₃ |
| E1.63 | N | N | CH | CH₂CN | CH₃ |
| E1.64 | CH | CH | CH | CH₂CH₂CN | CH₃ |
| E1.65 | CH | N | CH | CH₂CH₂CN | CH₃ |
| E1.66 | N | CH | CH | CH₂CH₂CN | CH₃ |
| E1.67 | N | N | CH | CH₂CH₂CN | CH₃ |
| E1.68 | CH | CH | N | CH₃ | CH₃ |
| E1.69 | N | CH | N | CH₃ | CH₃ |
| E1.70 | CH | CH | N | CH₂CH₃ | CH₃ |
| E1.71 | N | CH | N | CH₂CH₃ | CH₃ |
| E1.72 | CH | CH | N | CH₂OCH₃ | CH₃ |
| E1.73 | N | CH | N | CH₂OCH₃ | CH₃ |
| E1.74 | CH | CH | N | CH₂CH₂OCH₃ | CH₃ |
| E1.75 | N | CH | N | CH₂CH₂OCH₃ | CH₃ |
| E1.76 | CH | CH | N | CH₂CN | CH₃ |
| E1.77 | N | CH | N | CH₂CN | CH₃ |
| E1.78 | CH | CH | N | CH₂CH₂CN | CH₃ |
| E1.79 | N | CH | N | CH₂CH₂CN | CH₃ |
| E1.80 | CH | CH | 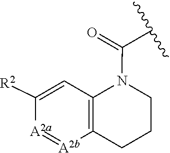 | | CH₃ |
| E1.81 | CH | N | 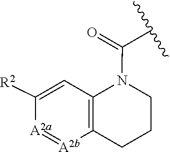 | | CH₃ |
| E1.82 | N | CH | 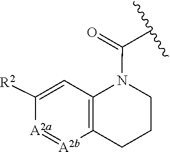 | | CH₃ |
| E1.83 | N | N | 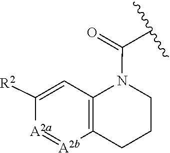 | | CH₃ |
| E1.84 | CH | CH | 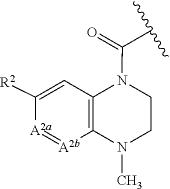 | | CH₃ |
| E1.85 | CH | N | 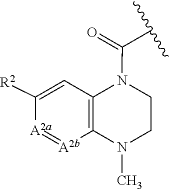 | | CH₃ |
| E1.86 | N | CH | 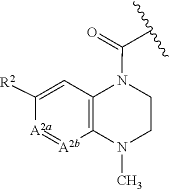 | | CH₃ |
| E1.87 | N | N | 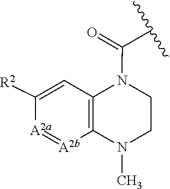 | | CH₃ |
| E1.88 | CH | CH | CH | CH₃ | CH₂CH₃ |
| E1.89 | CH | N | CH | CH₃ | CH₂CH₃ |
| E1.90 | N | CH | CH | CH₃ | CH₂CH₃ |
| E1.91 | N | N | CH | CH₃ | CH₂CH₃ |
| E1.92 | CH | CH | CH | CH₂CH₃ | CH₂CH₃ |
| E1.93 | CH | N | CH | CH₂CH₃ | CH₂CH₃ |
| E1.94 | N | CH | CH | CH₂CH₃ | CH₂CH₃ |
| E1.95 | N | N | CH | CH₂CH₃ | CH₂CH₃ |
| E1.96 | CH | CH | CH | CH₂OCH₃ | CH₂CH₃ |
| E1.97 | CH | N | CH | CH₂OCH₃ | CH₂CH₃ |
| E1.98 | N | CH | CH | CH₂OCH₃ | CH₂CH₃ |
| E1.99 | N | N | CH | CH₂OCH₃ | CH₂CH₃ |
| E1.100 | CH | CH | CH | CH₂CH₂OCH₃ | CH₂CH₃ |
| E1.101 | CH | N | CH | CH₂CH₂OCH₃ | CH₂CH₃ |
| E1.102 | N | CH | CH | CH₂CH₂OCH₃ | CH₂CH₃ |
| E1.103 | N | N | CH | CH₂CH₂OCH₃ | CH₂CH₃ |
| E1.104 | CH | CH | CH | CH₂CN | CH₂CH₃ |
| E1.105 | CH | N | CH | CH₂CN | CH₂CH₃ |
| E1.106 | N | CH | CH | CH₂CN | CH₂CH₃ |
| E1.107 | N | N | CH | CH₂CN | CH₂CH₃ |
| E1.108 | CH | CH | CH | CH₂CH₂CN | CH₂CH₃ |
| E1.109 | CH | N | CH | CH₂CH₂CN | CH₂CH₃ |
| E1.110 | N | CH | CH | CH₂CH₂CN | CH₂CH₃ |
| E1.111 | N | N | CH | CH₂CH₂CN | CH₂CH₃ |
| E1.112 | CH | CH | N | CH₃ | CH₂CH₃ |
| E1.113 | N | CH | N | CH₃ | CH₂CH₃ |
| E1.114 | CH | CH | N | CH₂CH₃ | CH₂CH₃ |
| E1.115 | N | CH | N | CH₂CH₃ | CH₂CH₃ |
| E1.116 | CH | CH | N | CH₂OCH₃ | CH₂CH₃ |
| E1.117 | N | CH | N | CH₂OCH₃ | CH₂CH₃ |
| E1.118 | CH | CH | N | CH₂CH₂OCH₃ | CH₂CH₃ |
| E1.119 | N | CH | N | CH₂CH₂OCH₃ | CH₂CH₃ |
| E1.120 | CH | CH | N | CH₂CN | CH₂CH₃ |
| E1.121 | N | CH | N | CH₂CN | CH₂CH₃ |
| E1.122 | CH | CH | N | CH₂CH₂CN | CH₂CH₃ |
| E1.123 | N | CH | N | CH₂CH₂CN | CH₂CH₃ |
| E1.124 | CH | CH | 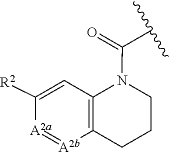 | | CH₂CH₃ |
| E1.125 | CH | N | 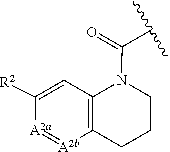 | | CH₂CH₃ |
| E1.126 | N | CH | 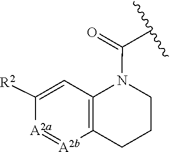 | | CH₂CH₃ |
| E1.127 | N | N | 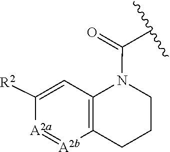 | | CH₂CH₃ |
| E1.128 | CH | CH | 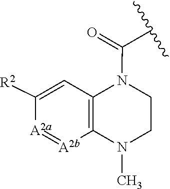 | | CH₂CH₃ |
| E1.129 | CH | N | 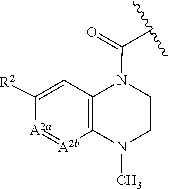 | | CH₂CH₃ |
| E1.130 | N | CH | 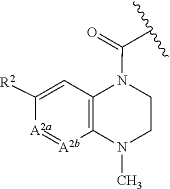 | | CH₂CH₃ |
| E1.131 | N | N | 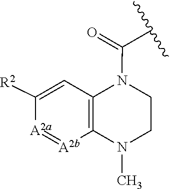 | | CH₂CH₃ |
| E1.132 | CH | CH | CH | CH₃ | F |
| E1.133 | CH | N | CH | CH₃ | F |
| E1.134 | N | CH | CH | CH₃ | F |
| E1.135 | N | N | CH | CH₃ | F |
| E1.136 | CH | CH | CH | CH₂CH₃ | F |
| E1.137 | CH | N | CH | CH₂CH₃ | F |
| E1.138 | N | CH | CH | CH₂CH₃ | F |
| E1.139 | N | N | CH | CH₂CH₃ | F |
| E1.140 | CH | CH | CH | CH₂OCH₃ | F |
| E1.141 | CH | N | CH | CH₂OCH₃ | F |
| E1.142 | N | CH | CH | CH₂OCH₃ | F |
| E1.143 | N | N | CH | CH₂OCH₃ | F |
| E1.144 | CH | CH | CH | CH₂CH₂OCH₃ | F |
| E1.145 | CH | N | CH | CH₂CH₂OCH₃ | F |
| E1.146 | N | CH | CH | CH₂CH₂OCH₃ | F |
| E1.147 | N | N | CH | CH₂CH₂OCH₃ | F |
| E1.148 | CH | CH | CH | CH₂CN | F |
| E1.149 | CH | N | CH | CH₂CN | F |
| E1.150 | N | CH | CH | CH₂CN | F |
| E1.151 | N | N | CH | CH₂CN | F |
| E1.152 | CH | CH | CH | CH₂CH₂CN | F |
| E1.153 | CH | N | CH | CH₂CH₂CN | F |
| E1.154 | N | CH | CH | CH₂CH₂CN | F |
| E1.155 | N | N | CH | CH₂CH₂CN | F |
| E1.156 | CH | CH | N | CH₃ | F |
| E1.157 | N | CH | N | CH₃ | F |
| E1.158 | CH | CH | N | CH₂CH₃ | F |
| E1.159 | N | CH | N | CH₂CH₃ | F |
| E1.160 | CH | CH | N | CH₂OCH₃ | F |
| E1.161 | N | CH | N | CH₂OCH₃ | F |
| E1.162 | CH | CH | N | CH₂CH₂OCH₃ | F |
| E1.163 | N | CH | N | CH₂CH₂OCH₃ | F |
| E1.164 | CH | CH | N | CH₂CN | F |
| E1.165 | N | CH | N | CH₂CN | F |
| E1.166 | CH | CH | N | CH₂CH₂CN | F |
| E1.167 | N | CH | N | CH₂CH₂CN | F |
| E1.168 | CH | CH | 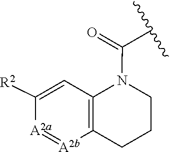 | | F |
| E1.169 | CH | N | 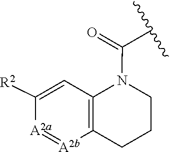 | | F |
| E1.170 | N | CH | 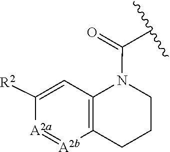 | | F |
| E1.171 | N | N | 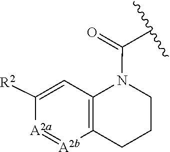 | | F |
| E1.172 | CH | CH | 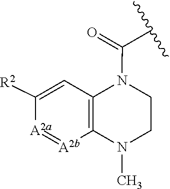 | | F |
| E1.173 | CH | N | 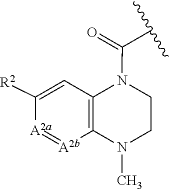 | | F |
| E1.174 | N | CH | 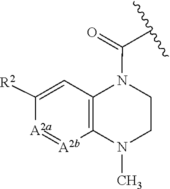 | | F |
| E1.175 | N | N | 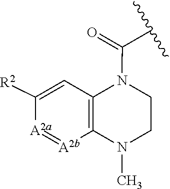 | | F |
| E1.176 | CH | CH | CH | CH₃ | Cl |
| E1.177 | CH | N | CH | CH₃ | Cl |
| E1.178 | N | CH | CH | CH₃ | Cl |
| E1.179 | N | N | CH | CH₃ | Cl |
| E1.180 | CH | CH | CH | CH₂CH₃ | Cl |
| E1.181 | CH | N | CH | CH₂CH₃ | Cl |
| E1.182 | N | CH | CH | CH₂CH₃ | Cl |
| E1.183 | N | N | CH | CH₂CH₃ | Cl |
| E1.184 | CH | CH | CH | CH₂OCH₃ | Cl |
| E1.185 | CH | N | CH | CH₂OCH₃ | Cl |
| E1.186 | N | CH | CH | CH₂OCH₃ | Cl |
| E1.187 | N | N | CH | CH₂OCH₃ | Cl |
| E1.188 | CH | CH | CH | CH₂CH₂OCH₃ | Cl |
| E1.189 | CH | N | CH | CH₂CH₂OCH₃ | Cl |
| E1.190 | N | CH | CH | CH₂CH₂OCH₃ | Cl |
| E1.191 | N | N | CH | CH₂CH₂OCH₃ | Cl |
| E1.192 | CH | CH | CH | CH₂CN | Cl |
| E1.193 | CH | N | CH | CH₂CN | Cl |
| E1.194 | N | CH | CH | CH₂CN | Cl |
| E1.195 | N | N | CH | CH₂CN | Cl |
| E1.196 | CH | CH | CH | CH₂CH₂CN | Cl |
| E1.197 | CH | N | CH | CH₂CH₂CN | Cl |
| E1.198 | N | CH | CH | CH₂CH₂CN | Cl |
| E1.199 | N | N | CH | CH₂CH₂CN | Cl |
| E1.200 | CH | CH | N | CH₃ | Cl |
| E1.201 | N | CH | N | CH₃ | Cl |
| E1.202 | CH | CH | N | CH₂CH₃ | Cl |
| E1.203 | N | CH | N | CH₂CH₃ | Cl |
| E1.204 | CH | CH | N | CH₂OCH₃ | Cl |
| E1.205 | N | CH | N | CH₂OCH₃ | Cl |
| E1.206 | CH | CH | N | CH₂CH₂OCH₃ | Cl |
| E1.207 | N | CH | N | CH₂CH₂OCH₃ | Cl |
| E1.208 | CH | CH | N | CH₂CN | Cl |
| E1.209 | N | CH | N | CH₂CN | Cl |
| E1.210 | CH | CH | N | CH₂CH₂CN | Cl |
| E1.211 | N | CH | N | CH₂CH₂CN | Cl |
| E1.212 | CH | CH | 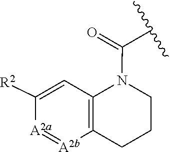 | | Cl |
| E1.213 | CH | N | 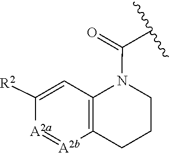 | | Cl |
| E1.214 | N | CH | 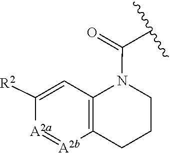 | | Cl |
| E1.215 | N | N | 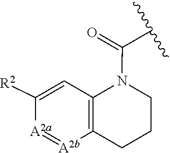 | | Cl |
| E1.216 | CH | CH | 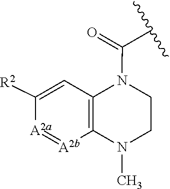 | | Cl |
| E1.217 | CH | N | 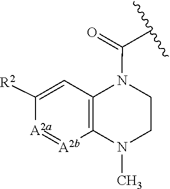 | | Cl |
| E1.218 | N | CH | 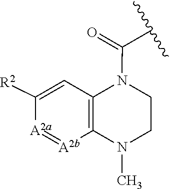 | | Cl |
| E1.219 | N | N | 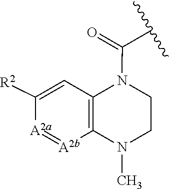 | | Cl |
| E1.220 | CH | CH | CH | CH₃ | Br |
| E1.221 | CH | N | CH | CH₃ | Br |
| E1.222 | N | CH | CH | CH₃ | Br |
| E1.223 | N | N | CH | CH₃ | Br |
| E1.224 | CH | CH | CH | CH₂CH₃ | Br |
| E1.225 | CH | N | CH | CH₂CH₃ | Br |
| E1.226 | N | CH | CH | CH₂CH₃ | Br |
| E1.227 | N | N | CH | CH₂CH₃ | Br |
| E1.228 | CH | CH | CH | CH₂OCH₃ | Br |
| E1.229 | CH | N | CH | CH₂OCH₃ | Br |
| E1.230 | N | CH | CH | CH₂OCH₃ | Br |
| E1.231 | N | N | CH | CH₂OCH₃ | Br |
| E1.232 | CH | CH | CH | CH₂CH₂OCH₃ | Br |
| E1.233 | CH | N | CH | CH₂CH₂OCH₃ | Br |
| E1.234 | N | CH | CH | CH₂CH₂OCH₃ | Br |
| E1.235 | N | N | CH | CH₂CH₂OCH₃ | Br |
| E1.236 | CH | CH | CH | CH₂CN | Br |
| E1.237 | CH | N | CH | CH₂CN | Br |
| E1.238 | N | CH | CH | CH₂CN | Br |
| E1.239 | N | N | CH | CH₂CN | Br |
| E1.240 | CH | CH | CH | CH₂CH₂CN | Br |
| E1.241 | CH | N | CH | CH₂CH₂CN | Br |
| E1.242 | N | CH | CH | CH₂CH₂CN | Br |
| E1.243 | N | N | CH | CH₂CH₂CN | Br |
| E1.244 | CH | CH | N | CH₃ | Br |
| E1.245 | N | CH | N | CH₃ | Br |
| E1.246 | CH | CH | N | CH₂CH₃ | Br |
| E1.247 | N | CH | N | CH₂CH₃ | Br |
| E1.248 | CH | CH | N | CH₂OCH₃ | Br |
| E1.249 | N | CH | N | CH₂OCH₃ | Br |
| E1.250 | CH | CH | N | CH₂CH₂OCH₃ | Br |
| E1.251 | N | CH | N | CH₂CH₂OCH₃ | Br |
| E1.252 | CH | CH | N | CH₂CN | Br |
| E1.253 | N | CH | N | CH₂CN | Br |
| E1.254 | CH | CH | N | CH₂CH₂CN | Br |
| E1.255 | N | CH | N | CH₂CH₂CN | Br |
| E1.256 | CH | CH | 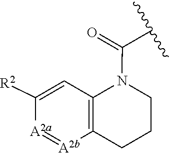 | | Br |
| E1.257 | CH | N | 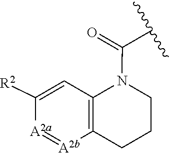 | | Br |
| E1.258 | N | CH | 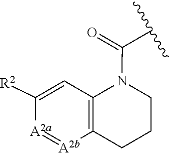 | | Br |
| E1.259 | N | N | 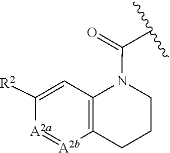 | | Br |
| E1.260 | CH | CH | 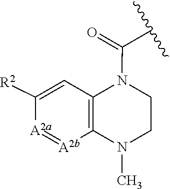 | | Br |
| E1.261 | CH | N | 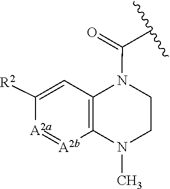 | | Br |
| E1.262 | N | CH | 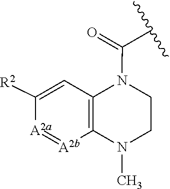 | | Br |
| E1.263 | N | N | 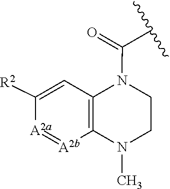 | | Br |
| E1.264 | CH | CH | CH | CH₃ | CN |
| E1.265 | CH | N | CH | CH₃ | CN |
| E1.266 | N | CH | CH | CH₃ | CN |
| E1.267 | N | N | CH | CH₃ | CN |
| E1.268 | CH | CH | CH | CH₂CH₃ | CN |
| E1.269 | CH | N | CH | CH₂CH₃ | CN |
| E1.270 | N | CH | CH | CH₂CH₃ | CN |
| E1.271 | N | N | CH | CH₂CH₃ | CN |
| E1.272 | CH | CH | CH | CH₂OCH₃ | CN |
| E1.273 | CH | N | CH | CH₂OCH₃ | CN |
| E1.274 | N | CH | CH | CH₂OCH₃ | CN |
| E1.275 | N | N | CH | CH₂OCH₃ | CN |
| E1.276 | CH | CH | CH | CH₂CH₂OCH₃ | CN |
| E1.277 | CH | N | CH | CH₂CH₂OCH₃ | CN |
| E1.278 | N | CH | CH | CH₂CH₂OCH₃ | CN |
| E1.279 | N | N | CH | CH₂CH₂OCH₃ | CN |
| E1.280 | CH | CH | CH | CH₂CN | CN |
| E1.281 | CH | N | CH | CH₂CN | CN |
| E1.282 | N | CH | CH | CH₂CN | CN |
| E1.283 | N | N | CH | CH₂CN | CN |
| E1.284 | CH | CH | CH | CH₂CH₂CN | CN |
| E1.285 | CH | N | CH | CH₂CH₂CN | CN |
| E1.286 | N | CH | CH | CH₂CH₂CN | CN |
| E1.287 | N | N | CH | CH₂CH₂CN | CN |
| E1.288 | CH | CH | N | CH₃ | CN |
| E1.289 | N | CH | N | CH₃ | CN |
| E1.290 | CH | CH | N | CH₂CH₃ | CN |
| E1.291 | N | CH | N | CH₂CH₃ | CN |
| E1.292 | CH | CH | N | CH₂OCH₃ | CN |
| E1.293 | N | CH | N | CH₂OCH₃ | CN |
| E1.294 | CH | CH | N | CH₂CH₂OCH₃ | CN |
| E1.295 | N | CH | N | CH₂CH₂OCH₃ | CN |
| E1.296 | CH | CH | N | CH₂CN | CN |
| E1.297 | N | CH | N | CH₂CN | CN |
| E1.298 | CH | CH | N | CH₂CH₂CN | CN |
| E1.299 | N | CH | N | CH₂CH₂CN | CN |
| E1.300 | CH | CH | 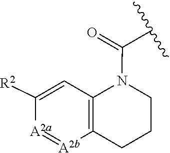 | | CN |
| E1.301 | CH | N | 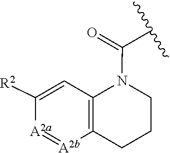 | | CN |
| E1.302 | N | CH | 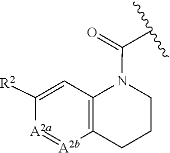 | | CN |
| E1.303 | N | N | 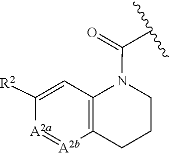 | | CN |
| E1.304 | CH | CH | 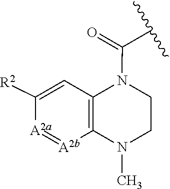 | | CN |
| E1.305 | CH | N | 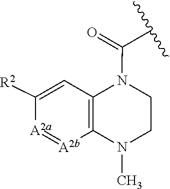 | | CN |
| E1.306 | N | CH | 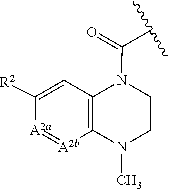 | | CN |
| E1.307 | N | N | 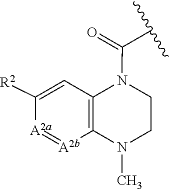 | | CN |
| E1.308 | CH | CH | CH | CH₃ | OCH₃ |
| E1.309 | CH | N | CH | CH₃ | OCH₃ |
| E1.310 | N | CH | CH | CH₃ | OCH₃ |
| E1.311 | N | N | CH | CH₃ | OCH₃ |
| E1.312 | CH | CH | CH | CH₂CH₃ | OCH₃ |
| E1.313 | CH | N | CH | CH₂CH₃ | OCH₃ |
| E1.314 | N | CH | CH | CH₂CH₃ | OCH₃ |
| E1.315 | N | N | CH | CH₂CH₃ | OCH₃ |
| E1.316 | CH | CH | CH | CH₂OCH₃ | OCH₃ |
| E1.317 | CH | N | CH | CH₂OCH₃ | OCH₃ |
| E1.318 | N | CH | CH | CH₂OCH₃ | OCH₃ |
| E1.319 | N | N | CH | CH₂OCH₃ | OCH₃ |
| E1.320 | CH | CH | CH | CH₂CH₂OCH₃ | OCH₃ |
| E1.321 | CH | N | CH | CH₂CH₂OCH₃ | OCH₃ |
| E1.322 | N | CH | CH | CH₂CH₂OCH₃ | OCH₃ |
| E1.323 | N | N | CH | CH₂CH₂OCH₃ | OCH₃ |
| E1.324 | CH | CH | CH | CH₂CN | OCH₃ |
| E1.325 | CH | N | CH | CH₂CN | OCH₃ |
| E1.326 | N | CH | CH | CH₂CN | OCH₃ |
| E1.327 | N | N | CH | CH₂CN | OCH₃ |
| E1.328 | CH | CH | CH | CH₂CH₂CN | OCH₃ |
| E1.329 | CH | N | CH | CH₂CH₂CN | OCH₃ |
| E1.330 | N | CH | CH | CH₂CH₂CN | OCH₃ |
| E1.331 | N | N | CH | CH₂CH₂CN | OCH₃ |
| E1.332 | CH | CH | N | CH₃ | OCH₃ |
| E1.333 | N | CH | N | CH₃ | OCH₃ |
| E1.334 | CH | CH | N | CH₂CH₃ | OCH₃ |
| E1.335 | N | CH | N | CH₂CH₃ | OCH₃ |
| E1.336 | CH | CH | N | CH₂OCH₃ | OCH₃ |
| E1.337 | N | CH | N | CH₂OCH₃ | OCH₃ |
| E1.338 | CH | CH | N | CH₂CH₂OCH₃ | OCH₃ |
| E1.339 | N | CH | N | CH₂CH₂OCH₃ | OCH₃ |
| E1.340 | CH | CH | N | CH₂CN | OCH₃ |
| E1.341 | N | CH | N | CH₂CN | OCH₃ |
| E1.342 | CH | CH | N | CH₂CH₂CN | OCH₃ |
| E1.343 | N | CH | N | CH₂CH₂CN | OCH₃ |
| E1.344 | CH | CH | 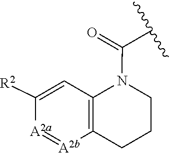 | | OCH₃ |
| E1.345 | CH | N | 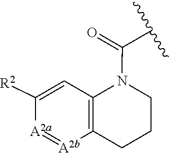 | | OCH₃ |
| E1.346 | N | CH | 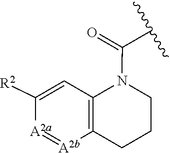 | | OCH₃ |
| E1.347 | N | N | 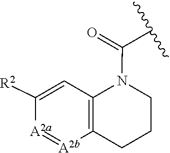 | | OCH₃ |
| E1.348 | CH | CH | 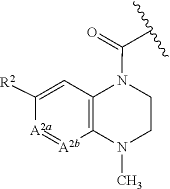 | | OCH₃ |
| E1.349 | CH | N | 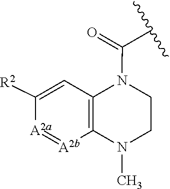 | | OCH₃ |
| E1.350 | N | CH | 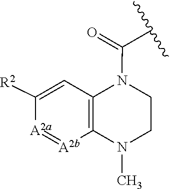 | | OCH₃ |
| E1.351 | N | N | 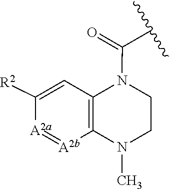 | | OCH₃ |
| E1.352 | CH | CH | CH | CH₃ | OCH₂CH₃ |
| E1.353 | CH | N | CH | CH₃ | OCH₂CH₃ |
| E1.354 | N | CH | CH | CH₃ | OCH₂CH₃ |
| E1.355 | N | N | CH | CH₃ | OCH₂CH₃ |
| E1.356 | CH | CH | CH | CH₂CH₃ | OCH₂CH₃ |
| E1.357 | CH | N | CH | CH₂CH₃ | OCH₂CH₃ |
| E1.358 | N | CH | CH | CH₂CH₃ | OCH₂CH₃ |
| E1.359 | N | N | CH | CH₂CH₃ | OCH₂CH₃ |
| E1.360 | CH | CH | CH | CH₂OCH₃ | OCH₂CH₃ |
| E1.361 | CH | N | CH | CH₂OCH₃ | OCH₂CH₃ |
| E1.362 | N | CH | CH | CH₂OCH₃ | OCH₂CH₃ |
| E1.363 | N | N | CH | CH₂OCH₃ | OCH₂CH₃ |
| E1.364 | CH | CH | CH | CH₂CH₂OCH₃ | OCH₂CH₃ |
| E1.365 | CH | N | CH | CH₂CH₂OCH₃ | OCH₂CH₃ |
| E1.366 | N | CH | CH | CH₂CH₂OCH₃ | OCH₂CH₃ |
| E1.367 | N | N | CH | CH₂CH₂OCH₃ | OCH₂CH₃ |
| E1.368 | CH | CH | CH | CH₂CN | OCH₂CH₃ |
| E1.369 | CH | N | CH | CH₂CN | OCH₂CH₃ |
| E1.370 | N | CH | CH | CH₂CN | OCH₂CH₃ |
| E1.371 | N | N | CH | CH₂CN | OCH₂CH₃ |
| E1.372 | CH | CH | CH | CH₂CH₂CN | OCH₂CH₃ |
| E1.373 | CH | N | CH | CH₂CH₂CN | OCH₂CH₃ |
| E1.374 | N | CH | CH | CH₂CH₂CN | OCH₂CH₃ |
| E1.375 | N | N | CH | CH₂CH₂CN | OCH₂CH₃ |
| E1.376 | CH | CH | N | CH₃ | OCH₂CH₃ |
| E1.377 | N | CH | N | CH₃ | OCH₂CH₃ |
| E1.378 | CH | CH | N | CH₂CH₃ | OCH₂CH₃ |
| E1.379 | N | CH | N | CH₂CH₃ | OCH₂CH₃ |
| E1.380 | CH | CH | N | CH₂OCH₃ | OCH₂CH₃ |
| E1.381 | N | CH | N | CH₂OCH₃ | OCH₂CH₃ |
| E1.382 | CH | CH | N | CH₂CH₂OCH₃ | OCH₂CH₃ |
| E1.383 | N | CH | N | CH₂CH₂OCH₃ | OCH₂CH₃ |
| E1.384 | CH | CH | N | CH₂CN | OCH₂CH₃ |
| E1.385 | N | CH | N | CH₂CN | OCH₂CH₃ |
| E1.386 | CH | CH | N | CH₂CH₂CN | OCH₂CH₃ |
| E1.387 | N | CH | N | CH₂CH₂CN | OCH₂CH₃ |
| E1.388 | CH | CH | 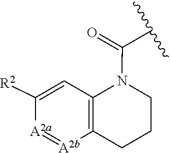 | | OCH₂CH₃ |
| E1.389 | CH | N | 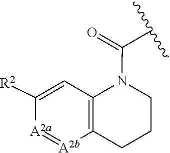 | | OCH₂CH₃ |
| E1.390 | N | CH | 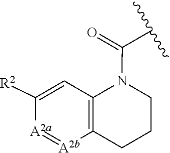 | | OCH₂CH₃ |
| E1.391 | N | N | 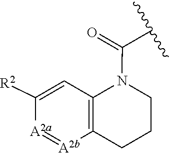 | | OCH₂CH₃ |
| E1.392 | CH | CH | 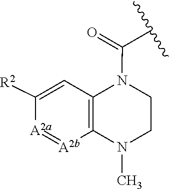 | | OCH₂CH₃ |
| E1.393 | CH | N | 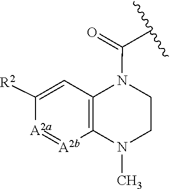 | | OCH₂CH₃ |
| E1.394 | N | CH | 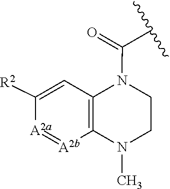 | | OCH₂CH₃ |
| E1.395 | N | N | 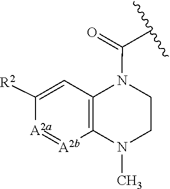 | | OCH₂CH₃ |
| E1.396 | CH | CH | CH | CH₃ | OCH₂CH₂OCH₃ |
| E1.397 | CH | N | CH | CH₃ | OCH₂CH₂OCH₃ |
| E1.398 | N | CH | CH | CH₃ | OCH₂CH₂OCH₃ |
| E1.399 | N | N | CH | CH₃ | OCH₂CH₂OCH₃ |
| E1.400 | CH | CH | CH | CH₂CH₃ | OCH₂CH₂OCH₃ |
| E1.401 | CH | N | CH | CH₂CH₃ | OCH₂CH₂OCH₃ |
| E1.402 | N | CH | CH | CH₂CH₃ | OCH₂CH₂OCH₃ |
| E1.403 | N | N | CH | CH₂CH₃ | OCH₂CH₂OCH₃ |
| E1.404 | CH | CH | CH | CH₂OCH₃ | OCH₂CH₂OCH₃ |
| E1.405 | CH | N | CH | CH₂OCH₃ | OCH₂CH₂OCH₃ |
| E1.406 | N | CH | CH | CH₂OCH₃ | OCH₂CH₂OCH₃ |
| E1.407 | N | N | CH | CH₂OCH₃ | OCH₂CH₂OCH₃ |
| E1.408 | CH | CH | CH | CH₂CH₂OCH₃ | OCH₂CH₂OCH₃ |
| E1.409 | CH | N | CH | CH₂CH₂OCH₃ | OCH₂CH₂OCH₃ |
| E1.410 | N | CH | CH | CH₂CH₂OCH₃ | OCH₂CH₂OCH₃ |
| E1.411 | N | N | CH | CH₂CH₂OCH₃ | OCH₂CH₂OCH₃ |
| E1.412 | CH | CH | CH | CH₂CN | OCH₂CH₂OCH₃ |
| E1.413 | CH | N | CH | CH₂CN | OCH₂CH₂OCH₃ |
| E1.414 | N | CH | CH | CH₂CN | OCH₂CH₂OCH₃ |
| E1.415 | N | N | CH | CH₂CN | OCH₂CH₂OCH₃ |
| E1.416 | CH | CH | CH | CH₂CH₂CN | OCH₂CH₂OCH₃ |
| E1.417 | CH | N | CH | CH₂CH₂CN | OCH₂CH₂OCH₃ |
| E1.418 | N | CH | CH | CH₂CH₂CN | OCH₂CH₂OCH₃ |
| E1.419 | N | N | CH | CH₂CH₂CN | OCH₂CH₂OCH₃ |
| E1.420 | CH | CH | N | CH₃ | OCH₂CH₂OCH₃ |
| E1.421 | N | CH | N | CH₃ | OCH₂CH₂OCH₃ |
| E1.422 | CH | CH | N | CH₂CH₃ | OCH₂CH₂OCH₃ |
| E1.423 | N | CH | N | CH₂CH₃ | OCH₂CH₂OCH₃ |
| E1.424 | CH | CH | N | CH₂OCH₃ | OCH₂CH₂OCH₃ |
| E1.425 | N | CH | N | CH₂OCH₃ | OCH₂CH₂OCH₃ |
| E1.426 | CH | CH | N | CH₂CH₂OCH₃ | OCH₂CH₂OCH₃ |
| E1.427 | N | CH | N | CH₂CH₂OCH₃ | OCH₂CH₂OCH₃ |
| E1.428 | CH | CH | N | CH₂CN | OCH₂CH₂OCH₃ |
| E1.429 | N | CH | N | CH₂CN | OCH₂CH₂OCH₃ |
| E1.430 | CH | CH | N | CH₂CH₂CN | OCH₂CH₂OCH₃ |
| E1.431 | N | CH | N | CH₂CH₂CN | OCH₂CH₂OCH₃ |
| E1.432 | CH | CH | 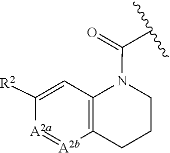 | | OCH₂CH₂OCH₃ |
| E1.433 | CH | N | 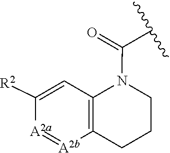 | | OCH₂CH₂OCH₃ |
| E1.434 | N | CH | 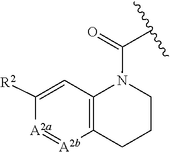 | | OCH₂CH₂OCH₃ |
| E1.435 | N | N | 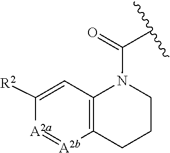 | | OCH₂CH₂OCH₃ |
| E1.436 | CH | CH | 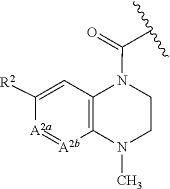 | | OCH₂CH₂OCH₃ |
| E1.437 | CH | N | 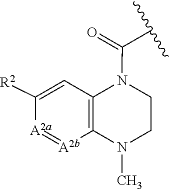 | | OCH₂CH₂OCH₃ |
| E1.438 | N | CH | 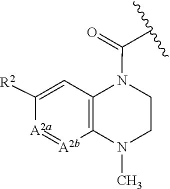 | | OCH₂CH₂OCH₃ |
| E1.439 | N | N | 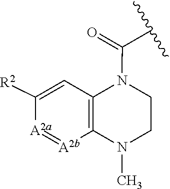 | | OCH₂CH₂OCH₃ |
| E1.440 | CH | CH | CH | CH₃ | CO₂CH₃ |
| E1.441 | CH | N | CH | CH₃ | CO₂CH₃ |
| E1.442 | N | CH | CH | CH₃ | CO₂CH₃ |
| E1.443 | N | N | CH | CH₃ | CO₂CH₃ |
| E1.444 | CH | CH | CH | CH₂CH₃ | CO₂CH₃ |
| E1.445 | CH | N | CH | CH₂CH₃ | CO₂CH₃ |
| E1.446 | N | CH | CH | CH₂CH₃ | CO₂CH₃ |
| E1.447 | N | N | CH | CH₂CH₃ | CO₂CH₃ |
| E1.448 | CH | CH | CH | CH₂OCH₃ | CO₂CH₃ |
| E1.449 | CH | N | CH | CH₂OCH₃ | CO₂CH₃ |
| E1.450 | N | CH | CH | CH₂OCH₃ | CO₂CH₃ |
| E1.451 | N | N | CH | CH₂OCH₃ | CO₂CH₃ |
| E1.452 | CH | CH | CH | CH₂CH₂OCH₃ | CO₂CH₃ |
| E1.453 | CH | N | CH | CH₂CH₂OCH₃ | CO₂CH₃ |
| E1.454 | N | CH | CH | CH₂CH₂OCH₃ | CO₂CH₃ |
| E1.455 | N | N | CH | CH₂CH₂OCH₃ | CO₂CH₃ |
| E1.456 | CH | CH | CH | CH₂CN | CO₂CH₃ |
| E1.457 | CH | N | CH | CH₂CN | CO₂CH₃ |
| E1.458 | N | CH | CH | CH₂CN | CO₂CH₃ |
| E1.459 | N | N | CH | CH₂CN | CO₂CH₃ |
| E1.460 | CH | CH | CH | CH₂CH₂CN | CO₂CH₃ |
| E1.461 | CH | N | CH | CH₂CH₂CN | CO₂CH₃ |
| E1.462 | N | CH | CH | CH₂CH₂CN | CO₂CH₃ |
| E1.463 | N | N | CH | CH₂CH₂CN | CO₂CH₃ |
| E1.464 | CH | CH | N | CH₃ | CO₂CH₃ |
| E1.465 | N | CH | N | CH₃ | CO₂CH₃ |
| E1.466 | CH | CH | N | CH₂CH₃ | CO₂CH₃ |
| E1.467 | N | CH | N | CH₂CH₃ | CO₂CH₃ |
| E1.468 | CH | CH | N | CH₂OCH₃ | CO₂CH₃ |
| E1.469 | N | CH | N | CH₂OCH₃ | CO₂CH₃ |
| E1.470 | CH | CH | N | CH₂CH₂OCH₃ | CO₂CH₃ |
| E1.471 | N | CH | N | CH₂CH₂OCH₃ | CO₂CH₃ |
| E1.472 | CH | CH | N | CH₂CN | CO₂CH₃ |
| E1.473 | N | CH | N | CH₂CN | CO₂CH₃ |
| E1.474 | CH | CH | N | CH₂CH₂CN | CO₂CH₃ |
| E1.475 | N | CH | N | CH₂CH₂CN | CO₂CH₃ |

| | | | | |
|---|---|---|---|---|
| E1.476 | CH | CH | 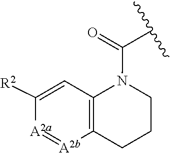 | CO₂CH₃ |
| E1.477 | CH | N | 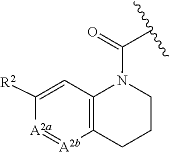 | CO₂CH₃ |
| E1.478 | N | CH | 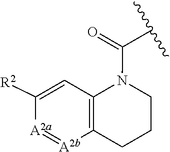 | CO₂CH₃ |
| E1.479 | N | N | 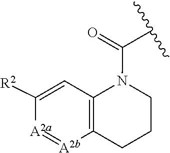 | CO₂CH₃ |
| E1.480 | CH | CH | 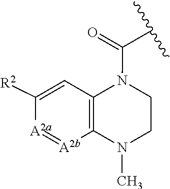 | CO₂CH₃ |
| E1.481 | CH | N | 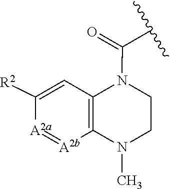 | CO₂CH₃ |
| E1.482 | N | CH | 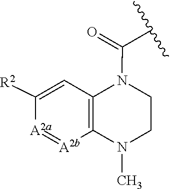 | CO₂CH₃ |
| E1.483 | N | N | 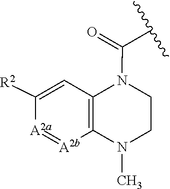 | CO₂CH₃ |

Table 1.2 provides 483 compounds E2.1 to E2.483 of formula (Ia) wherein A^{2a} is N, R⁵ is OCH₃ and R², A³, R⁴, A¹, A^{2b} are as defined in table Z.

Table 1.3 provides 483 compounds E3.1 to E3.483 of formula (Ia) wherein A^{2a} is N, R⁵ is cyclopropyl and R², A³, R⁴, A¹, A^{2b} are as defined in table Z.

Table 1.4 provides 483 compounds E4.1 to E4.483 of formula (Ia) wherein A^{2a} is N, R⁵ is CH₂OCH₃ and R², A³, R⁴, A¹, A^{2b} are as defined in table Z.

Table 1.5 provides 483 compounds E5.1 to E5.483 of formula (Ia) wherein A^{2a} is N, R⁵ is NHCH₂CH₃ and R², A³, R⁴, A¹, A^{2b} are as defined in table Z.

Table 1.6 provides 483 compounds E6.1 to E6.483 of formula (Ia) wherein A^{2a} is CH, R⁵ is CH₃ and R², A³, R⁴, A¹, A^{2b} are as defined in table Z.

Table 1.7 provides 483 compounds E7.1 to E7.483 of formula (Ia) wherein A^{2a} is CH, R⁵ is OCH₃ and R², A³, R⁴, A¹, A^{2b} are as defined in table Z.

Table 1.8 provides 483 compounds E8.1 to E8.483 of formula (Ia) wherein A^{2a} is CH, R⁵ is cyclopropyl and R², A³, R⁴, A¹, A^{2b} are as defined in table Z.

Table 1.9 provides 483 compounds E9.1 to E9.483 of formula (Ia) wherein A^{2a} is CH, R⁵ is CH₂OCH₃ and R², A³, R⁴, A¹, A²⁶ are as defined in table Z.

Table 1.10 provides 483 compounds E10.1 to E10.483 of formula (Ia) wherein A^{2a} is CH, R⁵ is NHCH₂CH₃ and R², A³, R⁴, A¹, A²b are as defined in table Z.

Table 1.11 provides 483 compounds E11.1 to E11.483 of formula (Ia) wherein A^{2a} is CF, R⁵ is CH₃ and R², A³, R⁴, A¹, A^{2b} are as defined in table Z.

Table 1.12 provides 483 compounds E12.1 to E12.483 of formula (Ia) wherein A^{2a} is CF, R⁵ is OCH₃ and R², A³, R⁴, A¹, A^{2b} are as defined in table Z.

Table 1.13 provides 483 compounds E13.1 to E13.483 of formula (Ia) wherein A^{2a} is CF, R⁵ is cyclopropyl and R², A³, R⁴, A¹, A^{2b} are as defined in table Z.

Table 1.14 provides 483 compounds E14.1 to E14.483 of formula (Ia) wherein A^{2a} is CF, R⁵ is CH₂OCH₃ and R², A³, R⁴, A¹, A^{2b} are as defined in table Z.

Table 1.15 provides 483 compounds E15.1 to E15.483 of formula (Ia) wherein A^{2a} is CF, R⁵ is NHCH₂CH₃ and R², A³, R⁴, A¹, A²b are as defined in table Z.

Table 1.16 provides 483 compounds E16.1 to E16.483 of formula (Ia) wherein A^{2a} is CCl, R⁵ is CH₃ and R², A³, R⁴, A¹, A^{2b} are as defined in table Z.

Table 1.17 provides 483 compounds E17.1 to E17.483 of formula (Ia) wherein A^{2a} is CCl, R⁵ is OCH₃ and R², A³, R⁴, A¹, A^{2b} are as defined in table Z.

Table 1.18 provides 483 compounds E18.1 to E18.483 of formula (Ia) wherein A^{2a} is CCl, R⁵ is cyclopropyl and R², A³, R⁴, A¹, A^{2b} are as defined in table Z.

Table 1.19 provides 483 compounds E19.1 to E19.483 of formula (Ia) wherein A^{2a} is CCl, R⁵ is CH₂OCH₃ and R², A³, R⁴, A¹, A^{2b} are as defined in table Z.

Table 1.20 provides 483 compounds E20.1 to E20.483 of formula (Ia) wherein A^{2a} is CCl, R⁵ is NHCH₂CH₃ and R², A³, R⁴, A¹, A^{2b} are as defined in table Z.

Table 1.21 provides 483 compounds E21.1 to E21.483 of formula (Ia) wherein A^{2a} is CBr, R⁵ is CH₃ and R², A³, R⁴, A¹, A^{2b} are as defined in table Z.

Table 1.22 provides 483 compounds E22.1 to E22.483 of formula (Ia) wherein A^{2a} is CBr, R⁵ is OCH₃ and R², A³, R⁴, A¹, A^{2b} are as defined in table Z.

Table 1.23 provides 483 compounds E23.1 to E23.483 of formula (Ia) wherein A^{2a} is CBr, R⁵ is cyclopropyl and R², A³, R⁴, A¹, A^{2b} are as defined in table Z.

Table 1.24 provides 483 compounds E24.1 to E24.483 of formula (Ia) wherein A^{2a} is CBr, R⁵ is CH₂OCH₃ and R², A³, R⁴, A¹, A^{2b} are as defined in table Z.

Table 1.25 provides 483 compounds E25.1 to E25.483 of formula (Ia) wherein A^{2a} is CBr, R⁵ is NHCH₂CH₃ and R², A³, R⁴, A¹, A^{2b} are as defined in table Z.

Table 1.26 provides 483 compounds E26.1 to E26.483 of formula (Ia) wherein A^{2a} is CCH₃, R⁵ is CH₃ and R², A³, R⁴, A¹, A^{2b} are as defined in table Z.

Table 1.27 provides 483 compounds E27.1 to E27.483 of formula (Ia) wherein A^{2a} is CCH₃, R⁵ is OCH₃ and R², A³, R⁴, A¹, A²⁶ are as defined in table Z.

Table 1.28 provides 483 compounds E28.1 to E28.483 of formula (Ia) wherein A^{2a} is CCH₃, R⁵ is cyclopropyl and R², A³, R⁴, A¹, A^{2b} are as defined in table Z.

Table 1.29 provides 483 compounds E29.1 to E29.483 of formula (Ia) wherein A^{2a} is CCH₃, R⁵ is CH₂OCH₃ and R², A³, R⁴, A¹, A^{2b} are as defined in table Z.

Table 1.30 provides 483 compounds E30.1 to E30.483 of formula (Ia) wherein A^{2a} is CCH₃, R⁵ is NHCH₂CH₃ and R², A³, R⁴, A¹, A^{2b} are as defined in table Z.

Table 1.31 provides 483 compounds E31.1 to E31.483 of formula (Ia) wherein A^{2a} is CCH₂CH₃, R⁵ is CH₃ and R², A³, R⁴, A¹, A^{2b} are as defined in table Z.

Table 1.32 provides 483 compounds E32.1 to E32.483 of formula (Ia) wherein A^{2a} is CCH₂CH₃, R⁵ is OCH₃ and R², A³, R⁴, A¹, A^{2b} are as defined in table Z.

Table 1.33 provides 483 compounds E33.1 to E33.483 of formula (Ia) wherein A^{2a} is CCH₂CH₃, R⁵ is cyclopropyl and R², A³, R⁴, A¹, A^{2b} are as defined in table Z.

Table 1.34 provides 483 compounds E34.1 to E34.483 of formula (Ia) wherein A^{2a} is CCH₂CH₃, R⁵ is CH₂OCH₃ and R², A³, R⁴, A¹, A^{2b} are as defined in table Z.

Table 1.35 provides 483 compounds E35.1 to E35.483 of formula (Ia) wherein A^{2a} is CCH₂CH₃, R⁵ is NHCH₂CH₃ and R², A³, R⁴, A¹, A²b are as defined in table Z.

Table 1.36 provides 483 compounds E36.1 to E36.483 of formula (Ia) wherein A^{2a} is CCN, R⁵ is CH₃ and R², A³, R⁴, A¹, A^{2b} are as defined in table Z.

Table 1.37 provides 483 compounds E37.1 to E37.483 of formula (Ia) wherein A^{2a} is CCN, R⁵ is OCH₃ and R², A³, R⁴, A¹, A^{2b} are as defined in table Z.

Table 1.38 provides 483 compounds E38.1 to E38.483 of formula (Ia) wherein A^{2a} is CCN, R⁵ is cyclopropyl and R², A³, R⁴, A¹, A^{2b} are as defined in table Z.

Table 1.39 provides 483 compounds E39.1 to E39.483 of formula (Ia) wherein A^{2a} is CCN, R⁵ is CH₂OCH₃ and R², A³, R⁴, A¹, A^{2b} are as defined in table Z.

Table 1.40 provides 483 compounds E40.1 to E40.483 of formula (Ia) wherein A^{2a} is CCN, R⁵ is NHCH₂CH₃ and R², A³, R⁴, A¹, A²b are as defined in table Z.

Table 1.41 provides 483 compounds E41.1 to E41.483 of formula (Ia) wherein A^{2a} is COCH₃, R⁵ is CH₃ and R², A³, R⁴, A¹, A^{2b} are as defined in table Z.

Table 1.42 provides 483 compounds E42.1 to E42.483 of formula (Ia) wherein A^{2a} is COCH₃, R⁵ is OCH₃ and R², A³, R⁴, A¹, A^{2b} are as defined in table Z.

Table 1.43 provides 483 compounds E43.1 to E43.483 of formula (Ia) wherein A^{2a} is COCH₃, R⁵ is cyclopropyl and R², A³, R⁴, A¹, A^{2b} are as defined in table Z.

Table 1.44 provides 483 compounds E44.1 to E44.483 of formula (Ia) wherein A^{2a} is COCH₃, R⁵ is CH₂OCH₃ and R², A³, R⁴, A¹, A^{2b} are as defined in table Z.

Table 1.45 provides 483 compounds E45.1 to E45.483 of formula (Ia) wherein A^{2a} is COCH₃, R⁵ is NHCH₂CH₃ and R², A³, R⁴, A¹, A²b are as defined in table Z.

Compounds according to the invention may possess any number of benefits including, inter alia, advantageous levels of biological activity for protecting plants against diseases that are caused by fungi or superior properties for use as agrochemical active ingredients (for example, greater biological activity, an advantageous spectrum of activity, an increased safety profile, improved physico-chemical properties, or increased biodegradability). Compounds according to the invention have particularly advantageous levels of biological activity for protecting plants against oomycetes such as Phytophthora, Plasmopara and Pythium.

Compounds of formula (I), wherein Z is O, can be made as shown in the following schemes 1 to 13, in which, unless otherwise stated, the definition of each variable is as defined in the present invention.

Compounds of formula (I) can be prepared via Suzuki cross coupling of compounds of formula (II), wherein X is Cl, Br or I, and a compound of formula (III), wherein either R⁶ is independently from each other hydrogen, C₁₋₆alkyl or wherein two R⁶ together can form a C₃₋₆cycloalkyl, in the presence of a base, such as Cs₂CO₃, K₂CO₃ or NaOtBu, and a suitable palladium catalyst, such as tetrakistriphenylphosphinepalladium, [1,1-bis(diphenylphosphino)ferrocene]dichloropalladium(II), bis(diphenylphosphine)palladium(II) chloride, palladium dichloride or palladium acetate, in a suitable solvent, such as dimethylformamide, dioxane, tetrahydrofuran, ethanol or water. Compounds of formula (III), wherein either R⁶ is independently from each other hydrogen, C₁₋₆alkyl or wherein two R⁶ together can form a C₃₋₆cycloalkylare prepared by known methods or are commercially available. This transformation is depicted in Scheme 1.

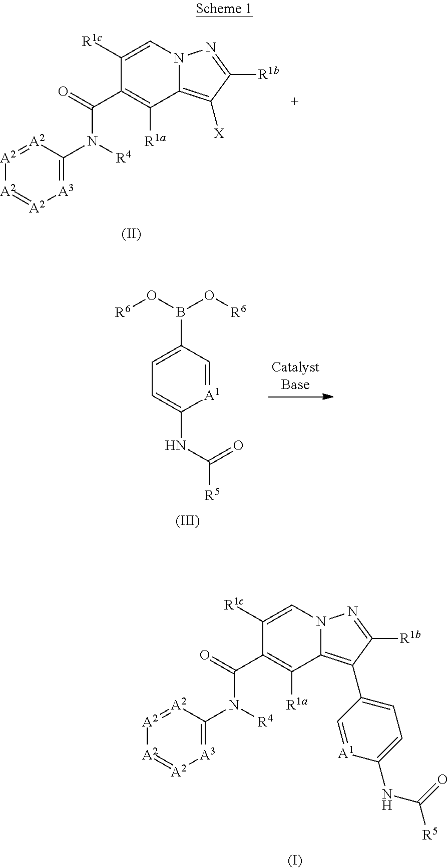

Compounds of formula (II), wherein X is Cl, Br or I, can be prepared by reacting compounds of formula (IV), wherein X is Cl, Br or I, with an a reagent of formula (V), wherein X¹ is a good leaving group such as Cl, Br, I, triflate, tosyl or mesyl, in the presence of a base such as CS₂CO₃, K₂CO₃, NaH or NaOtBu. A compound of formula (VI) wherein R⁷ is an electron-withdrawing group such as a cyano or an ester, can alternatively be used to functionalize the amide nitrogen, optionally in the presence of a base such as triethylamine or 1,8-diazabicyclo[5.4.0]undec-7-ene. Compounds of formula (IV), wherein X is Cl, Br or I, can be prepared by a peptide-coupling reaction between a compound of formula (VII), wherein X² is OH, and an amine of formula (VIII), and by activating the carboxylic acid function of the compounds of formula (VII), a process that usually takes place by converting the —OH of the carboxylic acid into a good leaving group, such as a chloride group, for example by using (COCl)₂ or SOCl₂, prior to treatment with amines of formula (VIII), preferably in a suitable solvent (e.g., N-methylpyrrolidone, acetonitrile, dimethylacetamide, dichloromethane or tetrahydrofuran), preferably at temperatures between 25° C. and 60° C., and optionally in the presence of a base such as triethylamine or N,N-diisopropylethylamine; or alternatively under conditions described in the literature for an amide coupling such as 1-propanephosphonic acid cyclic anhydride (T3P) in suitable solvent (e.g., acetonitrile) optionally in the pr-esence of a base (e.g., triethylamine or N,N-diisopropylethylamine). For examples, see Chem. Soc. Rev. 2009, 38, 606 and Chem. Soc. Rev. 2011, 40, 5084. Alternatively, compounds of formula (II), wherein X is Cl, Br or I, can be obtained by an amide-coupling transformation between compounds of formula (VII), wherein X² is OH, and amines of formula (IX) using the conditions described above. These transformations are depicted in Scheme 2.

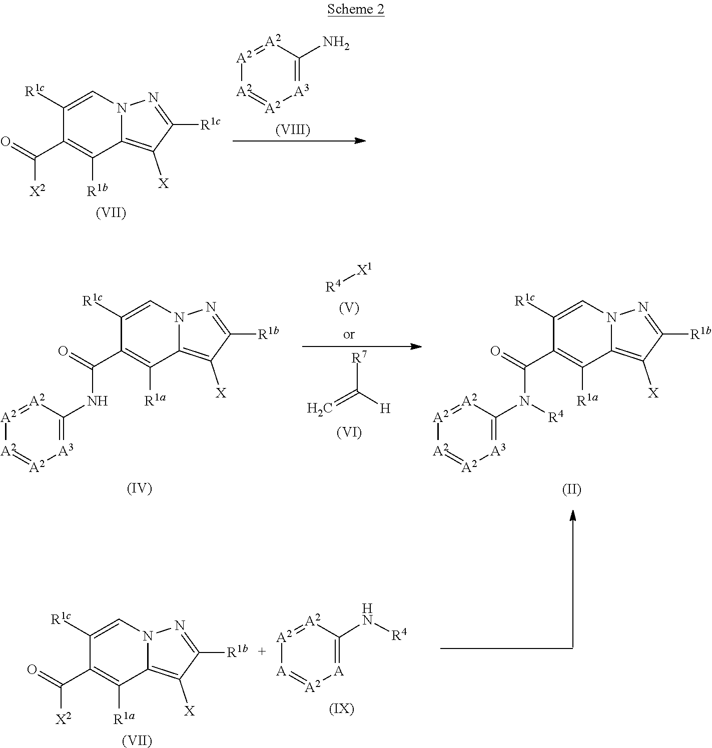

Compounds of formula (IX) are commercially available or can be obtained by reaction of compounds of formula (VIII) with a compound of formula (V), wherein X¹ is a good leaving group such as Cl, Br, I, triflate, tosyl or mesyl, in the presence of a base such as Cs₂CO₃, K₂CO₃, NaH or NaOtBu. Alternatively, compounds of formula (IX) can also be synthesized by reacting compounds of formula (VIII) with compounds of formula (VI), wherein R⁷ is an electron-withdrawing group such as cyano or ester, optionally in the presence of a base such as triethylamine or 1,8-diazabicyclo[5.4.0]undec-7-ene. Alternatively, compounds of formula (IX) can be obtained from amines of formula (VIII) by a reductive amination with aldehydes of formula (X), wherein R⁸ is selected from C₁₋₆alkyl, C₁₋₆alkoxy-C₁₋₆alkyl, C₃₋₆ cycloalkyl-C₁₋₃alkyl, C₁₋₆alkenyl, C₁₋₆alkynyl, C₁₋₆alkoxy, C₁₋₆alkylsulfanyl-C₁₋₅alkyl, C₁₋₆alkylsulfinyl-C₁₋₅alkyl, C₁₋₆alkylsulfonyl-C₁₋₅alkyl, C₁₋₆alkoxycarbonyl-C₁₋₅alkyl, C₁₋₆alkylaminocarbonyl-C₁₋₅alkyl, or diC₁₋₆alkylaminocarbonyl-C₁₋₆alkyl, e.g. in the presence of NaBH(OAc)₃ or NaBH₃CN, in a suitable solvent, optionally in the presence of a Brönsted or Lewis acid such as acetic acid. Alternatively, another reagent system for the reductive amination uses a combination of titanium isopropoxide and NaBH₄. The synthesis of compounds of formula (IX) from amines of formula (VIII) is depicted in Scheme 3.

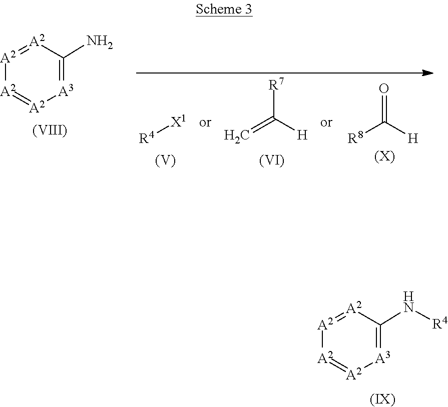

Alternatively, compounds of formula (II), wherein X is Cl, Br or I, can be prepared from the reaction of a compound of formula (XI) and a halogenating agent, such as N-chlorosuccinimide, N-bromosuccinimide, N-iodosuccinimide or bromine in a suitable solvent, such as dichloromethane, chloroform, tetrahydrofuran or acetonitrile. Compounds of formula (XI) can be obtained by an amide-coupling transformation of amines of formula (IX) with compounds of formula (XII), wherein X² is OH, and by activating the carboxylic acid function of the compounds of formula (XII), a process that usually takes place by converting the —OH of the carboxylic acid into a good leaving group, such as a chloride group, for example by using (COCl)₂ or SOCl₂, prior to treatment with amines of formula (IX), preferably in a suitable solvent (e.g., N-methylpyrrolidone, acetonitrile, dimethylacetamide, dichloromethane or tetrahydrofuran), preferably at temperatures between 25° C. and 60° C., and optionally in the presence of a base such as triethylamine or N,N-diisopropylethylamine; or alternatively under conditions described in the literature for an amide coupling such as 1-propanephosphonic acid cyclic anhydride (T3P) in suitable solvent (e.g., acetonitrile) optionally in the presence of a base (e.g., triethylamine or N,N-diisopropylethylamine). Alternatively, compounds of formula (XI) can be obtained by alkylation of compounds of formula (XIII) with compounds of formula (V), wherein X¹ is a good leaving group such as Cl, Br, I, triflate, tosyl or mesyl, in the presence of a base such as Cs₂CO₃, K₂CO₃, NaH or NaOtBu. Alternatively, compounds of formula (XI) can also be synthesized by reacting compounds of formula (XIII) with compounds of formula (VI), wherein R⁷ is an electron-withdrawing group such as cyano or ester, optionally in the presence of a base such as triethylamine or 1,8-diazabicyclo[5.4.0]undec-7-ene. Compounds of formula (XIII) can be prepared by a peptide-coupling transformation from compounds of formula (XII), wherein X² is OH, with amines of formula (VIII) using the conditions described above. These transformations are depicted in Scheme 4.

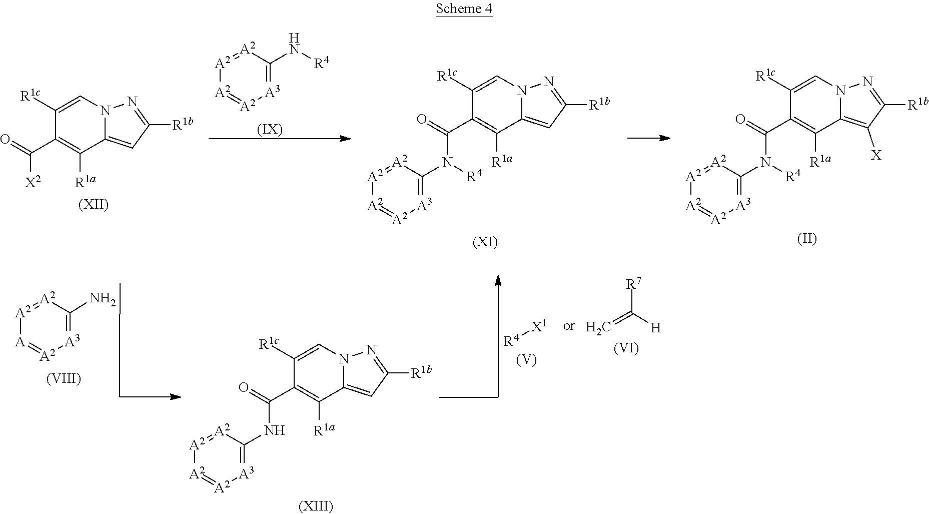

Alternatively, compounds of formula (XI) can be synthesized by reacting compounds of formula (XIV), wherein X is Cl, Br or I, with amines of formula (IX) and carbon monoxide in the presence of a catalyst such as [1,1′-bis(diphenylphosphino)ferrocene]dichloropalladium(II), and, optionally, a base such as triethylamine. This transformation is depicted in Scheme 5.

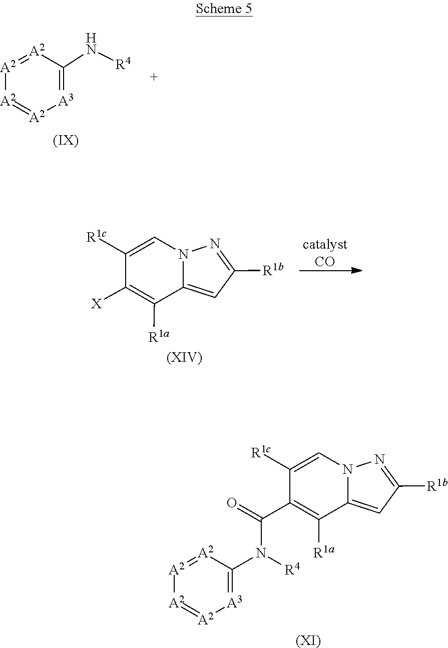

Compounds of formula (XII), wherein X² is OH, are commercially available or can be obtained by decarboxylation of compounds of formula (XV), wherein R⁹ is C₁₋₆alkyl, obtained by hydrolyzing the ester moiety under conventional acidic or basic conditions and heating up the di-acid. Compounds of formula (XV), wherein R⁹ is C₁₋₆alkyl, can be synthesized by reaction of compounds of formula (XVI), wherein R¹⁰ is an (aryl)sulfonyl, (hetero)arylsulfonyl or a sulfonic acid group, for instance a sulfonic acid, tosylate or 2,4,6-trimethylbenzenesulfonyl group, with alkynes of formula (XVII), wherein R⁹ is C₁₋₆alkyl, in the presence of a base such as Na₂CO₃ or K₂CO₃ in a polar organic solvent such as dimethylsulfoxide, N-methylpyrrolidine or N,N-dimethylformamide. Compounds of formula (XVII) are commercially available or can be easily prepared by those skilled in the art following described methods. Compounds of formula (XVI), wherein R¹⁰ is an (aryl)sulfonyl, (hetero)arylsulfonyl or a sulfonic acid group, for instance a sulfonic acid, tosylate or 2,4,6-trimethylbenzenesulfonyl group, can be formed by reacting compounds of formula (XVIII) with compounds of formula (XIX), wherein R¹⁰ is an (aryl)sulfonyl, (hetero)arylsulfonyl or a sulfonic acid group, for instance a sulfonic acid, tosylate or 2,4,6-trimethylbenzenesulfonyl group, in a polar aprotic solvent such as dimethylsulfoxide, N-methylpyrrolidine or N,N-dimethylformamide. Compounds of formula (XVIII) are commercially available or can be prepared by methods known to those skilled in the art. Compounds of formula (XII), wherein X² is OH, can be further reacted with a C₁₋₆alkylalcohol to give esters of formula (XX), wherein R¹¹ is C₁₋₆alkyl. These transformations are depicted in Scheme 6.

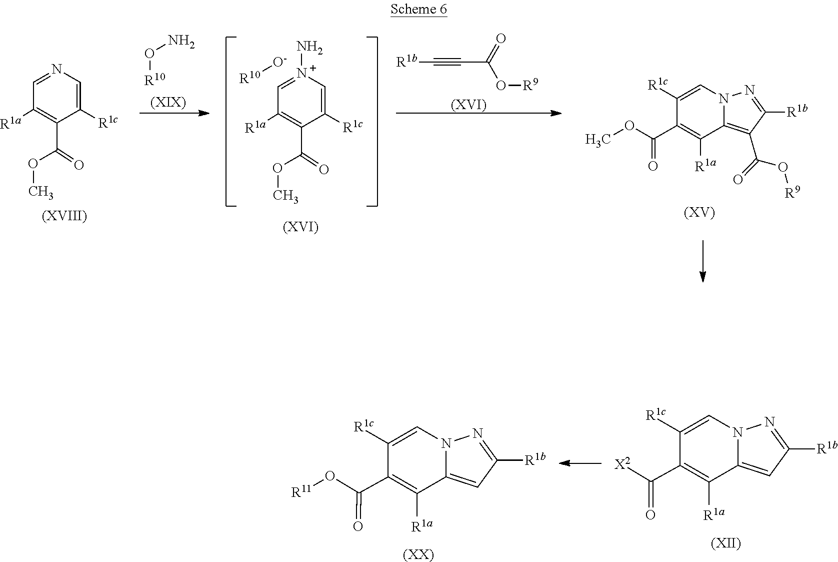

Alternatively, compounds of formula (I) can be prepared by an amide-coupling transformation of amines of formula (IX) with compounds of formula (XXI), wherein X is Cl, Br or I, and X² is OH, and by activating the carboxylic acid function of the compounds of formula (XXI), a process that usually takes place by converting the —OH of the carboxylic acid into a good leaving group, such as a chloride group, for example by using (COCl)₂ or SOCl₂, prior to treatment with amines of formula (IX), preferably in a suitable solvent (e.g., N-methylpyrrolidone, acetonitrile, dimethylacetamide, dichloromethane or tetrahydrofuran), preferably at temperatures between 25° C. and 60° C., and optionally in the presence of a base such as triethylamine or N,N-diisopropylethylamine; or alternatively under conditions described in the literature for an amide coupling such as 1-propanephosphonic acid cyclic anhydride (T3P) in suitable solvent (e.g., acetonitrile) optionally in the presence of a base (e.g., triethylamine or N,N-diisopropylethylamine). Compounds of formula (I) can alternatively be prepared by alkylation of compounds of formula (XXII) with compounds of formula (V), wherein X¹ is a good leaving group such as Cl, Br, I, triflate, tosyl or mesyl, in the presence of a base such as Cs₂CO₃, K₂CO₃, NaH or NaOtBu. Alternatively, compounds of formula (I) can also be synthesized by reacting compounds of formula (XXII) with compounds of formula (VI), wherein R⁷ is an electron-withdrawing group such as cyano or ester, optionally in the presence of a base such as triethylamine or 1,8-diazabicyclo[5.4.0]undec-7-ene. Compounds of formula (XXII) can be synthesized by a peptide-coupling transformation from compounds of formula (XXI), wherein X² is OH, and amines of formula (Vill) using the conditions described above. These transformations are depicted in Scheme 7.

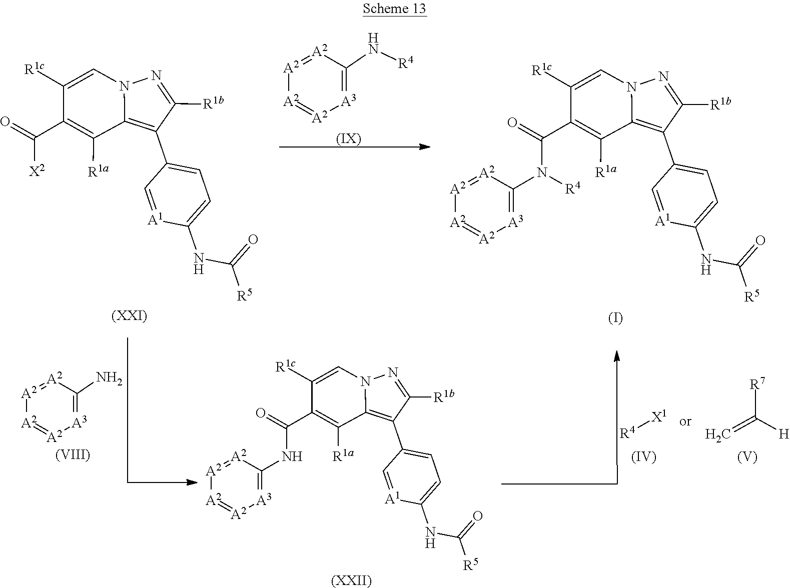

Compounds of formula (XXI), wherein X² is OH, can be obtained by hydrolysis of the ester moiety of compounds of formula (XXIII), wherein R¹¹ is C₁₋₆alkyl. Compounds of formula (XXIII), wherein R¹¹ is C₁₋₆alkyl, can result from a Suzuki cross-coupling reaction between compounds of formula (XXIV), wherein X is Cl, Br or I, and R¹⁰ is C₁₋₆alkyl, and compounds of formula (III), wherein either R⁶ is independently from each other hydrogen, C₁₋₆alkyl or wherein two R⁶ together can form a C₃₋₆-acycloalkyl, in the presence of a base, such as Cs₂CO₃, K₂CO₃ or NaOtBu, and a suitable palladium catalyst, such as tetrakistriphenylphosphinepalladium, [1,1-bis(diphenylphosphino)ferrocene]dichloropalladium(II), bis(diphenylphosphine)palladium(II) chloride, palladium dichloride or palladium acetate, in a suitable solvent, such as dimethylformamide, dioxane, tetrahydrofuran, ethanol or water. Compounds of formula (XXIV), wherein X is Cl, Br or I, and R¹¹ is C₁₋₆alkyl, can be synthesized by the reaction of a compound of formula (XX), wherein R¹¹ is C₁₋₆alkyl, and a halogenating agent, such as N-chlorosuccinimide, N-bromosuccinimide, N-iodosuccinimide or bromine in a suitable solvent, such as dichloromethane, chloroform, tetrahydrofuran or acetonitrile. These transformations are depicted in Scheme 8.

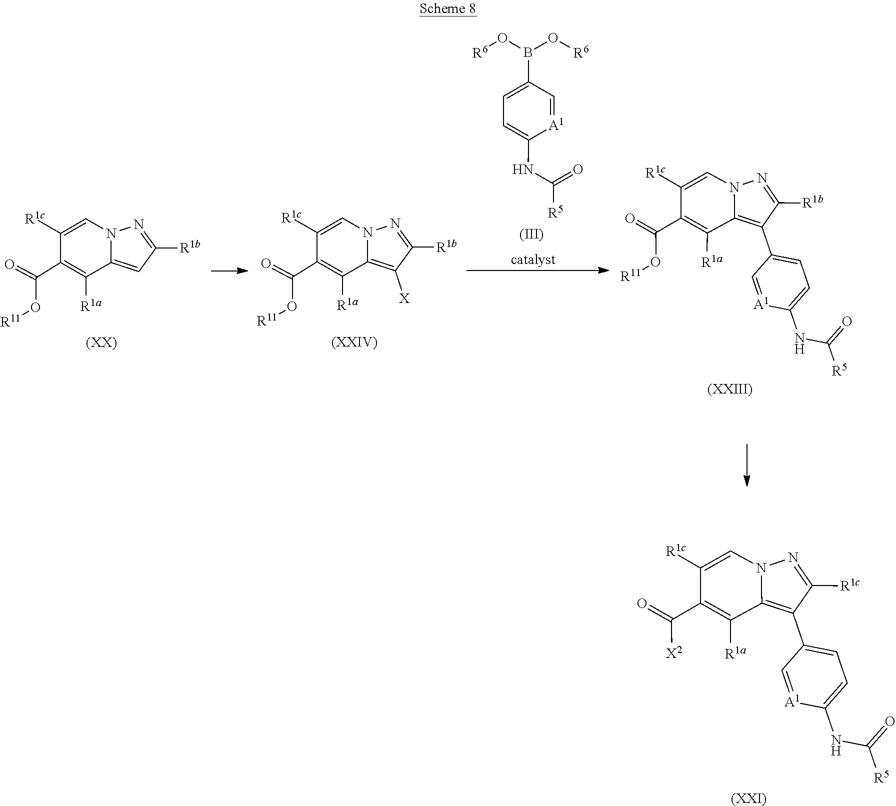

Alternatively, compounds of formula (I) can be synthesized by reacting compounds of formula (XXV), wherein X is Cl, Br or I, with amines of formula (IX) and carbon monoxide in the presence of a catalyst such as [1,1-bis(diphenylphosphino)ferrocene]dichloropalladium(II), and, optionally, a base such as triethylamine. Compounds of formula (XXV), wherein X is Cl, Br or I, can be prepared from compounds of formula (XXVI), wherein either X is independently from each other Cl, Br or I, through a Suzuki cross-coupling with compounds of formula (III), wherein either R⁵ is independently from each other hydrogen, C₁₋₆alkyl or wherein two R⁶ together can form a C₃₋₆cycloalkyl, in the presence of a base, such as Cs₂CO₃, K₂CO₃ or NaOtBu, and a suitable palladium catalyst, such as tetrakistriphenylphosphinepalladium, [1,1-bis(diphenylphosphino)ferrocene]dichloropalladium(II), bis(diphenylphosphine)palladium(II) chloride, palladium dichloride or palladium acetate, in a suitable solvent, such as dimethylformamide, dioxane, tetrahydrofuran, ethanol or water. These transformations are depicted in Scheme 9.

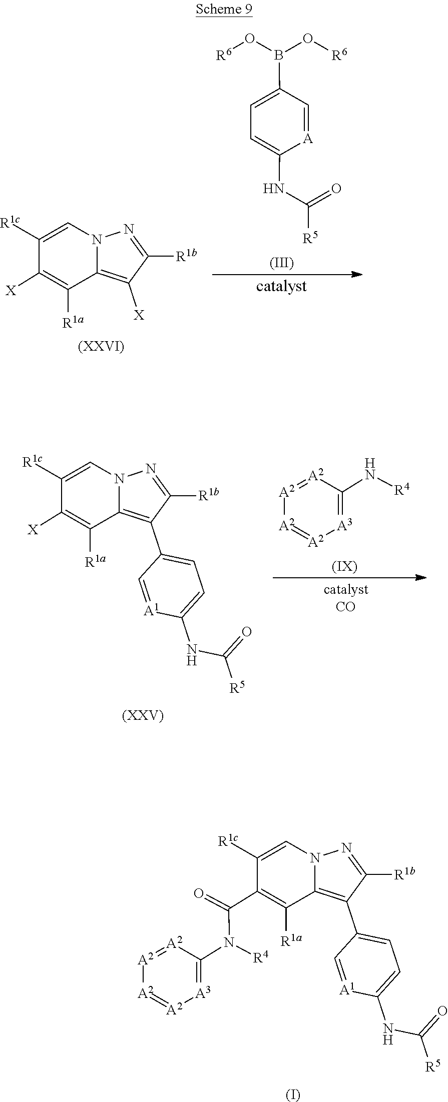

Compounds of formula (XXIII), wherein R¹¹ is C₁₋₆alkyl, can be synthesized by reaction of compounds of formula (XXVII), wherein X is Cl, Br or I, R¹¹ is C₁₋₆alkyl, and R¹⁰ is an (aryl)sulfonyl, (hetero)arylsulfonyl or a sulfonic acid group, for instance a sulfonic acid, tosylate or 2,4,6-trimethylbenzenesulfonyl group, with alkynes of formula (XXVIII) in the presence of a base such as Na₂CO₃ or K₂CO₃ in a polar organic solvent such as dimethylsulfoxide, N-methylpyrrolidine or N,N-dimethylformamide. Compounds of formula (XXVIII) are commercially available or can be easily prepared by those skilled in the art following described methods. Compounds of formula (XXVII), wherein X is Cl, Br or I, R¹¹ is C₁₋₆alkyl, and R¹⁰ is an (aryl)sulfonyl, (hetero)arylsulfonyl or a sulfonic acid group, for instance a sulfonic acid, tosylate or 2,4,6-trimethylbenzenesulfonyl group, can be formed by reacting compounds of formula (XXIX), wherein X is Cl, Br or I, and R¹¹ is C₁₋₆alkyl, with compounds of formula (XIX), wherein R¹⁰ is an (aryl)sulfonyl, (hetero)arylsulfonyl or a sulfonic acid group, for instance a sulfonic acid, tosylate or 2,4,6-trimethylbenzenesulfonyl group, in a polar aprotic solvent such as dimethylsulfoxide, N-methylpyrrolidine or N,N-dimethylformamide. Compounds of formula (XXIX) are commercially available or can be prepared by methods known to those skilled in the art. These transformations are depicted in Scheme 10.

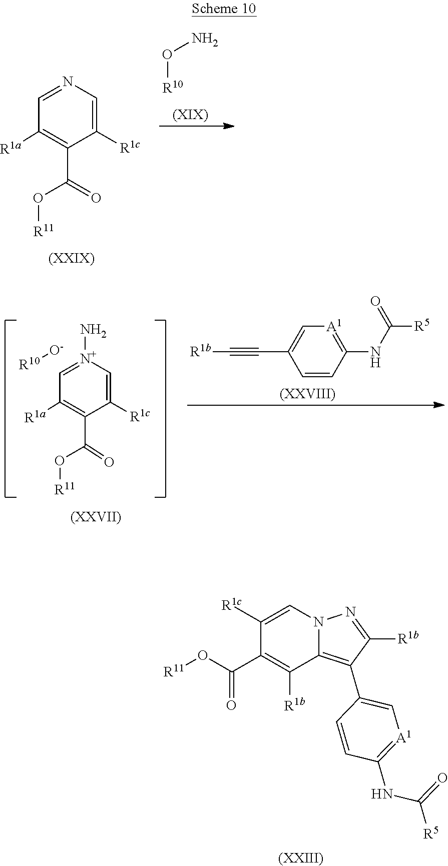

Alternatively, compounds of formula (I) can be prepared by the reaction of a compound of formula (XXX) with compounds of formula (XXXI), wherein X² is OH, in a peptide-coupling transformation, using the conditions described above. Compounds of formula (XXXI) can be prepared via Suzuki cross coupling of a compound of formula (II) and a compound of formula (XXXII), wherein either R⁶ is independently from each other hydrogen, C₁₋₆alkyl or wherein two R⁶ together can form a C₃₋₆cycloalkyl, in the presence of a base, such as Cs₂CO₃, K₂CO₃ or NaOtBu, and a suitable palladium catalyst, such as tetrakistriphenylphosphinepalladium, [1,1-bis(diphenylphosphino)ferrocene]dichloropalladium(II), bis(diphenylphosphine)palladium(II) chloride, palladium dichloride or palladium acetate, in a suitable solvent, such as dimethylformamide, dioxane, tetrahydrofuran, ethanol or water. Compounds of formula (XXXII), wherein either R⁶ is independently from each other hydrogen, C₁₋₆alkyl or wherein two R⁶ together can form a C₃₋₆cycloalkyl are prepared by known methods or are commercially available. These transformations are depicted in Scheme 11.

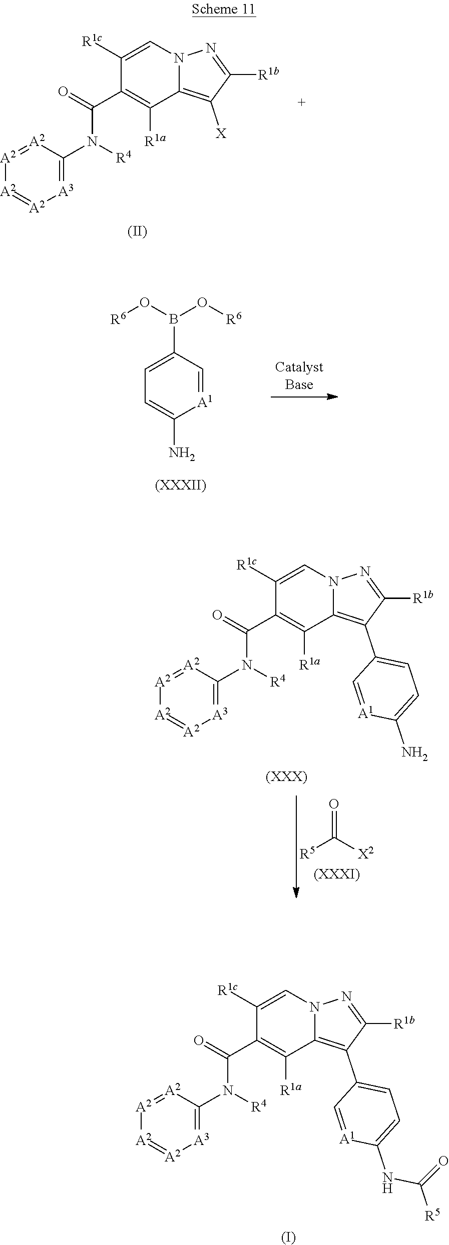

Compounds of formula (Ib), wherein Z is S, can be prepared by the reaction of a compound of formula (I), wherein Z is O, with phosphorus pentasulfide or Lawesson's reagent (CAS: 19172-47-5) in a suitable solvent such as toluene, xylene or dichloromethane. This transformation is depicted in Scheme 12.

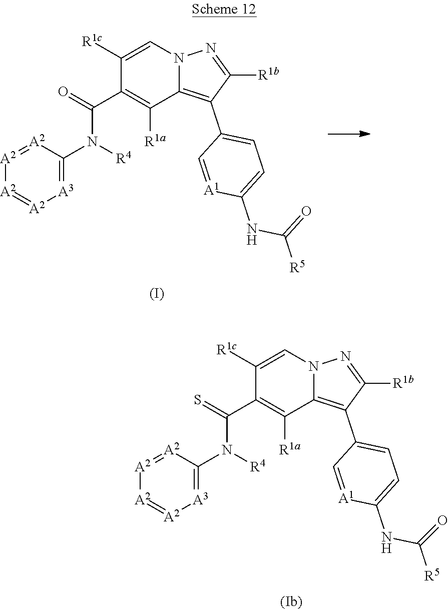

Alternatively, compounds of formula (Ib), wherein Z is S, can be prepared by the reaction of a compound of formula (XXXb) with compounds of formula (XXXI), wherein X² is OH, in a peptide-coupling transformation, using the conditions described above. Compounds of formula (XXXb) can be prepared by the reaction of a compound of formula (XXX), with phosphorus pentasulfide or Lawesson's reagent (CAS: 19172-47-5) in a suitable solvent such as toluene, xylene or dichloromethane. This transformation is depicted in Scheme 13.

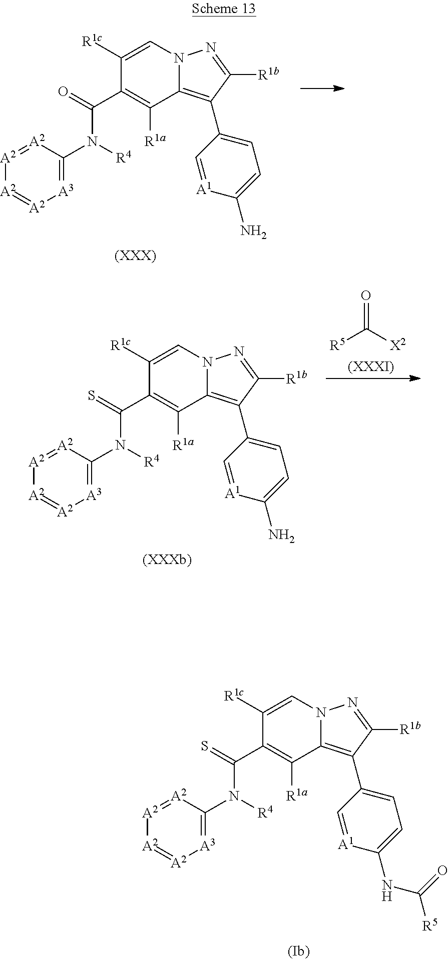

It is understood by the person skilled in the art that the amide coupling reactions described above between an acid, an amine and a coupling agent could also be performed using the corresponding acid chloride and amine. The transformation of an acid into its corresponding acid chloride is well known by the person skilled in the art.

When the term “compound/compounds according to the invention” is used, then this refers to compounds according to the present invention.

Alternatively, the compounds according to the present invention can be obtained by using standard synthesis techniques known to the person skilled in the art. Non-exhaustive examples include oxidation reactions, reduction reactions, hydrolysis reactions, coupling reactions, aromatic nucleophilic or electrophilic substitution reactions, nucleophilic substitution reactions, nucleophilic addition reactions, olefination reactions, oxime formation, alkylation and halogenation reactions.

A compound according to the present invention can be converted in a manner known per se into another compound according to the present invention by replacing one or more substituents of the starting compound according to the present invention in the customary manner by (an) other substituent(s) according to the invention.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of the compounds according to the present invention can be prepared in a manner known per se. Thus, for example, acid addition salts of the compounds according to the present invention are obtained by treatment with a suitable acid or a suitable ion exchanger reagent and salts with bases are obtained by treatment with a suitable base or with a suitable ion exchanger reagent.

Salts of compounds the compounds according to the present invention can be converted in the customary manner into the free compounds, acid addition salts, for example, by treatment with a suitable basic compound or with a suitable ion exchanger reagent and salts with bases, for example, by treatment with a suitable acid or with a suitable ion exchanger reagent.

Salts of the compounds according to the present invention can be converted in a manner known per se into other salts of the compounds according to the present invention, acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds according to the present invention, which have salt-forming properties can be obtained in free form or in the form of salts.

The compounds according to the present invention and, where appropriate, the tautomers thereof, in each case in free form or in salt form, can be present in the form of one of the stereoisomers which are possible or as a mixture of these, for example in the form of pure stereoisomers, such as antipodes and/or diastereomers, or as stereoisomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure stereoisomers and also to all stereoisomer mixtures which are possible and is to be understood in each case in this sense hereinabove and hereinbelow, even when stereochemical details are not mentioned specifically in each case.

Diastereomer mixtures or racemate mixtures of the compounds according to the present invention, in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable stereoisomer mixtures, but also by generally known methods of diastereoselective or enantioselective synthesis, for example by carrying out the process according to the invention with starting materials of a suitable stereochemistry.

N-oxides can be prepared by reacting a compound according to the present invention with a suitable oxidizing agent, for example the H2O2/urea adduct in the presence of an acid anhydride, e.g. trifluoroacetic anhydride. Such oxidations are known from the literature, for example from J. Med. Chem., 32 (12), 2561-73, 1989 or WO 00/15615.

It is advantageous to isolate or synthesize in each case the biologically more effective stereoisomer, for example enantiomer or diastereomer, or stereoisomer mixture, for example enantiomer mixture or diastereomer mixture, if the individual components have a different biological activity.

The compounds according to the present invention and, where appropriate, the tautomers thereof, in each case in free form or in salt form, can, if appropriate, also be obtained in the form of hydrates and/or include other solvents, for example those which may have been used for the crystallization of compounds which are present in solid form.

The following Examples illustrate, but do not limit, the invention.

The present invention also provides intermediates useful for the preparation of compounds according to the present invention.

The below intermediate forms a further aspect of the invention.

A compound of formula (XI)

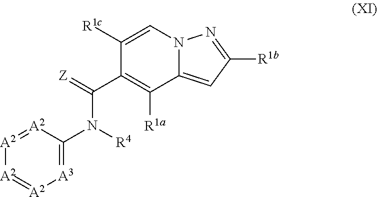

wherein Z is O or S, and preferably Z is O;
R^{1a}, R^{1b} and R^{1c} are independently selected from hydrogen, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl; and preferably R^{1a}, R^{1b} and R^{1c} are hydrogen; A² are independently CR² or N, with the proviso that no more than three A² are N, preferably no more than two A² are N, preferably no more than one A² is N, and more preferably the four A² are CR²; R² are independently selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl, wherein each of the C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl groups is optionally substituted with one substituent independently selected from halogen, hydroxy, and CN; and preferably R² are independently selected from hydrogen, halogen, CN, C₁₋₆alkyl, C₁₋₆alkoxycarbonyl, and C₁₋₆alkoxy; with the proviso that at least two R² are preferably not hydrogen; A³ is independently CR³ or N;
R³ is selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl, wherein each of the C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl groups is optionally substituted with one substituent independently selected from halogen, hydroxy, and CN; and preferably R³ is hydrogen;
R⁴ is selected from C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, C₁₋₆alkoxy, C₁₋₆alkylsulfanyl-C₁₋₆alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl, diC₁₋₆alkylaminocarbonyl-C₁₋₆alkyl, and CN, wherein each of the C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, C₁₋₆alkoxy, C₁₋₆alkylsulfanyl-C₁₋₆alkyl, C₁₋₆alkylsulfinyl-C₁₋₆alkyl, C₁₋₆alkylsulfonyl-C₁₋₆alkyl, C₁₋₆ alkoxycarbonyl-C₁₋₆alkyl, C₁₋₆alkylaminocarbonyl-C₁₋₆alkyl and diC₁₋₆alkylaminocarbonyl-C₁₋₆alkyl groups is optionally substituted with one to three substituents independently selected from halogen and CN; and
wherein A³ and R⁴ taken together optionally form a ring, preferably a 5-8-membered heterocycle, and more preferably a 6-membered heterocycle;
or a salt or N-oxide thereof.

The compounds of formula (I) as defined in the present invention can be used in the agricultural sector and related fields of use e.g. as active ingredients for controlling plant pathogens or on non-living materials for control of spoilage microorganisms or organisms potentially harmful to man. The novel compounds are distinguished by excellent activity at low rates of application, by being well tolerated by plants and by being environmentally safe. They have very useful curative, preventive and systemic properties and may be used for protecting numerous cultivated plants. The compounds of formula (I) as defined in the present invention can be used to inhibit or destroy the pathogens that occur on plants or parts of plants (fruit, blossoms, leaves, stems, tubers, roots) of different crops of useful plants, while at the same time protecting also those parts of the plants that grow later e.g. from phytopathogenic microorganisms.

It is also possible to use compounds of formula (I) as defined in the present invention as fungicide. The term “fungicide” as used herein means a compound that controls, modifies, or prevents the growth of fungi. The term “fungicidally effective amount” means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

It is also possible to use compounds of formula (I) as defined in the present invention as dressing agents for the treatment of plant propagation material, e.g., seed, such as fruits, tubers or grains, or plant cuttings (for example rice), for the protection against fungal infections as well as against phytopathogenic fungi occurring in the soil. The propagation material can be treated with a composition comprising a compound of formula (I) as defined in the present invention before planting: seed, for example, can be dressed before being sown. The compounds of formula (I) as defined in the present invention can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

Furthermore the compounds of formula (I) as defined in the present invention can be used for controlling fungi in related areas, for example in the protection of technical materials, including wood and wood related technical products, in food storage, in hygiene management.

In addition, the invention could be used to protect non-living materials from fungal attack, e.g. lumber, wall boards and paint.

Compounds of formula (I) as defined in the present invention and fungicidal compositions containing them may be used to control plant diseases caused by a broad spectrum of fungal plant pathogens.

They are effective in controlling a broad spectrum of plant diseases, such as foliar pathogens of ornamental, turf, vegetable, field, cereal, and fruit crops.

These fungi and fungal vectors of disease, as well as phytopathogenic bacteria and viruses, which may be controlled are for example:
Absidia corymbifera, Alternaria spp, Aphanomyces spp, Ascochyta spp, Aspergillus spp. including A. flavus, A. fumigatus, A. nidulans, A. niger, A. terrus, Aureobasidium spp. including A. pullulans, Blastomyces dermatitidis, Blumeria graminis, Bremia lactucae, Botryosphaeria spp. including B. dothidea, B. obtusa, Botrytis spp. inclusing B. cinerea, Candida spp. including C. albicans, C. glabrata, C. krusei, C. lusitaniae, C. parapsilosis, C. tropicalis, Cephaloascus fragrans, Ceratocystis spp, Cercospora spp. including C. arachidicola, Cercosporidium personatum, Cladosporium spp, Claviceps purpurea,
Coccidioides immitis, Cochliobolus spp, Colletotrichum spp. including C. musae,
Cryptococcus neoformans, Diaporthe spp, Didymella spp, Drechslera spp, Elsinoe spp,
Epidermophyton spp, Erwinia amylovora, Erysiphe spp. including E. cichoracearum,
Eutypa lata, Fusarium spp. including F. culmorum, F. graminearum, F. langsethiae, F. moniliforme, F. oxysporum, F. proliferatum, F. subglutinans, F. solani, Gaeumannomyces graminis, Gibberella fujikuroi, Gloeodes pomigena, Gloeosporium musarum, Glomerella cingulate, Guignardia bidwellii, Gymnosporangium juniperi-virginianae, Helminthosporium spp, Hemileia spp, Histoplasma spp. including H. capsulatum, Laetisaria fuciformis, Leptographium lindbergi, Leveillula taurica, Lophodermium seditiosum, Microdochium nivale, Microsporum spp, Monilinia spp, Mucor spp, Mycosphaerella spp. including M. graminicola, M. pomi, Oncobasidium theobromaeon, Ophiostoma piceae, Paracoccidioides spp, Penicillium spp. including P. digitatum, P. italicum, Petriellidium spp, Peronosclerospora spp. Including P. maydis, P. philippinensis and P. sorghi, Peronospora spp, Phaeosphaeria nodorum, Phakopsora pachyrhizi, Phellinus igniarus, Phialophora spp, Phoma spp, Phomopsis viticola, Phytophthora spp. including P. infestans, Plasmopara spp. including P. halstedii, P. viticola, Pleospora spp., Podosphaera spp. including P. leucotricha, Polymyxa graminis, Polymyxa betae, Pseudocercosporella herpotrichoides, Pseudomonas spp, Pseudoperonospora spp. including P. cubensis, P. humuli, Pseudopeziza tracheiphila, Puccinia Spp. including P. hordei, P. recondita, P. striiformis, P. triticina, Pyrenopeziza spp, Pyrenophora spp, Pyricularia spp. including P. oryzae, Pythium spp. including P. ultimum, Ramularia spp, Rhizoctonia spp, Rhizomucor pusillus, Rhizopus arrhizus, Rhynchosporium spp, Scedosporium spp. including S. apiospermum and S. prolificans, Schizothyrium pomi,
Sclerotinia spp, Sclerotium spp, Septoria spp, including S. nodorum, S. tritici, Sphaerotheca macularis, Sphaerotheca fusca (Sphaerotheca fuliginea), Sporothorix spp, Stagonospora nodorum, Stemphylium spp,. Stereum hirsutum, Thanatephorus cucumeris, Thielaviopsis basicola, Tilletia spp, Trichoderma spp. including T. harzianum, T. pseudokoningii, T. viride,
Trichophyton spp, Typhula spp, Uncinula necator, Urocystis spp, Ustilago spp, Venturia spp. including V. inaequalis, Verticillium spp, and Xanthomonas spp.

In particular, compounds of formula (I) as defined in the present invention and fungicidal compositions containing them may be used to control plant diseases caused by a broad spectrum of fungal plant pathogens in the Basidiomycete, Ascomycete, Oomycete and/or Deuteromycete, Blasocladiomycete, Chrytidiomycete, Glomeromycete and/or Mucoromycete classes. More particularly, the compounds of formula (I) as defined in the present invention may be used to conrol oomycetes.

These pathogens may include:
Oomycetes, including Phytophthora diseases such as those caused by Phytophthora capsici, Phytophthora infestans, Phytophthora sojae, Phytophthora fragariae, Phytophthora nicotianae, Phytophthora cinnamomi, Phytophthora citricola, Phytophthora citrophthora and Phytophthora erythroseptica; Pythium diseases such as those caused by Pythium aphanidermatum, Pythium arrhenomanes, Pythium graminicola, Pythium irregulare, Pythium sylvaticum and Pythium ultimum; diseases caused by Peronosporales such as Peronospora destructor, Peronospora parasitica, Plasmopara viticola, Plasmopara halstedii, Pseudoperonospora cubensis, Albugo candida, Sclerophthora macrospora and Bremia lactucae; and others such as Aphanomyces cochlioides, Labyrinthula zosterae, Peronosclerospora sorghi and Sclerospora graminicola.

Ascomycetes, including blotch, spot, blast or blight diseases and/or rots for example those caused by Pleosporales such as Stemphylium solani, Stagonospora tainanensis, Spilocaea oleaginea, Setosphaeria turcica, Pyrenochaeta lycoperisici, Pleospora herbarum, Phoma destructiva, Phaeosphaeria herpotrichoides, Phaeocryptocus gaeumannii, Ophiosphaerella graminicola, Ophiobolus graminis, Leptosphaeria maculans, Hendersonia creberrima, Helminthosporium triticirepentis, Setosphaeria turcica, Drechslera glycines, Didymella bryoniae, Cycloconium oleagineum, Corynespora cassiicola, Cochliobolus sativus, Bipolaris cactivora, Venturia inaequalis, Pyrenophora teres, Pyrenophora tritici-repentis, Alternaria alternata, Alternaria brassicicola, Alternaria solani and Alternaria tomatophila, Capnodiales such as Septoria tritici, Septoria nodorum, Septoria glycines, Cercospora arachidicola, Cercospora sojina, Cercospora zeae-maydis, Cercosporella capsellae and Cercosporella herpotrichoides, Cladosporium carpophilum, Cladosporium effusum, Passalora fulva, Cladosporium oxysporum, Dothistroma septosporum, Isariopsis clavispora, Mycosphaerella fijiensis, Mycosphaerella graminicola, Mycovellosiella koepkeii, Phaeoisariopsis bataticola, Pseudocercospora vitis, Pseudocercosporella herpotrichoides, Ramularia beticola, Ramularia collo-cygni, Magnaporthales such as Gaeumannomyces graminis, Magnaporthe grisea, Pyricularia oryzae, Diaporthales such as Anisogramma anomala, Apiognomonia errabunda, Cytospora platani, Diaporthe phaseolorum, Discula destructiva, Gnomonia fructicola, Greeneria uvicola, Melanconium juglandinum, Phomopsis viticola, Sirococcus clavigignenti-juglandacearum, Tubakia dryina, Dicarpella spp., Valsa ceratosperma, and others such as Actinothyrium graminis, Ascochyta pisi, Aspergillus flavus, Aspergillus fumigatus, Aspergillus nidulans, Asperisporium caricae, Blumeriella jaapii, Candida spp., Capnodium ramosum, Cephaloascus spp., Cephalosporium gramineum, Ceratocystis paradoxa, Chaetomium spp., Hymenoscyphus pseudoalbidus, Coccidioides spp., Cylindrosporium padi, Diplocarpon malae, Drepanopeziza campestris, Elsinoe ampelina, Epicoccum nigrum, Epidermophyton spp., Eutypa lata, Geotrichum candidum, Gibellina cerealis, Gloeocercospora sorghi, Gloeodes pomigena, Gloeosporium perennans; Gloeotinia temulenta, Griphospaeria corticola, Kabatiella lini, Leptographium microsporum, Leptosphaerulinia crassiasca, Lophodermium seditiosum, Marssonina graminicola, Microdochium nivale, Monilinia fructicola, Monographella albescens, Monosporascus cannonballus, Naemacyclus spp., Ophiostoma novo-ulmi, Paracoccidioides brasiliensis, Penicillium expansum, Pestalotia rhododendri, Petriellidium spp., Pezicula spp., Phialophora gregata, Phyllachora pomigena, Phymatotrichum omnivora, Physalospora abdita, Plectosporium tabacinum, Polyscytalum pustulans, Pseudopeziza medicaginis, Pyrenopeziza brassicae, Ramulispora sorghi, Rhabdocline pseudotsugae, Rhynchosporium secalis, Sacrocladium oryzae, Scedosporium spp., Schizothyrium pomi, Sclerotinia sclerotiorum, Sclerotinia minor; Sclerotium spp., Typhula ishikariensis, Seimatosporium mariae, Lepteutypa cupressi, Septocyta ruborum, Sphaceloma perseae, Sporonema phacidioides, Stigmina palmivora, Tapesia yallundae, Taphrina bullata, Thielviopsis basicola, Trichoseptoria fructigena, Zygophiala jamaicensis; powdery mildew diseases for example those caused by Erysiphales such as Blumeria graminis, Erysiphe polygoni, Uncinula necator, Sphaerotheca fuligena, Podosphaera leucotricha, Podospaera macularis Golovinomyces cichoracearum, Leveillula taurica, Microsphaera diffusa, Oidiopsis gossypii, Phyllactinia guttata and Oidium arachidis; molds for example those caused by Botryosphaeriales such as Dothiorella aromatica, Diplodia seriata, Guignardia bidwellii, Botrytis cinerea, Botryotinia allii, Botryotinia fabae, Fusicoccum amygdali, Lasiodiplodia theobromae, Macrophoma theicola, Macrophomina phaseolina, Phyllosticta cucurbitacearum; anthracnoses for example those caused by Glommerelales such as Colletotrichum gloeosporioides, Colletotrichum lagenarium, Colletotrichum gossypii, Glomerella cingulata, and Colletotrichum graminicola; and wilts or blights for example those caused by Hypocreales such as Acremonium strictum, Claviceps purpurea, Fusarium culmorum, Fusarium graminearum, Fusarium virguliforme, Fusarium oxysporum, Fusarium subglutinans, Fusarium oxysporum f.sp. cubense, Gerlachia nivale, Gibberella fujikuroi, Gibberella zeae, Gliocladium spp., Myrothecium verrucaria, Nectria ramulariae, Trichoderma viride, Trichothecium roseum, and Verticillium theobromae.

Basidiomycetes, including smuts for example those caused by Ustilaginales such as Ustilaginoidea virens, Ustilago nuda, Ustilago tritici, Ustilago zeae, rusts for example those caused by Pucciniales such as Cerotelium fici, Chrysomyxa arctostaphyli, Coleosporium ipomoeae, Hemileia vastatrix, Puccinia arachidis, Puccinia cacabata, Puccinia graminis, Puccinia recondita, Puccinia sorghi, Puccinia hordei, Puccinia striiformis f.sp. Hordei, Puccinia striiformis f.sp. Secalis, Pucciniastrum coryli, or Uredinales such as Cronartium ribicola, Gymnosporangium juniperi-viginianae, Melampsora medusae, Phakopsora pachyrhizi, Phragmidium mucronatum, Physopella ampelosidis, Tranzschelia discolor and Uromyces viciae-fabae; and other rots and diseases such as those caused by Cryptococcus spp., Exobasidium vexans, Marasmiellus inoderma, Mycena spp., Sphacelotheca reiliana, Typhula ishikariensis, Urocystis agropyri, Itersonilia perplexans, Corticium invisum, Laetisaria fuciformis, Waitea circinata, Rhizoctonia solani, Thanetephorus cucurmeris, Entyloma dahliae, Entylomella microspora, Neovossia moliniae and Tilletia caries.

Blastocladiomycetes, such as Physoderma maydis.

Mucoromycetes, such as Choanephora cucurbitarum.; Mucor spp.; Rhizopus arrhizus.

As well as diseases caused by other species and genera closely related to those listed above.

In addition to their fungicidal activity, the compounds and compositions comprising compounds of formula (I) as defined in the present invention may also have activity against bacteria such as Erwinia amylovora, Erwinia caratovora, Xanthomonas campestris, Pseudomonas syringae, Strptomyces scabies and other related species as well as certain protozoa.

Within the scope of the present invention, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

The useful plants and/or target crops in accordance with the invention include conventional as well as genetically enhanced or engineered varieties such as, for example, insect resistant (e.g. Bt. and VIP varieties) as well as disease resistant, herbicide tolerant (e.g. glyphosate- and glufosinate-resistant maize varieties commercially available under the trade names RoundupReady® and LibertyLink®) and nematode tolerant varieties. By way of example, suitable genetically enhanced or engineered crop varieties include the Stoneville 5599BR cotton and Stoneville 4892BR cotton varieties.

The term “useful plants” and/or “target crops” is to be understood as including also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” and/or “target crops” is to be understood as including those which naturally are or have been rendered resistant to harmful insects. This includes plants transformed by the use of recombinant DNA techniques, for example, to be capable of synthesising one or more selectively acting toxins, such as are known, for example, from toxin-producing bacteria. Examples of toxins which can be expressed include δ-endotoxins, vegetative insecticidal proteins (Vip), insecticidal proteins of bacteria colonising nematodes, and toxins produced by scorpions, arachnids, wasps and fungi. An example of a crop that has been modified to express the Bacillus thuringiensis toxin is the Bt maize KnockOut® (Syngenta Seeds). An example of a crop comprising more than one gene that codes for insecticidal resistance and thus expresses more than one toxin is VipCot® (Syngenta Seeds). Crops or seed material thereof can also be resistant to multiple types of pests (so-called stacked transgenic events when created by genetic modification). For example, a plant can have the ability to express an insecticidal protein while at the same time being herbicide tolerant, for example Herculex I® (Dow AgroSciences, Pioneer Hi-Bred International).

The term “useful plants” and/or “target crops” is to be understood as including also useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

Toxins that can be expressed by transgenic plants include, for example, insecticidal proteins from Bacillus cereus or Bacillus popilliae; or insecticidal proteins from Bacillus thuringiensis, such as δ-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

Further, in the context of the present invention there are to be understood by δ-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO03/018810).

More examples of such toxins or transgenic plants capable of synthesising such toxins are disclosed, for example, in EP-A-0 374 753, WO93/07278, WO95134656, EP-A-0 427 529, EP-A-451 878 and WO03/052073.

The processes for the preparation of such transgenic plants are generally known to the person skilled in the art and are described, for example, in the publications mentioned above. Cryl-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex 1® (maize variety that expresses a Cry1 Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.

2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.

3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.

4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.

5. IPC 531 Cotton from Monsanto Europe S.A. 270-272 Avenue de Tervuren, B-1150 Brussels, Belgium, registration number C/ES/96/02.

6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.

7. NK603 x MON 810 Maize from Monsanto Europe S. A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603 x MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

Pesticidal agents referred to herein using their common name are known, for example, from “The Pesticide Manual”, 19th Ed., British Crop Protection Council 2021.

The compounds of formula (I) as defined in the present invention may be used in unmodified form or, preferably, together with the adjuvants conventionally employed in the art of formulation. To this end they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and/or adjuvants, e.g. for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from 0.5% to 95% of the concentrate.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain from 5% to 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from 0.5% to 95% of the concentrate.

Granular formulations include both extrudates and relatively coarse particles and are usually applied without dilution to the area in which treatment is required. Typical carriers for granular formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain 5% to 25% of active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically 1 to 50 microns in diameter. The enclosed liquid typically constitutes 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely-divided form as a result of vaporisation of a low boiling dispersant solvent carrier, may also be used.

Suitable agricultural adjuvants and/or carriers that are useful in formulating the compositions of the invention in the formulation types described above are well known to those skilled in the art.

Liquid carriers that can be employed include, for example, water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc., ethylene glycol, propylene glycol, glycerine and N-methyl-2-pyrrolidinone. Water is generally the carrier of choice for the dilution of concentrates.

Suitable solid carriers include, for example, talc, titanium dioxide, pyrophyllite clay, silica, attapulgite clay, kieselguhr, chalk, diatomaxeous earth, lime, calcium carbonate, bentonite clay, fuller's earth, cotton seed hulls, wheat flour, soybean flour, pumice, wood flour, walnut shell flour and lignin.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.sub. 18 ethoxylate; alcohol-alkylene oxide addition products, such as tridecyl alcohol-C.sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants and sticking agents.

In addition, further, other biocidally active ingredients or compositions may be combined with the compositions of the invention and used in the methods of the invention and applied simultaneously or sequentially with the compositions of the invention. When applied simultaneously, these further active ingredients may be formulated together with the compositions of the invention or mixed in, for example, the spray tank. These further biocidally active ingredients may be fungicides, herbicides, insecticides, bactericides, acaricides, nematicides, plant growth regulators, and/or biologicals.

The following combinations of a compound of formula I with another active substance in a weight ratio of 1:1 are preferred (where the abbreviation “TX” means “one compound selected from the compounds defined in the Tables 1.1 to 1.45 and Table A):
(7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, (E)-6-methylhept-2-en-4-ol+TX, (E)-dec-5-en-1-yl acetate with (E)-dec-5-en-1-ol+TX, (E)-tridec-4-en-1-yl acetate+TX, (E,Z)-tetradeca-4,10-dien-1-yl acetate+TX, (Z)-dodec-7-en-1-yl acetate+TX, (Z)-hexadec-11-en-1-yl acetate+TX, (Z)-hexadec-11-enal+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, 1,2-dibromo-3-chloropropane+TX, 1,2-dichloropropane+TX, 1,2-dichloropropane with 1,3-dichloropropene+TX, 1,3-dichloropropene+TX, 14-methyloctadec-1-ene+TX, 1-hydroxy-1H-pyridine-2-thione+TX, 2-(octylthio)ethanol+TX, 2-chlorophenyl N-methylcarbamate (CPMC)+TX, 3-(4-chlorophenyl)-5-methylrhodanine+TX, 3,4-dichlorotetrahydrothiophene 1,1-dioxide+TX, 4-(quinoxalin-2-ylamino)benzenesulfonamide+TX, 4-methylnonan-5-ol with 4-methylnonan-5-one+TX, 5-methyl-6-thioxo-1,3,5-thiadiazinan-3-ylacetic acid+TX, 6-isopentenylaminopurine+TX, 8-hydroxyquinoline sulfate+TX, abamectin+TX, acequinocyl+TX, acetamiprid+TX, acetoprole+TX, acrinathrin+TX, acynonapyr+TX, Adoxophyes orana GV+TX, afidopyropen+TX, afoxolaner+TX, Agrobacterium radiobacter+TX, AKD-3088+TX, alanycarb+TX, aldicarb+TX, aldoxycarb+TX, allethrin+TX, alpha-cypermethrin+TX, alphamethrin+TX, alpha-multistriatin+TX, Amblyseius spp.+TX, amidoflumet+TX, amino acids+TX, aminocarb+TX, Anagrapha falcifera NPV+TX, Anagrus atomus+TX, Aphelinus abdominalis+TX, Aphidius colemani+TX, Aphidoletes aphidimyza+TX, apholate+TX, Autographa californica NPV+TX, AZ 60541+TX, azadirachtin+TX, azocyclotin+TX, Bacillus aizawai+TX, Bacillus chitinosporus AQ746 (NRRL Accession No B-21 618)+TX, Bacillus firmus+TX, Bacillus kurstaki+TX, Bacillus mycoides AQ726 (NRRL Accession No. B-21664)+TX, Bacillus pumilus (NRRL Accession No B-30087)+TX, Bacillus pumilus AQ717 (NRRL Accession No. B-21662)+TX, Bacillus sp. AQ175 (ATCC Accession No. 55608)+TX, Bacillus sp. AQ177 (ATCC Accession No. 55609)+TX, Bacillus sp. AQ178 (ATCC Accession No. 53522)+TX, Bacillus sphaericus Neide+TX, Bacillus subtilis AQ153 (ATCC Accession No. 55614)+TX, Bacillus subtilis AQ30002 (NRRL Accession No. B-50421)+TX, Bacillus subtilis AQ30004 (NRRL Accession No. B-50455)+TX, Bacillus subtilis AQ713 (NRRL Accession No. B-21661)+TX, Bacillus subtilis AQ743 (NRRL Accession No. B-21665)+TX, Bacillus subtilis unspecified+TX, Bacillus thuringiensis AQ52 (NRRL Accession No. B-21619)+TX, Bacillus thuringiensis BD #32 (NRRL Accession No B-21530)+TX, Bacillus thuringiensis Berliner+TX, Bacillus thuringiensis subsp. Aizawai+TX, Bacillus thuringiensis subsp. Israelensis+TX, Bacillus thuringiensis subsp. Japonensis+TX, Bacillus thuringiensis subsp. Kurstaki+TX, Bacillus thuringiensis subsp. Tenebrionis+TX, Bacillus thuringiensis subspec. kurstaki BMP 123+TX, Beauveria bassiana+TX, Beauveria brongniartii+TX, benclothiaz+TX, benomyl+TX, bensultap+TX, benzoximate+TX, benzpyrimoxan+TX, betacyfluthrin+TX, beta-cypermethrin+TX, bethoxazin+TX, bifenazate+TX, bifenthrin+TX, binapacryl+TX, bioallethrin+TX, bioresmethrin+TX, bis(tributyltin) oxide+TX, bisazir+TX, bistrifluron+TX, bisulflufen+TX, brevicomin+TX, broflanilide+TX, brofluthrinate+TX, bromoacetamide+TX, bromophos-ethyl+TX, bronopol+TX, busulfan+TX, butocarboxim+TX, butopyronoxyl+TX, butoxy(polypropylene glycol)+TX, butylpyridaben+TX, cadusafos+TX, calcium arsenate+TX, carbaryl+TX, carbofuran+TX, carbon disulfide+TX, carbosulfan+TX, cartap+TX, CAS number: 1594624-87-9+TX, CAS number: 1922957-47-8+TX, CAS number: 1255091-74-7+TX, CAS number: 1365070-72-9+TX, CAS number: 1445683-71-5+TX, CAS number: 1445684-82-1+TX, CAS number: 1594626-19-3+TX, CAS number: 1594637-65-6+TX, CAS number: 1632218-00-8+TX, CAS number: 1808115-49-2+TX, CAS number: 1922957-46-7+TX, CAS number: 1922957-48-9+TX, CAS number: 1956329-03-5+TX, CAS number: 1990457-52-7+TX, CAS number: 1990457-55-0+TX, CAS number: 1990457-57-2+TX, CAS number: 1990457-66-3+TX, CAS number: 1990457-77-6+TX, CAS number: 1990457-85-6+TX, CAS number: 2032403-97-5+TX, CAS number: 2044701-44-0+TX, CAS number: 2095470-94-1+TX, CAS Number: 2128706-04-5+TX, CAS number: 2128706-05-6+TX, CAS number: 2133042-31-4+TX, CAS number: 2133042-44-9+TX, CAS number: 2171099-09-3+TX, CAS number: 2220132-55-6+TX, CAS number: 2396747-83-2+TX, CAS number: 2408220-91-5+TX, CAS number: 2408220-94-8+TX, CAS number: 2415706-16-8+TX, Piperflanilide (CAS number: 2615135-05-0)+TX, CAS number: 2719848-60-7+TX, CAS number: RNA (Leptinotarsa decemlineata-specific recombinant double-stranded interfering GS2)+TX, chlorantraniliprole+TX, chlordane+TX, chlorfenapyr+TX, chloropicrin+TX, chloroprallethrin+TX, chlorpyrifos+TX, chromafenozide+TX, Chrysoperla carnea+TX, clenpirin+TX, cloethocarb+TX, clothianidin+TX, codlelure+TX, codlemone+TX, copper acetoarsenite+TX, copper dioctanoate+TX, copper hydroxide+TX, copper sulfate+TX, cresol+TX, crufomate+TX, Cryptolaemus montrouzieri+TX, cuelure+TX, cyanofenphos+TX, cyantraniliprole+TX, cybutryne+TX, cyclaniliprole+TX, cyclobutrifluram+TX, cycloprothrin+TX, cycloxaprid+TX, Cydia pomonella GV+TX, cyenopyrafen+TX, cyetpyrafen+TX, cyflumetofen+TX, cyfluthrin+TX, cyhalodiamide+TX, cylohalothrin+TX, cypermethrin+TX, cyphenothrin+TX, cyproflanilide+TX, cyromazine+TX, cytokinins+TX, Dacnusa sibirica+TX, dazomet+TX, DBCP+TX, DCIP+TX, deltamethrin+TX, diafenthiuron+TX, dialifos+TX, diamidafos+TX, dibrom+TX, dibutyl adipate+TX, dibutyl phthalate+TX, dibutyl succinate+TX, dichlofenthion+TX, dichlone+TX, dichlorophen+TX, dicliphos+TX, dicloromezotiaz+TX, diethyltoluamide+TX, diflubenzuron+TX, Diglyphus isaea+TX, dimatif+TX, dimethoate+TX, dimethyl carbate+TX, dimethyl phthalate+TX, dimpropyridaz+TX, dinactin+TX, dinocap+TX, dinotefuran+TX, dioxabenzofos+TX, dipyrithione+TX, disparlure+TX, D-limonene+TX, dodec-8-en-1-yl acetate+TX, dodec-9-en-1-yl acetate+TX, dodeca-8,10-dien-1-yl acetate+TX, dodicin+TX, dominicalure+TX, doramectin+TX, emamectin+TX, emamectin benzoate+TX, empenthrin+TX, Encarsia formosa+TX, endothal+TX, endrin+TX, eprinomectin+TX, epsilon-momfluorothrin+TX, epsilon-metofluthrin+TX, Eretmocerus eremicus+TX, esfenvalerate+TX, ethion+TX, ethiprole+TX, ethoprophos+TX, ethyl 4-methyloctanoate+TX, ethyl hexanediol+TX, ethylene dibromide+TX, etofenprox+TX, etoxazole+TX, etpyrafen+TX, eugenol+TX, Extract of seaweed and fermentation product derived from melasse+TX, Extract of seaweed and fermentation product derived from melasse comprising urea+TX, Extract of seaweed and fermented plant products+TX, Extract of seaweed and fermented plant products comprising phytohormones, vitamins, EDTA-chelated copper, zinc, and iron+TX, famphur+TX, fenaminosulf+TX, fenamiphos+TX, fenazaquin+TX, fenfluthrin+TX, fenitrothion+TX, fenmezoditiaz+TX, fenobucarb+TX, fenothiocarb+TX, fenoxycarb+TX, fenpropathrin+TX, fenpyrad+TX, fenpyroximate+TX, fensulfothion+TX, fenthion+TX, fentin+TX, fentinacetate+TX, fenvalerate+TX, ferric phosphate+TX, fipronil+TX, flometoquin+TX, flonicamid+TX, fluacrypyrim+TX, fluazaindolizine+TX, fluazuron+TX, flubendiamide+TX, flubenzimine+TX, fluchlordiniliprole+TX, flucitrinate+TX, flucycloxuron+TX, flucythrinate+TX, fluensulfone [318290-98-1]+TX, fluensulfone+TX, flufenerim+TX, flufenprox+TX, flufiprole+TX, fluhexafon+TX, flumethrin+TX, fluopyram+TX, flupyradifurone+TX, flupyrimin+TX, flupyroxystrobin+TX, fluralaner+TX, fluvalinate+TX, fluxametamide+TX, formaldehyde+TX, fosthiazate+TX, fosthietan+TX, frontalin+TX, furfural+TX, gamma-cyhalothrin+TX, Gossyplure® (1:1 mixture of the (Z,E) and (Z,Z) isomers of hexadeca-7,11-dien-1-yl-acetate)+TX, grandlure+TX, grandlure I+TX, grandlure II+TX, grandlure III+TX, grandlure IV+TX, Granulovirus+TX, guadipyr+TX, GY-81+TX, halfenprox+TX, halofenozide+TX, Harpin+TX, Helicoverpa armigera Nucleopolyhedrovirus+TX, Helicoverpa zea NPV+TX, Helicoverpa zea Nucleopolyhedrovirus+TX, Heliothis punctigera Nucleopolyhedrovirus+TX, Heliothis virescens Nucleopolyhedrovirus+TX, hemel+TX, hempa+TX, heptafluthrin+TX, heterophos+TX, Heterorhabditis bacteriophora and H. megidis+TX, hexalure+TX, hexamide+TX, hexythiazox+TX, Hippodamia convergens+TX, hydramethylnon+TX, hydrargaphen+TX, hydrated lime+TX, imicyafos+TX, imidacloprid+TX, imiprothrin+TX, Indazapyroxamet+TX, indoxacarb+TX, iodomethane+TX, iprodione+TX, ipsdienol+TX, ipsenol+TX, isamidofos+TX, isazofos+TX, isocycloseram+TX, Isoflualanam (CAS number: 2892524-05-7)+TX, isothioate+TX, ivermectin+TX, japonilure+TX, kappa-bifenthrin+TX, kappa-tefluthrin+TX, kasugamycin+TX, kasugamycin hydrochloride hydrate+TX, kinetin+TX, lambda-cyhalothrin+TX, ledprona+TX, lepimectin+TX, Leptomastix dactylopii+TX, lineatin+TX, litlure+TX, looplure+TX, lotilaner+TX, lufenuron+TX, Macrolophus caliginosus+TX, Mamestra brassicae NPV+TX, mecarphon+TX, medlure+TX, megatomoic acid+TX, metaflumizone+TX, metaldehyde+TX, metam+TX, metam-potassium+TX, metam-sodium+TX, Metaphycus helvolus+TX, Metarhizium anisopliae var. acridum+TX, Metarhizium anisopliae var. anisopliae+TX, Metarhizium spp.+TX, metepa+TX, methiocarb+TX, methiotepa+TX, methomyl+TX, methoquin-butyl+TX, methoxyfenozide+TX, methyl apholate+TX, methyl bromide+TX, methyl eugenol+TX, methyl isothiocyanate+TX, methylneodecanamide+TX, metofluthrin+TX, metolcarb+TX, mexacarbate+TX, milbemectin+TX, milbemycin oxime+TX, momfluorothrin+TX, morzid+TX, moxidectin+TX, muscalure+TX, Muscodor albus 620 (NRRL Accession No. 30547)+TX, Muscodor roseus A3-5 (NRRL Accession No. 30548)+TX, Myrothecium verrucaria composition+TX, nabam+TX, NC-184+TX, Neem tree based products+TX, Neodiprion sertifer NPV and N. lecontei NPV+TX, nickel bis(dimethyldithiocarbamate)+TX, niclosamide+TX, niclosamide-olamine+TX, nicofluprole+TX, nitenpyram+TX, nithiazine+TX, nitrapyrin+TX, octadeca-2,13-dien-1-yl acetate+TX, octadeca-3,13-dien-1-yl acetate+TX, octhilinone+TX, omethoate+TX, orfralure+TX, Orius spp.+TX, oryctalure+TX, ostramone+TX, oxamate+TX, oxamyl+TX, oxazosulfyl+TX, oxolinic acid+TX, oxytetracycline+TX, Paecilomyces fumosoroseus+TX, Paecilomyces lilacinus+TX, parathion-ethyl+TX, Pasteuria nishizawae+TX, Pasteuria penetrans+TX, Pasteuria ramosa+TX, Pasteuria thornei+TX, Pasteuria usgae+TX, P-cymene+TX, penfluron+TX, pentachlorophenol+TX, permethrin+TX, phenothrin+TX, phorate+TX, phosphamidon+TX, phosphocarb+TX, Phytoseiulus persimilis+TX, picaridin+TX, pioxaniliprole+TX, piperazine+TX, piperonylbutoxide+TX, pirimicarb+TX, pirimiphos-ethyl+TX, pirimiphos-methyl+TX, Plutella xylostella Granulosis virus+TX, Plutella xylostella Nucleopolyhedrovirus+TX, Polyhedrosis virus+TX, potassium and molybdenum and EDTA-chelated manganese+TX, potassium ethylxanthate+TX, potassium hydroxyquinoline sulfate+TX, prallethrin+TX, probenazole+TX, profenofos+TX, profluthrin+TX, propargite+TX, propetamphos+TX, propoxur+TX, prothiophos+TX, protrifenbute+TX, pyflubumide+TX, pymetrozine+TX, pyraclofos+TX, pyrafluprole+TX, pyrethrum+TX, pyridaben+TX, pyridalyl+TX, pyridin-4-amine+TX, pyrifluquinazon+TX, pyrimidifen+TX, pyriminostrobin+TX, pyriprole [394730-71-3]+TX, pyriprole+TX, pyriproxyfen+TX, QRD 420 (a terpenoid blend)+TX, QRD 452 (a terpenoid blend)+TX, QRD 460 (a terpenoid blend)+TX, Quillaja saponaria+TX, quinoclamine+TX, quinonamid+TX, resmethrin+TX, Rhodococcus globerulus AQ719 (NRRL Accession No B-21663)+TX, sarolaner+TX, S-bioallethrin+TX, sebufos+TX, selamectin+TX, siglure+TX, silafluofen+TX, simazine+TX, sodium pentachlorophenoxide+TX, sordidin+TX, spidoxamat+TX, spinetoram+TX, spinosad+TX, spirobudifen+TX, spirodiclofen+TX, spiromesifen+TX, spiropidion+TX, spirotetramat+TX, Spodoptera exigua multicapsid nuclear polyhedrosis virus+TX, Spodoptera frugiperda Nucleopolyhedrovirus+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Streptomyces galbus (NRRL Accession No. 30232)+TX, Streptomyces sp. (NRRL Accession No. B-30145)+TX, streptomycin+TX, streptomycin sesquisulfate+TX, strychnine+TX, sulcatol+TX, sulfiflumin (CAS number: 2377084-09-6)+TX, sulfoxaflor+TX, tazimcarb+TX, tebufenozide+TX, tebufenpyrad+TX, tebupirimiphos+TX, tecloftalam+TX, tefluthrin+TX, temephos+TX, tepa+TX, terbam+TX, terbufos+TX, terpenoid blend+TX, tetrachlorantraniliprole+TX, tetrachlorothiophene+TX, tetradec-11-en-1-yl acetate+TX, tetradiphon+TX, tetramethrin+TX, tetramethylfluthrin+TX, tetranactin+TX, tetraniliprole+TX, theta-cypermethrin+TX, thiacloprid+TX, thiafenox+TX, thiamethoxam+TX, thiocyclam+TX, thiodicarb+TX, thiofanox+TX, thiohempa+TX, thiomersal+TX, thiometon+TX, thionazin+TX, thiophanate+TX, thiosultap+TX, thiotepa+TX, tigolaner+TX, tiorantraniliprole+TX, tioxazafen+TX, tolfenpyrad+TX, toxaphene+TX, tralomethrin+TX, transfluthrin+TX, tretamine+TX, triazamate+TX, triazophos+TX, triazuron+TX, tributyltin oxide+TX, trichlorfon+TX, trichloronate+TX, trichlorphon+TX, Trichogramma spp.+TX, trifenmorph+TX, trifluenfuronate+TX, triflumezopyrim+TX, trimedlure+TX, trimedlure A+TX, trimedlure B1+TX, trimedlure B2+TX, trimedlure C+TX, trimethacarb+TX, triphenyltin acetate+TX, triphenyltin hydroxide+TX, trunc-call+TX, tyclopyrazoflor+TX, Typhlodromus occidentalis+TX, uredepa+TX, Verticillium lecanii+TX, Verticillium spp.+TX, xylenols+TX, YI-5302+TX, zeatin+TX, zeta-Cypermethrin+TX; N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-ethyl-N′-[5-methoxy-2-methyl-4-[(2-trifuoromethyl)tetrahydrofuran-2-yl]phenyl]-N-methyl-formamidine (these compounds may be prepared from the methods described in WO2019/110427)+TX, (3′,4′,5′-trifluoro-biphenyl-2-yl)-amide+TX, (3-methylisoxazol-5-yl)-[4-[5-(trifluoromethyl)-1,2,4-oxadiazol-3-yl]phenyl]methanone (these compounds may be prepared from the methods described in WO 2017/220485)+TX, (4-phenoxyphenyl)methyl 2-amino-6-methyl-pyridine-3-carboxylate (this compound may be prepared from the methods described in WO 2014/006945)+TX, (5-methyl-2-pyridyl)-[4-[5-(trifluoromethyl)-1,2,4-oxadiazol-3-yl]phenyl]methanone+TX, (7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, (E)-6-methylhept-2-en-4-ol+TX, (E)-dec-5-en-1-yl acetate with (E)-dec-5-en-1-ol+TX, (E)-tridec-4-en-1-yl acetate+TX, (E,Z)-tetradeca-4,10-dien-1-yl acetate, +TX, (R)-3-(difluoromethyl)-1-methyl-N-[1,1,3-trimethylindan-4-yl]pyrazole-4-carboxamide+TX, (Z)-dodec-7-en-1-yl acetate+TX, (Z)-hexadec-11-en-1-yl acetate+TX, (Z)-hexadec-11-enal+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (Z,2E)-5-[1-(2,4-dichlorophenyl)pyrazol-3-yl]oxy-2-methoxyimino-N,3-dimethyl-pent-3-enamide (this compound may be prepared from the methods described in WO 2018/153707)+TX, (Z,2E)-5-[1-(4-chlorophenyl)pyrazol-3-yl]oxy-2-methoxyimino-N,3-dimethyl-pent-3-enamide+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chlorophenyl]methanesulfonate+TX, 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 1-(6-chloro-7-methyl-pyrazolo[1,5-a]pyridin-3-yl)-4,4-difluoro-3,3-dimethyl-isoquinoline (these compounds may be prepared from the methods described in WO2017/025510)+TX, 1,1-bis(4-chlorophenyl)-2-ethoxyethanol+TX, 1,1-dichloro-2,2-bis(4-ethylphenyl)ethane+TX, 1,2-dibromo-3-chloropropane+TX, 1,2-dichloropropane with 1,3-dichloropropene+TX, 1,3-dichloropropene+TX, 1,3-dimethoxy-1-[[4-[5-(trifluoromethyl)-1,2,4-oxadiazol-3-yl]phenyl]methyl]urea+TX, 1-[2-[[1-(4-chlorophenyl)pyrazol-3-yl]oxymethyl]-3-methyl-phenyl]-4-methyl-tetrazol-5-one+TX, 10-dien-1-yl acetate+TX, 14-methyloctadec-1-ene+TX, 1-bromo-2-chloroethane+TX, 1-dichloro-1-nitroethane+TX, 1-hydroxy-1H-pyridine-2-thione+TX, 1-methoxy-3-methyl-1-[[4-[5-(trifluoromethyl)-1,2,4-oxadiazol-3-yl]phenyl]methyl]urea+TX, 1-methyl-4-[3-methyl-2-[[2-methyl-4-(3,4,5-trimethylpyrazol-1-yl)phenoxy]methyl]phenyl]tetrazol-5-one+TX, 2-(difluoromethyl)-N-((3R)-1, 1, 3-trimethylindan-4-yl) pyridine-3-carboxamide+TX, 2-(difluoromethyl)-N-((3R)-1, 1, 3-trimethylindan-4-yl) pyridine-3-carboxamide+TX, 2-(1,3-dithiolan-2-yl)phenyl dimethylcarbamate+TX, 2-(2-butoxyethoxy)ethyl piperonylate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-(difluoromethyl)-N-(3-ethyl-1,1-dimethyl-indan-4-yl)pyridine-3-carboxamide+TX, 2-(difluoromethyl)-N-[(3R)-3-ethyl-1,1-dimethyl-indan-4-yl]pyridine-3-carboxamide+TX, 2-(difluoromethyl)-N-[(3S)-3-ethyl-1,1-dimethyl-indan-4-yl]pyridine-3-carboxamide (this compound may be prepared from the methods described in WO 2014/095675)+TX, 2-(difluoromethyl)-N-[3-ethyl-1,1-dimethyl-indan-4-yl]pyridine-3-carboxamide+TX, 2-(octylthio)ethanol+TX, 2,2,2-trichloro-1-(3,4-dichlorophenyl)ethyl acetate+TX, 2,2-dichlorovinyl 2-ethylsulfinylethyl methyl phosphate+TX, 2,2-difluoro-N-methyl-2-[4-[5-(trifluoromethyl)-1,2,4-oxadiazol-3-yl]phenyl]acetamide+TX, 2,4-dichlorophenyl benzenesulfonate+TX, 2,6-Dimethyl-1H,5H-[1,4]dithiino[2,3-c:5,6-c′]dipyrrole-1,3,5,7(2H,6H)-tetrone (this compound may be prepared from the methods described in WO 2011/138281)+TX, 2-[2-fluoro-6-[(8-fluoro-2-methyl-3-quinolyl)oxy]phenyl]propan-2-ol+TX, 2-[6-(4-bromophenoxy)-2-(trifluoromethyl)-3-pyridyl]-1-(1,2,4-triazol-1-yl)propan-2-ol (this compound may be prepared from the methods described in WO 2017/029179)+TX, 2-[6-(4-chlorophenoxy)-2-(trifluoromethyl)-3-pyridyl]-1-(1,2,4-triazol-1-yl)propan-2-ol (this compound may be prepared from the methods described in WO 2017/029179)+TX, 2-chlorovinyl diethyl phosphate+TX, 2-fluoro-N-methyl-N-1-naphthylacetamide+TX, 2-imidazolidone+TX, 2-isovalerylindan-1,3-dione+TX, 2-methyl(prop-2-ynyl)aminophenyl methylcarbamate+TX, 2-oxo-N-propyl-2-[4-[5-(trifluoromethyl)-1,2,4-oxadiazol-3-yl]phenyl]acetamide (this compound may be prepared from the methods described in WO 2018/065414)+TX, 2-thiocyanatoethyl laurate+TX, 3-(4,4-difluoro-3,3-dimethyl-1-isoquinolyl)-7,8-dihydro-6H-cyclopenta[e]benzimidazole (these compounds may be prepared from the methods described in WO2016/156085)+TX, 3-(4,4-difluoro-3,4-dihydro-3,3-dimethylisoquinolin-1-yl)quinolone+TX, 3-(4-chlorophenyl)-5-methylrhodanine+TX, 3-(difluoromethyl)-1-methyl-N-[1,1,3-trimethylindan-4-yl]pyrazole-4-carboxamide+TX, 3,4-dichlorotetrahydrothiophene 1,1-dioxide+TX, 3-[2-(1-chlorocyclopropyl)-3-(2-fluorophenyl)-2-hydroxy-propyl]imidazole-4-carbonitrile (this compound may be prepared from the methods described in WO 2016/156290)+TX, 3-[2-(1-chlorocyclopropyl)-3-(3-chloro-2-fluoro-phenyl)-2-hydroxy-propyl]imidazole-4-carbonitrile (this compound may be prepared from the methods described in WO 2016/156290)+TX, 3-bromo-1-chloroprop-1-ene+TX, 3-chloro-6-methyl-5-phenyl-4-(2,4,6-trifluorophenyl)pyridazine+TX, 3-difluoromethyl-1-methyl-1H-pyrazole-4-carboxylic acid+TX, 3-ethyl-1-methoxy-1-[[4-[5-(trifluoromethyl)-1,2,4-oxadiazol-3-yl]phenyl]methyl]urea+TX, 3-methyl-1-phenylpyrazol-5-yl dimethylcarbamate+TX, 4-(2-bromo-4-fluorophenyl)-N-(2-chloro-6-fluorophenyl)-1, 3-dimethyl-1H-pyrazol-5-amine+TX, 4-(2,6-difluorophenyl)-6-methyl-5-phenyl-pyridazine-3-carbonitrile+TX, 4-(2-bromo-4-fluoro-phenyl)-N-(2-chloro-6-fluoro-phenyl)-2,5-dimethyl-pyrazol-3-amine+TX, 4-(quinoxalin-2-ylamino)benzenesulfonamide+TX, 4,4-difluoro-1-(5-fluoro-4-methyl-benzimidazol-1-yl)-3,3-dimethyl-isoquinoline+TX, 4,4-difluoro-3,3-dimethyl-1-(6-methylpyrazolo[1,5-a]pyridin-3-yl)isoquinoline+TX, 4,4-difluoro-3,3-dimethyl-1-(7-methylpyrazolo[1,5-a]pyridin-3-yl)isoquinoline+TX, 4,4-dimethyl-2-[[4-[5-(trifluoromethyl)-1,2,4-oxadiazol-3-yl]phenyl]methyl]isoxazolidin-3-one+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, 4-chloro-2-(2-chloro-2-methyl-propyl)-5-[(6-iodo-3-pyridyl)methoxy]pyrdazin-3-one+TX, 4-chlorophenyl phenyl sulfone+TX, 4-methyl(prop-2-ynyl)amino-3,5-xylyl methylcarbamate+TX, 4-methylnonan-5-ol with 4-methylnonan-5-one+TX, 5-(1,3-benzodioxol-5-yl)-3-hexylcyclohex-2-enone+TX, 5,5-dimethyl-2-[[4-[5-(trifluoromethyl)-1,2,4-oxadiazol-3-yl]phenyl]methyl]isoxazolidin-3-one+TX, 5,5-dimethyl-3-oxocyclohex-1-enyl dimethylcarbamate+TX, 5-amino-1,3,4-thiadiazole-2-thiol zinc salt (2:1)+TX, 5-methyl-6-thioxo-1,3,5-thiadiazinan-3-ylacetic acid+TX, 6-chloro-3-(3-cyclopropyl-2-fluoro-phenoxy)-N-[2-(2,4-dimethylphenyl)-2,2-difluoro-ethyl]-5-methyl-pyridazine-4-carboxamide (may be prepared from the methods described in WO 2020/109391)+TX, 6-chloro-3-(3-cyclopropyl-2-fluoro-phenoxy)-N-[2-(3,4-dimethylphenyl)-2,2-difluoro-ethyl]-5-methyl-pyridazine-4-carboxamide (may be prepared from the methods described in WO 2020/109391)+TX, 6-chloro-4,4-difluoro-3,3-dimethyl-1-(4-methylbenzimidazol-1-yl)isoquinoline+TX, 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide (may be prepared from the methods described in WO 2020/109391)+TX, 6-ethyl-5,7-dioxo-pyrrolo[4,5][1,4]dithiino[1,2-c]isothiazole-3-carbonitrile+TX, 6-isopentenylaminopurine+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, 8-hydroxyquinoline sulfate+TX, acethion+TX, acetoprole+TX, acibenzolar+TX, acibenzolar-S-methyl+TX, acrylonitrile+TX, Adoxophyes orana GV+TX, Agrobacterium radiobacter+TX, aldoxycarb+TX, aldrin+TX, allosamidin+TX, allyxycarb+TX, alpha-chlorohydrin+TX, alpha-ecdysone+TX, alpha-multistriatin+TX, aluminium phosphide+TX, Amblyseius spp.+TX, amectotractin+TX, ametoctradin+TX, amidithion+TX, amidothioate+TX, aminocarb+TX, aminopyrifen+TX, amisulbrom+TX, amiton+TX, amiton hydrogen oxalate+TX, amitraz+TX, anabasine+TX, Anagrapha falcifera NPV+TX, Anagrus atomus+TX, ancymidol+TX, anilazine+TX, anisiflupurin+TX, anthraquinone+TX, antu+TX, Aphelinus abdominalis+TX, Aphidius colemani+TX, Aphidoletes aphidimyza+TX, apholate+TX, aramite+TX, arsenous oxide+TX, athidathion+TX, Autographa californica NPV+TX, azaconazole+TX, azamethiphos+TX, azobenzene+TX, azothoate+TX, azoxystrobin+TX, Bacillus sphaericus Neide+TX, Bacillus thuringiensis delta endotoxins+TX, barium carbonate+TX, barium hexafluorosilicate+TX, barium polysulfide+TX, barthrin+TX, Bayer 22/190+TX, Bayer 22408+TX, Beauveria brongniartii+TX, benalaxyl+TX, benclothiaz+TX, benomyl+TX, benoxafos+TX, benthiavalicarb+TX, benzothiostrobin+TX, benzovindiflupyr+TX, benzyl benzoate+TX, beta-cyfluthrin+TX, beta-cypermethrin+TX, bethoxazin+TX, bioethanomethrin+TX, biopermethrin+TX, bis(2-chloroethyl) ether+TX, bis(tributyltin) oxide+TX, bisazir+TX, bisthiosemi+TX, bitertanol+TX, bixafen+TX, blasticidin-S+TX, borax+TX, bordeaux mixture+TX, boscalid+TX, brevicomin+TX, brodifacoum+TX, brofenvalerate+TX, bromadiolone+TX, bromethalin+TX, bromfenvinfos+TX, bromoacetamide+TX, bromocyclen+TX, bromo-DDT+TX, bromophos+TX, bromopropylate+TX, bromuconazole+TX, bronopol+TX, bufencarb+TX, bupirimate+TX, buprofezin+TX, busulfan+TX, but-3-ynyl N-[6-[[(Z)-[(1-methyltetrazol-5-yl)-phenyl-methylene]amino]oxymethyl]-2-pyridyl]carbamate+TX, butacarb+TX, butathiofos+TX, butocarboxim+TX, butonate+TX, butopyronoxyl+TX, butoxy(polypropylene glycol)+TX, butoxycarboxim+TX, butylpyridaben+TX, calcium arsenate+TX, calcium cyanide+TX, calcium polysulfide+TX, camphechlor+TX, captafol+TX, captan+TX, carbanolate+TX, carbendazim+TX, carbon disulfide+TX, carbon tetrachloride+TX, carbophenothion+TX, carboxin+TX, cartap hydrochloride+TX, CAS Number: 2132414-04-9+TX, CAS Number: 2344721-61-3+TX, cevadine+TX, chinomethionat+TX, chloralose+TX, chlorbenside+TX, chlorbicyclen+TX, chlordane+TX, chlordecone+TX, chlordimeform+TX, chlordimeform hydrochloride+TX, chlorfenethol+TX, chlorfenson+TX, chlorfensulfide+TX, chlorobenzilate+TX, chloroform+TX, chloroinconazide+TX, chloromebuform+TX, chloromethiuron+TX, chloroneb+TX, chlorophacinone+TX, chloropicrin+TX, chloropropylate+TX, chlorothalonil+TX, chlorphoxim+TX, chlorprazophos+TX, chlorthiophos+TX, chlozolinate+TX, cholecalciferol+TX, Chrysoperla carnea+TX, cinerin I+TX, cinerin II+TX, cinerins+TX, cismethrin+TX, cis-resmethrin+TX, clocythrin+TX, closantel+TX, codlelure+TX, codlemone+TX, copper acetoarsenite+TX, copper arsenate+TX, copper dioctanoate+TX, copper hydroxide+TX, copper naphthenate+TX, copper oleate+TX, copper oxide+TX, copper oxychloride+TX, copper sulfate+TX, coumachlor+TX, coumafuryl+TX, coumaphos+TX, coumatetralyl+TX, coumethoxystrobin (jiaxiangjunzhi)+TX, coumithoate+TX, coumoxystrobin+TX, cresol+TX, crimidine+TX, crotamiton+TX, crotoxyphos+TX, crufomate+TX, cryolite+TX, Cryptolaemus montrouzieri+TX, CS 708+TX, cuelure+TX, cufraneb+TX, cyanofenphos+TX, cyanophos+TX, cyanthoate+TX, cyazofamid+TX, cybutryne+TX, cyclethrin+TX, cyclobutrifluram+TX, Cydia pomonella GV+TX, cyflufenamid+TX, cymiazole+TX, cymoxanil+TX, cyproconazole+TX, cyprodinil+TX, cythioate+TX, cytokinins+TX, Dacnusa sibirica+TX, DAEP+TX, dazomet+TX, DCIP+TX, DCPM+TX, DDT+TX, debacarb+TX, decarbofuran+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, diamidafos+TX, dibutyl adipate+TX, dibutyl phthalate+TX, dibutyl succinate+TX, dicapthon+TX, dichlobentiazox+TX, dichlofenthion+TX, dichlofluanid+TX, dichlone+TX, dichlorophen+TX, dichlorvos+TX, dichlozoline+TX, dicliphos+TX, diclocymet+TX, diclomezine+TX, dicloran+TX, dicresyl+TX, dicyclanil+TX, dicyclopentadiene+TX, dieldrin+TX, dienochlor+TX, diethofencarb+TX, diethyl 5-methylpyrazol-3-yl phosphate+TX, diethyltoluamide+TX, difenacoum+TX, difenoconazole+TX, difethialone+TX, diflovidazin+TX, Diglyphus isaea+TX, dilor+TX, dimatif+TX, dimefluthrin+TX, dimefox+TX, dimetan+TX, dimethirimol+TX, dimethomorph+TX, dimethrin+TX, dimethyl carbate+TX, dimethyl phthalate+TX, dimethylvinphos+TX, dimetilan+TX, dimoxystrobin+TX, dinex+TX, dinex-diclexine+TX, diniconazole+TX, dinocap-4+TX, dinocap-6+TX, dinocton+TX, dinopenton+TX, dinoprop+TX, dinosam+TX, dinoseb+TX, dinosulfon+TX, dinoterbon+TX, diofenolan+TX, dioxabenzofos+TX, dioxathion+TX, diphacinone+TX, diphenyl sulfone+TX, dipymetitrone+TX, dipyrithione+TX, disparlure+TX, disulfiram+TX, dithianon+TX, dithicrofos+TX, DNOC+TX, dodec-8-en-1-yl acetate+TX, dodec-9-en-1-yl acetate+TX, dodeca-8+TX, dodemorph+TX, dodicin+TX, dodine+TX, dofenapyn+TX, dominicalure+TX, doramectin+TX, DSP+TX, d-tetramethrin+TX, ecdysterone+TX, edifenphos+TX, EI 1642+TX, EMPC+TX, Encarsia formosa+TX, endothal+TX, endothion+TX, enestroburin+TX, enoxastrobin+TX, EPBP+TX, epoxiconazole+TX, eprinomectin+TX, Eretmocerus eremicus+TX, ergocalciferol+TX, etaphos+TX, ethaboxam+TX, ethiofencarb+TX, ethirimol+TX, ethoate-methyl+TX, ethyl 1-[[4-[(Z)-2-ethoxy-3,3,3-trifluoro-prop-1-enoxy]phenyl]methyl]pyrazole-3-carboxylate (may be prepared from the methods described in WO 2020/056090)+TX, ethyl 1-[[4-[[2-(trifluoromethyl)-1,3-dioxolan-2-yl]methoxy]phenyl]methyl]pyrazole-3-carboxylate (may be prepared from the methods described in WO 2020/056090)+TX, ethyl 1-[[4-[5-(trifluoromethyl)-1,2,4-oxadiazol-3-yl]phenyl]methyl]pyrazole-4-carboxylate+TX, ethyl 1-[[5-[5-(trifluoromethyl)-1,2,4-oxadiazol-3-yl]-2-thienyl]methyl]pyrazole-4-carboxylate (this compound may be prepared from the methods described in WO 2018/158365)+TX, ethyl 4-methyloctanoate+TX, ethyl formate+TX, ethyl hexanediol+TX, ethylene dibromide+TX, ethylene dichloride+TX, ethylene oxide+TX, etridiazole+TX, etrimfos+TX, eugenol+TX, EXD+TX, famoxadone+TX, farnesol+TX, farnesol with nerolidol+TX, fenamidone+TX, fenaminosulf+TX, fenaminstrobin+TX, fenarimol+TX, fenazaflor+TX, fenbuconazole+TX, fenbutatin oxide+TX, fenchlorphos+TX, fenethacarb+TX, fenfuram+TX, fenhexamid+TX, fenitrothion+TX, fenothiocarb+TX, fenoxacrim+TX, fenoxanil+TX, fenpiclonil+TX, fenpicoxamid+TX, fenpirithrin+TX, fenpropidin+TX, fenpropimorph+TX, fenpyrad+TX, fenpyrazamine+TX, fenpyroximate+TX, fenson+TX, fensulfothion+TX, fenthion+TX, fenthion-ethyl+TX, fentin+TX, fentrifanil+TX, ferbam+TX, ferimzone+TX, ferric phosphate+TX, flocoumafen+TX, florylpicoxamid+TX, fluazinam+TX, flubeneteram+TX, flubenzimine+TX, flucofuron+TX, flucycloxuron+TX, fludioxonil+TX, fluenetil+TX, flufenoxadiazam+TX, flufenoxystrobin+TX, fluindapyr+TX, flumetylsulforim+TX, flumorph+TX, fluopicolide+TX, fluopimomide+TX, fluopyram+TX, fluorbenside+TX, fluoroacetamide+TX, fluoroimide+TX, fluoxapiprolin+TX, fluoxastrobin+TX, fluoxytioconazole+TX, flupropadine+TX, flupropadine hydrochloride+TX, fluquinconazole+TX, flusilazole+TX, flusulfamide+TX, flutianil+TX, flutolanil+TX, flutriafol+TX, fluxapyroxad+TX, FMC 1137+TX, folpet+TX, formaldehyde+TX, formetanate+TX, formetanate hydrochloride+TX, formparanate+TX, fosetyl-aluminium+TX, fosmethilan+TX, fospirate+TX, fosthietan+TX, frontalin+TX, fuberidazole+TX, furalaxyl+TX, furametpyr+TX, furathiocarb+TX, furethrin+TX, furfural+TX, gamma-HCH+TX, glyodin+TX, grandlure+TX, grandlure I+TX, grandlure II+TX, grandlure III+TX, grandlure IV+TX, guazatine+TX, guazatine acetates+TX, halfenprox+TX, HCH+TX, hemel+TX, hempa+TX, HEOD+TX, heptachlor+TX, heterophos+TX, Heterorhabditis bacteriophora and H. megidis+TX, hexaconazole+TX, hexadecyl cyclopropanecarboxylate+TX, hexalure+TX, hexamide+TX, HHDN+TX, Hippodamia convergens+TX, hydrargaphen+TX, hydrated lime+TX, hydrogen cyanide+TX, hymexazol+TX, hyquincarb+TX, imanin+TX, imazalil+TX, imibenconazole+TX, iminoctadine+TX, inpyrfluxam+TX, ipconazole+TX, ipfentrifluconazole+TX, ipflufenoquin+TX, iprobenphos+TX, iprodione+TX, iprovalicarb+TX, ipsdienol+TX, ipsenol+TX, IPSP+TX, isamidofos+TX, isazofos+TX, isobenzan+TX, isocarbophos+TX, isodrin+TX, isofenphos+TX, isofetamid+TX, isoflucypram+TX, isolane+TX, isoprothiolane+TX, isopyrazam+TX, isotianil+TX, isoxathion+TX, japonilure+TX, jasmolin I+TX, jasmolin II+TX, jodfenphos+TX, juvenile hormone I+TX, juvenile hormone II+TX, juvenile hormone Ill+TX, kadethrin+TX, kasugamycin+TX, kasugamycin hydrochloride hydrate+TX, kelevan+TX, kinetin+TX, kinoprene+TX, kresoxim-methyl+TX, lead arsenate+TX, Leptomastix dactylopii+TX, leptophos+TX, lindane+TX, lineatin+TX, lirimfos+TX, litlure+TX, looplure+TX, Ivbenmixianan+TX, lythidathion+TX, Macrolophus caliginosus+TX, magnesium phosphide+TX, malonoben+TX, Mamestra brassicae NPV+TX, mancopper+TX, mancozeb+TX, mandestrobin+TX, mandipropamid+TX, maneb+TX, mazidox+TX, m-cumenyl methylcarbamate+TX, mecarbam+TX, mecarphon+TX, medlure+TX, mefentrifluconazole+TX, megatomoic acid+TX, menazon+TX, mepanipyrim+TX, meperfluthrin+TX, mephosfolan+TX, mepronil+TX, mercuric oxide+TX, mercurous chloride+TX, mesulfen+TX, mesulfenfos+TX, metalaxyl+TX, metam+TX, metam-potassium+TX, metam-sodium+TX, Metaphycus helvolus+TX, Metarhizium anisopliae var. acridum+TX, Metarhizium anisopliae var. anisopliae+TX, metarylpicoxamid+TX, metconazole+TX, metepa+TX, methacrifos+TX, methanesulfonyl fluoride+TX, methasulfocarb+TX, methiotepa+TX, methocrotophos+TX, methoprene+TX, methoquin-butyl+TX, methothrin+TX, methoxychlor+TX, methyl (Z)-2-(5-cyclohexyl-2-methyl-phenoxy)-3-methoxy-prop-2-enoate+TX, methyl (Z)-2-(5-cyclopentyl-2-methyl-phenoxy)-3-methoxy-prop-2-enoate (these compounds may be prepared from the methods described in WO2020/193387)+TX, methyl (Z)-2-[5-(3-isopropylpyrazol-1-yl)-2-methyl-phenoxy]-3-methoxy-prop-2-enoate+TX, methyl (Z)-3-methoxy-2-[2-methyl-5-(3-propylpyrazol-1-yl)phenoxy]prop-2-enoate+TX, methyl (Z)-3-methoxy-2-[2-methyl-5-(4-propyltriazol-2-yl)phenoxy]prop-2-enoate+TX, methyl (Z)-3-methoxy-2-[2-methyl-5-[3-(trifluoromethyl)pyrazol-1-yl]phenoxy]prop-2-enoate (these compounds may be prepared from the methods described in WO2020/079111)+TX, methyl (Z)-3-methoxy-2-[2-methyl-5-[4-(trifluoromethyl)triazol-2-yl]phenoxy]prop-2-enoate+TX, methyl apholate+TX, methyl bromide+TX, methyl eugenol+TX, methyl isothiocyanate+TX, methyl N-[[4-[1-(2,6-difluoro-4-isopropyl-phenyl)pyrazol-4-yl]-2-methyl-phenyl]methyl]carbamate (may be prepared from the methods described in WO 2020/097012)+TX, methyl N-[[4-[1-(4-cyclopropyl-2,6-difluoro-phenyl)pyrazol-4-yl]-2-methyl-phenyl]methyl]carbamate (may be prepared from the methods described in WO 2020/097012)+TX, methyl N-[[5-[4-(2,4-dimethylphenyl)triazol-2-yl]-2-methyl-phenyl]methyl]carbamate+TX, methylchloroform+TX, methylene chloride+TX, methylneodecanamide+TX, metiram+TX, metolcarb+TX, metominostrobin+TX, metoxadiazone+TX, metrafenone+TX, metyltetraprole+TX, MGK 264+TX, milbemycin oxime+TX, mipafox+TX, mirex+TX, monocrotophos+TX, morphothion+TX, morzid+TX, moxidectin+TX, muscalure+TX, myclobutanil+TX, myclozoline+TX, Myrothecium verrucaria composition+TX, N-((1R)-1-benzyl-3-chloro-1-methyl-but-3-enyl)-8-fluoro-quinoline-3-carboxamide (these compounds may be prepared from the methods described in WO2017/153380)+TX, N-((1S)-1-benzyl-3-chloro-1-methyl-but-3-enyl)-8-fluoro-quinoline-3-carboxamide (these compounds may be prepared from the methods described in WO2017/153380)+TX, N′-(2,5-dimethyl-4-phenoxy-phenyl)-N-ethyl-N-methyl-formamidine+TX, N′-(2-chloro-5-methyl-4-phenoxy-phenyl)-N-ethyl-N-methyl-formamidine+TX, N,2-dimethoxy-N-[[4-[5-(trifluoromethyl)-1,2,4-oxadiazol-3-yl]phenyl]methyl]propanamide+TX, N,N-dimethyl-1-[[4-[5-(trifluoromethyl)-1,2,4-oxadiazol-3-yl]phenyl]methyl]-1,2,4-triazol-3-amine (THESE COMPOUNDS may be prepared from the methods described in WO 2017/055473, WO 2017/055469, WO 2017/093348 and WO 2017/118689)+TX, N-[(1R)-1-benzyl-1,3-dimethyl-butyl]-7,8-difluoro-quinoline-3-carboxamide+TX, N-[(1R)-1-benzyl-1,3-dimethyl-butyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-1,3-dimethyl-butyl]-7,8-difluoro-quinoline-3-carboxamide+TX, N-[(1S)-1-benzyl-1,3-dimethyl-butyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(E)-methoxyiminomethyl]-4-[5-(trifluoromethyl)-1,2,4-oxadiazol-3-yl]benzamide+TX, N-[(Z)-methoxyiminomethyl]-4-[5-(trifluoromethyl)-1,2,4-oxadiazol-3-yl]benzamide+TX, N-[[4-[5-(trifluoromethyl)-1,2,4-oxadiazol-3-yl]phenyl]methyl]propanamide+TX, N-[2-[2,4-dichloro-phenoxy]phenyl]-3-(difluoromethyl)-1-methyl-pyrazole-4-carboxamide+TX, N-[2-[2-chloro-4-(trifluoromethyl)phenoxy]phenyl]-3-(difluoromethyl)-1-methyl-pyrazole-4-carboxamide+TX, N′-[2-chloro-4-(2-fluorophenoxy)-5-methyl-phenyl]-N-ethyl-N-methyl-formamidine (this compound may be prepared from the methods described in WO 2016/202742)+TX, N′-[4-(4,5-dichlorothiazol-2-yl)oxy-2,5-dimethyl-phenyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine (these compounds may be prepared from the methods described in WO2015/155075)+TX, N′-[5-bromo-2-methyl-6-(2-propoxypropoxy)-3-pyridyl]-N-ethyl-N-methyl-formamidine (this compound may be prepared from the methods described in IPCOM000249876D)+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-[(1S)-1-methyl-2-propoxy-ethoxy]-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N-[N-methoxy-C-methyl-carbonimidoyl]-4-[5-(trifluoromethyl)-1,2,4-oxadiazol-3-yl]benzamide (these compounds may be prepared from the methods described in WO 2018/202428)+TX, N′-[4-(1-cyclopropyl-2,2,2-trifluoro-1-hydroxy-ethyl)-5-methoxy-2-methyl-phenyl]-N-isopropyl-N-methyl-formamidine (these compounds may be prepared from the methods described in WO2018/228896)+TX, nabam+TX, naftalofos+TX, naled+TX, naphthalene+TX, NC-170+TX, Neodiprion sertifer NPV and N. lecontei NPV+TX, nerolidol+TX, N-ethyl-2-methyl-N-[[4-[5-(trifluoromethyl)-1,2,4-oxadiazol-3-yl]phenyl]methyl]propanamide+TX, N-ethyl-N′-[5-methoxy-2-methyl-4-[(2-trifluoromethyl)oxetan-2-yl]phenyl]-N-methyl-formamidine+TX, nickel bis(dimethyldithiocarbamate)+TX, niclosamide-olamine+TX, nicotine+TX, nicotine sulfate+TX, nifluridide+TX, nikkomycins+TX, N-isopropyl-N′-[5-methoxy-2-methyl-4-(2,2,2-trifluoro-1-hydroxy-1-phenyl-ethyl)phenyl]-N-methyl-formamidine+TX, nithiazine+TX, nitrapyrin+TX, nitrilacarb+TX, nitrilacarb 1:1 zinc chloride complex+TX, nitrothal-isopropyl+TX, N-methoxy-N-[[4-[5-(trifluoromethyl)-1,2,4-oxadiazol-3-yl]phenyl]methyl]cyclopropanecarboxamide+TX, N-methyl-4-[5-(trifluoromethyl)-1,2,4-oxadiazol-3-yl]benzamide+TX, N-methyl-4-[5-(trifluoromethyl)-1,2,4-oxadiazol-3-yl]benzenecarbothioamide+TX, norbormide+TX, nuarimol+TX, O,O,O′,O′-tetrapropyl dithiopyrophosphate+TX, octadeca-2,13-dien-1-yl acetate+TX, octadeca-3,13-dien-1-yl acetate+TX, octhilinone+TX, ofurace+TX, oleic acid+TX, omethoate+TX, orfralure+TX, Orius spp.+TX, oryctalure+TX, orysastrobin+TX, ostramone+TX, oxadixyl+TX, oxamate+TX, oxathiapiprolin+TX, oxine-copper+TX, oxolinic acid+TX, oxycarboxin+TX, oxydeprofos+TX, oxydisulfoton+TX, oxytetracycline+TX, paclobutrazole+TX, Paecilomyces fumosoroseus+TX, para-dichlorobenzene+TX, parathion+TX, parathion-methyl+TX, pefurazoate+TX, penconazole+TX, pencycuron+TX, penflufen+TX, penfluron+TX, pentachlorophenol+TX, pentachlorophenyl laurate+TX, penthiopyrad+TX, permethrin+TX, PH 60-38+TX, phenamacril+TX, phenkapton+TX, phosacetim+TX, phosalone+TX, phosdiphen+TX, phosfolan+TX, phosglycin+TX, phosnichlor+TX, phosphamidon+TX, phosphine+TX, phosphorus+TX, phoxim-methyl+TX, phthalide+TX, Phytoseiulus persimilis+TX, picarbutrazox+TX, picaridin+TX, picoxystrobin+TX, pindone+TX, piperazine+TX, piperonyl butoxide+TX, piprotal+TX, pirimetaphos+TX, polychlorodicyclopentadiene isomers+TX, polychloroterpenes+TX, polynactins+TX, polyoxins+TX, potassium arsenite+TX, potassium ethylxanthate+TX, potassium hydroxyquinoline sulfate+TX, potassium thiocyanate+TX, pp′-DDT+TX, precocene I+TX, precocene II+TX, precocene Ill+TX, primidophos+TX, probenazole+TX, prochloraz+TX, proclonol+TX, procymidone+TX, profluthrin+TX, promacyl+TX, promecarb+TX, propamocarb+TX, propiconazole+TX, propineb+TX, propoxur+TX, propyl isomer+TX, proquinazid+TX, prothidathion+TX, prothioconazole+TX, prothiofos+TX, prothoate+TX, pydiflumetofen+TX, pyraclostrobin+TX, pyrametostrobin+TX, pyraoxystrobin+TX, pyrapropoyne+TX, pyraziflumid+TX, pyrazophos+TX, pyresmethrin+TX, pyrethrin I+TX, pyrethrin II+TX, pyrethrins+TX, pyribencarb+TX, pyridachlometyl+TX, pyridaphenthion+TX, pyridin-4-amine+TX, pyrifenox+TX, pyrimethanil+TX, pyrimitate+TX, pyrimorph+TX, pyrinuron+TX, pyriofenone+TX, pyrisoxazole+TX, pyroquilon+TX, Quassia+TX, quinalphos+TX, quinalphos-methyl+TX, quinoclamine+TX, quinofumelin+TX, quinonamid+TX, quinothion+TX, quinoxyfen+TX, quintiofos+TX, quintozene+TX, R-1492+TX, rafoxanide+TX, resmethrin+TX, Reynoutria sachalinensis extract+TX, ribavirin+TX, Rmetalaxyl+TX, rotenone+TX, ryania+TX, ryanodine+TX, S421+TX, sabadilla+TX, schradan+TX, scilliroside+TX, seboctylamine+TX, sebufos+TX, sedaxane+TX, selamectin+TX, sesamex+TX, sesasmolin+TX, SI-0009+TX, siglure+TX, simazine+TX, simeconazole+TX, sodium arsenite+TX, sodium cyanide+TX, sodium fluoride+TX, sodium fluoroacetate+TX, sodium hexafluorosilicate+TX, sodium pentachlorophenoxide+TX, sodium selenate+TX, sodium tetrathiocarbonate+TX, sodium thiocyanate+TX, sophamide+TX, sordidin+TX, spiroxamine+TX, SSI-121+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, streptomycin+TX, streptomycin sesquisulfate+TX, strychnine+TX, sulcatol+TX, sulcofuron+TX, sulcofuron-sodium+TX, sulfiram+TX, sulfluramid+TX, sulfotep+TX, sulfoxide+TX, sulfur+TX, sulfuryl fluoride+TX, sulprofos+TX, tar oils+TX, tau-fluvalinate+TX, tazimcarb+TX, TDE+TX, tebuconazole+TX, tebufloquin+TX, tebupirimfos+TX, tecloftalam+TX, temephos+TX, tepa+TX, TEPP+TX, terallethrin+TX, terbam+TX, tert-butyl N-[6-[[[(1-methyltetrazol-5-yl)-phenyl-methylene]amino]oxymethyl]-2-pyridyl]carbamate+TX, tetrachloroethane+TX, tetrachlorothiophene+TX, tetraconazole+TX, tetradec-11-en-1-yl acetate+TX, tetradifon+TX, tetramethylfluthrin+TX, tetrasul+TX, thallium sulfate+TX, thiabendazole+TX, thiafenox+TX, thiapronil+TX, thicrofos+TX, thifluzamide+TX, thiocarboxime+TX, thiocyclam+TX, thiocyclam hydrogen oxalate+TX, thiodiazole copper+TX, thiofanox+TX, thiohempa+TX, thiomersal+TX, thiometon+TX, thionazin+TX, thiophanate+TX, thiophanate-methyl+TX, thioquinox+TX, thiosultap+TX, thiosultap-sodium+TX, thiotepa+TX, thiram+TX, thuringiensin+TX, tiadinil+TX, tolclofos-methyl+TX, tolprocarb+TX, tolylfluanid+TX, tralomethrin+TX, transpermethrin+TX, tretamine+TX, triadimefon+TX, triadimenol+TX, triamiphos+TX, triarathene+TX, triazamate+TX, triazophos+TX, triazoxide+TX, triazuron+TX, tributyltin oxide+TX, trichlormetaphos-3+TX, trichloronat+TX, Trichogramma spp.+TX, triclopyricarb+TX, tricyclazole+TX, tridemorph+TX, trifenmorph+TX, trifenofos+TX, trifloxystrobin+TX, triflumizole+TX, triforine+TX, trimedlure+TX, trimedlure A+TX, trimedlure B1+TX, trimedlure B2+TX, trimedlure C+TX, trimethacarb+TX, trinactin+TX, trinexapac+TX, triphenyltin acetate+TX, triphenyltin hydroxide+TX, triprene+TX, triticonazole+TX, trunc-call+TX, Typhlodromus occidentalis+TX, uredepa+TX, validamycin+TX, valifenalate+TX, vamidothion+TX, vaniliprole+TX, veratridine+TX, veratrine+TX, verbutin+TX, Verticillium lecanii+TX, vinclozoline+TX, warfarin+TX, XMC+TX, xylenols+TX, zeatin+TX, zetamethrin+TX, zhongshengmycin+TX, zinc naphthenate+TX, zinc phosphide+TX, zinc thiazole+TX, zineb+TX, ziram+TX, zolaprofos+TX;
Acinetobacter lwoffii+TX, Acremonium alternatum+TX, Acremonium cephalosporium+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum (MicroAZ®, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain AQ746+TX, Bacillus chitinosporus strain CM-1+TX, Bacillus circulans+TX, Bacillus firmus (BioSafe®, BioNem-WP®) in particular strain CNMC 1-1582 (e.g. VOTIVO® from BASF SE)+TX, Bacillus licheniformis strain 3086 (EcoGuard®, Green Releaf®)+TX, Bacillus licheniformis strain HB-2 (Biostart™ formerly Rhizoboost®)+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain AQ717+TX, Bacillus pumilus strain GB34 (Yield Shield®)+TX, Bacillus pumilus strain QST 2808 (Sonata®, Ballad Plus®)+TX, Bacillus sphaericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain AQ153+TX, Bacillus subtilis strain AQ743+TX, Bacillus subtilis strain QST 713 (CEASE®, Serenade®, Rhapsody®)+TX, Bacillus subtilis strain QST 714 (JAZZ®)+TX, Bacillus subtilis strain QST3002+TX, Bacillus subtilis strain QST3004+TX, Bacillus subtilis var. amyloliquefaciens strain FZB24 (Taegro®, Rhizopro®)+TX, Bacillus thuringiensis aizawai GC 91 (Agree®)+TX, Bacillus thuringiensis Cry 2Ae+TX, Bacillus thuringiensis Cry1Ab+TX, Bacillus thuringiensis israelensis (BMP123®, Aquabac®, VectoBac®)+TX, Bacillus thuringiensis kurstaki (Javelin®, Deliver®, CryMax®, Bonide®, Scutella WP®, Turilav WP®, Astuto®, Dipel WP®, Biobit®, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain AQ52+TX, Bacillus thuringiensis strain BD #32+TX, Bacillus thuringiensis tenebrionis (Novodor®, BtBooster)+TX, Bacillus thuringiensis var. aizawai (XenTari®, DiPel®)+TX, bacteria spp. (GROWMEND®, GROWSWEET®, Shootup®)+TX, bacteriophage of Clavipacter michiganensis (AgriPhage®, Bakflor®)+TX, Beauveria bassiana (Beaugenic®, Brocaril WP®)+TX, Beauveria bassiana GHA (Mycotrol ES®, Mycotrol O®, BotaniGuard®)+TX, Beauveria brongniartii (Engerlingspilz®, Schweizer Beauveria®, Melocont®)+TX, Beauveria spp.+TX, Botrytis cineria+TX, Bradyrhizobium japonicum (TerraMax®)+TX, Brevibacillus brevis+TX, Burkholderia cepacia (Deny®, Intercept®, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®, Biocure®)+TX, Candida sake+TX, Candida spp.+TX, Candida tenius+TX, Cedecea davisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium chlorocephalum+TX, Cladosporium cladosporioides+TX, Cladosporium oxysporum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmo-miniatus+TX, Cryptococcus laurentii+TX, Cryptophlebia leucotreta granulovirus (Cryptex®)+TX, Cupriavidus campinensis+TX, Cydia pomonella granulovirus (CYD-X®, Madex®, Madex® Plus, Madex Max, Carpovirusine®+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®, Biofox C®)+TX, Fusarium proliferatum+TX, Fusarium spp.+TX, Galactomyces geotrichum+TX, Gliocladium catenulatum (Primastop®, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isaria fumosorosea (previously known as Paecilomyces fumosoroseus strain, PFR-97®, PreFeRal®)+TX, Isoflavone formononetin (Myconate®)+TX, Kloeckera apiculata+TX, Kloeckera spp.+TX, Lagenidium giganteum (Laginex®)+TX, Lecanicillium lecanii (formerly known as Verticillium lecanii (Mycotal®) conidia of strain KV01 (e.g. Vertalec® by Koppert/Arysta)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Destruxin WP®)+TX, Metarhizium anisopliae (Met52®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus in particular strain A3-5 (Accession No. NRRL 30548)+TX, Mycorrhizae spp. (AMykor®, Root Maximizer®)+TX, Myrothecium verrucaria strain AARC-0255 (DiTera®, BROS PLUS®)+TX, Ophiostoma piliferum strain D97 (Sylvanex®)+TX, Paecilomyces farinosus+TX, Paecilomyces lilacinus strain 251 (MeloCon WG®)+TX, Paecilomyces linacinus (Biostat WP®)+TX, Paenibacillus polymyxa+TX, Pantoea agglomerans (BlightBan C9-1®)+TX, Pantoea spp.+TX, Pasteuria nishizawae in particular strain Pn1 (CLARIVA from Syngenta/ChemChina); +TX, Pasteuria spp. (Econem®)+TX, Penicillium aurantiogriseum+TX, Penicillium billai (Jumpstart®, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilliermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens (Zequanox®)+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®, Polyversum®)+TX, Pythium periplocum+TX, Rhanella aquatilis+TX, Rhanella spp.+TX, Rhizobia (Dormal®, Vault®)+TX, Rhizoctonia+TX, Rhodococcus globerulus strain AQ719+TX, Rhodosporidium diobovatum+TX, Rhodosporidium toruloides+TX, Rhodotorula glutinis+TX, Rhodotorula graminis+TX, Rhodotorula mucilagnosa+TX, Rhodotorula rubra+TX, Rhodotorula spp.+TX, Saccharomyces cerevisiae+TX, Salinococcus roseus+TX, Sclerotinia minor(SARRITOR®)+TX, Sclerotinia minor+TX, Scytalidium spp.+TX, Scytalidium uredinicola+TX, Serratia marcescens+TX, Serratia plymuthica+TX, Serratia spp.+TX, Sordaria fimicola+TX, Spodoptera exigua nuclear polyhedrosis virus (Spod-X®, Spexit®)+TX, Spodoptera littoralis nucleopolyhedrovirus (Littovir®)+TX, Sporobolomyces roseus+TX, Stenotrophomonas maltophilia+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces hygroscopicus+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma atroviride (Plantmate®)+TX, Trichoderma gamsii (Tenet®)+TX, Trichoderma hamatum TH 382+TX, Trichoderma harzianum rifai (Mycostar®)+TX, Trichoderma harzianum T-22 (Trianum-P®, PlantShield HCO, RootShield®, Trianum-G®+TX, Trichoderma harzianum T-39 (Trichodex®)+TX, Trichoderma inhamatum+TX, Trichoderma koningii+TX, Trichoderma lignorum+TX, Trichoderma longibrachiatum+TX, Trichoderma polysporum (Binab T®)+TX, Trichoderma spp. LC 52 (Sentinel®)+TX, Trichoderma taxi+TX, Trichoderma virens (formerly Gliocladium virens GL-21) (SoilGuard®)+TX, Trichoderma virens+TX, Trichoderma viride+TX, Trichoderma viride strain ICC 080 (Remedier®)+TX, Trichosporon pullulans+TX, Trichosporon spp. +TX, Trichothecium roseum+TX, Trichothecium spp.+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Vip3Aa20 (VlPtera®)+TX, Virgibaclillus marismortui+TX, Xanthomonas campestris pv. Poae (Camperico®)+TX, Xenorhabdus bovienii+TX, Xenorhabdus nematophilus+TX;

AGNIQUE® MMF+TX, azadirachtin (Plasma Neem Oil®, AzaGuard®, MeemAzal®, Molt-X® e.g. AZATIN XL from Certis, US)+TX, Botanical IGR (Neemazad®, Neemix®)+TX, BugOil®+TX, canola oil (Lilly Miller Vegol®)+TX, Chenopodium ambrosioides near ambrosioides (Requiem®)+TX, Chrysanthemum extract (Crisant®)+TX, essentials oils of Labiatae (Botania®)+TX, extract of neem oil (Trilogy®)+TX, extracts of clove rosemary peppermint and thyme oil (Garden insect killer®)+TX, garlic+TX, Glycinebetaine (Greenstim®)+TX, kaolin (Screen®)+TX, lemongrass oil (GreenMatch®)+TX, Melaleuca alternifolia extract (also called tea tree oil) (Timorex Gold®)+TX, mixture of clove peppermint garlic oil and mint (Soil Shot®)+TX, mixture of clove rosemary and peppermint extract (EF 400®)+TX, mixture of rosemary sesame peppermint thyme and cinnamon extracts (EF 300®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pine oil (Retenol®)+TX, pyrethrum+TX, Quillaja saponaria (NemaQ®)+TX, Reynoutria sachalinensis (Regalia®, Sakalia®)+TX, rotenone (Eco Roten®)+TX, Rutaceae plant extract (Soleo®)+TX, soybean oil (Ortho ecosense®)+TX, storage glucam of brown algae (Laminarin®)+TX, thyme oil+TX; (E,Z)-7,9-Dodecadien-1-yl acetate+TX, (E,Z,Z)-3,8,11 Tetradecatrienyl acetate+TX, (Z,Z,E)-7,11,13-Hexadecatrienal+TX, 2-Methyl-1-butanol+TX, Biolure®+TX, blackheaded fireworm pheromone (3M Sprayable Blackheaded Fireworm Pheromone®)+TX, Calcium acetate+TX, Check-Mate®+TX, Codling Moth Pheromone (Paramount dispenser-(CM)/Isomate C-Plus®)+TX, Entostat powder (extract from palm tree) (Exosex CM®)+TX, Grape Berry Moth Pheromone (3M MEC-GBM Sprayable Pheromone®)+TX, Lavandulyl senecioate+TX, Leafroller pheromone (3M MEC-LR Sprayable Pheromone®)+TX, Muscamone (Snip7 Fly Bait®+TX, Oriental Fruit Moth Pheromone (3M oriental fruit moth sprayable pheromone®)+TX, Peachtree Borer Pheromone (Isomate-P®)+TX, Scenturion®+TX, Starbar Premium Fly Bait®)+TX, Tomato Pinworm Pheromone (3M Sprayable pheromone®)+TX; Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®, Andersoni-System®)+TX, Amblyseius californicus (Amblyline®, Spical®)+TX, Amblyseius cucumeris (Thripex®, Bugline cucumeris®)+TX, Amblyseius fallacis (Fallacis®)+TX, Amblyseius swirskii (Bugline swirskii®, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®, Aphiline®), +TX, Aphelinus asychis+TX, Aphidius colemani (Aphipar®)+TX, Aphidius ervi (Aphelinus-System®)+TX, Aphidius ervi (Ervipar®)+TX, Aphidius gifuensis+TX, Aphidius matricariae (Aphipar-M®)+TX, Aphidoletes aphidimyza (Aphidend®, Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Beeline®, Tripol®)+TX, Bombus terrestris (Natupol Beehive®)+TX, Cephalonomia stephanoderis+TX, Chilocorus nigritus+TX, Chrysoperla carnea (Chrysoline®, Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®, Cryptoline®)+TX, Cybocephalus nipponicus+TX, Dacnusa sibirica (Minusa®, DacDigline®, Minex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea (Diminex®, Miglyphus®, Digline®)+TX, Diversinervus spp.+TX, Encarsia citrina+TX, Encarsia formosa (Encarsia max®, Encarline®, En-Strip®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Enermix®, Ercal®, Eretline e®, Bemimix®)+TX, Eretmocerus hayati+TX, Eretmocerus mundus (Bemipar®, Eretline m®)+TX, Eretmocerus siphonini+TX, Exochomus quadripustulatus+TX, Feltiella acarisuga (Feltiline®)+TX, Feltiella acarisuga (Spidend®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis bacteriophora (NemaShield HB®, Nemaseek®, Terranem-Nam®, Terranem®, Larvanem®, B-Green®, NemAttack®, Nematop®)+TX, Heterorhabditis megidis (Nemasys H®, BioNem H®, Exhibitline hm®, Larvanem-M®)+TX, Heterorhabditis spp. (Lawn Patrol®)+TX, Hippodamia convergens+TX, Hypoaspis aculeifer (Aculeifer-System®, Entomite-A®)+TX, Hypoaspis miles (Hypoline m®, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®, Macroline c®, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®, Nesibug®)+TX, Ophyra aenescens (Biofly®)+TX, Orius insidiosus (Thripor-I®, Oriline i®)+TX, Orius laevigatus (Thripor-L®, Oriline I®)+TX, Orius majusculus (Oriline m®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®, Phytoline p®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®, Millenium®, BioNem C®, NemAttack®, Nemastar®, Capsanem®)+TX, Steinernema feltiae (NemaShield®, Nemasys F®, BioNem F®, Steinernema-System®, NemAttack®, Nemaplus®, Exhibitline sf®, Scia-rid®, Entonem®)+TX, Steinernema kraussei (Nemasys L®, BioNem L®, Exhibitline srb®)+TX, Steinernema riobrave (BioVector®, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer4+TX, Thripobius semiluteus+TX, Torymus sinensis+TX, Trichogramma brassicae (Tricholine b®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator+TX;
abscisic acid+TX, Aminomite®+TX, BioGain®+TX, bioSea®+TX, CAS Number: 2643947-26-4+TX, Chondrostereum purpureum (Chontrol Paste®)+TX, Colletotrichum gloeosporioides (Collego®)+TX, Copper Octanoate (Cueva®)+TX, Delta traps (Trapline d®)+TX, Erwinia amylovora (Harpin) (ProAct®, Ni-HIBIT Gold CST®)+TX, fatty acids derived from a natural by-product of extra virgin olive oil (FLIPPER®)+TX, Ferri-phosphate (Ferramol®)+TX, Funnel traps (Trapline y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline f®)+TX, Microctonus hyperodae+TX, Mycoleptodiscus terrestris (Des-X®)+TX, Nosema locustae (Semaspore Organic Grasshopper Control®)+TX, Pheromone trap (Thripline ams®)+TX, potassium bicarbonate (MilStop®)+TX, potassium iodide+potassiumthiocyanate (Enzicur®)+TX, potassium salts of fatty acids (Sanova®)+TX, potassium silicate solution (Sil-Matrix®)+TX, Spider venom+TX, Sticky traps (Trapline YF®, Rebell Amarillo®)+TX, SuffOil-X®+TX, Traps (Takitrapline y+b®)+TX; Bacillus mojavensis strain R3B (Accession No. NCAIM (P) B001389) (WO 2013/034938) from Certis USA LLC+TX, Bacillus pumilus, in particular strain BU F-33, having NRRL Accession No. 50185 (CARTISSA® from BASF, EPA Reg. No. 71840-19)+TX, Bacillus subtilis CX-9060 from Certis USA LLC, Bacillus sp., in particular strain D747 (available as DOUBLE NICKEL® from Kumiai Chemical Industry Co., Ltd.), having Accession No. FERM BP-8234, U.S. Pat. No. 7,094,592+TX, Bacillus subtilis strain BU1814, (VELONDIS® PLUS, VELONDIS® FLEX and VELONDIS® EXTRA from BASF SE)+TX, Bacillus subtilis var. amyloliquefaciens strain FZB24 having Accession No. DSM 10271 (available from Novozymes as TAEGRO® or TAEGRO® ECO (EPA Registration No. 70127-5))+TX, Bacillus subtilis, in particular strain QST713/AQ713 (having NRRL Accession No. B-21661 and described in U.S. Pat. No. 6,060,051, available as SERENADE® OPTI or SERENADE® ASO from Bayer CropScience LP, US)+TX, Paenibacillus polymyxa, in particular strain AC-1 (e.g. TOPSEED® from Green Biotech Company Ltd.)+TX, Paenibacillus sp. strain having Accession No. NRRL B-50972 or Accession No. NRRL B-67129, WO 2016/154297+TX, Pantoea agglomerans, in particular strain E325 (Accession No. NRRL B-21856) (available as BLOOMTIME BIOLOGICAL™ FD BIOPESTICIDE from Northwest Agri Products)+TX, Pseudomonas proradix (e.g. PRORADIX® from Sourcon Padena)+TX;
Aureobasidium pullulans, in particular blastospores of strain DSM14940, blastospores of strain DSM 14941 or mixtures of blastospores of strains DSM14940 and DSM14941 (e.g., BOTECTOR® and BLOSSOM PROTECT® from bio-ferm, CH)+TX, Pseudozyma aphidis (as disclosed in WO2011/151819 by Yissum Research Development Company of the Hebrew University of Jerusalem)+TX, Saccharomyces cerevisiae, in particular strains CNCM No. 1-3936, CNCM No. 1-3937, CNCM No. 1-3938 or CNCM No. 1-3939 (WO 2010/086790) from Lesaffre et Compagnie, FR+TX; Agrobacterium radiobacter strain K84 (e.g. GALLTROL-A® from AgBioChem, CA)+TX, Bacillus amyloliquefaciens isolate B246 (e.g. AVOGREEN™ from University of Pretoria)+TX, Bacillus amyloliquefaciens strain F727 (also known as strain MB1110) (NRRL Accession No. B-50768, WO 2014/028521) (STARGUS® from Marrone Bio Innovations)+TX, Bacillus amyloliquefaciens strain FZB42, Accession No. DSM 23117 (available as RHIZOVITAL® from ABiTEP, DE)+TX, Bacillus amyloliquefaciens, in particular strain D747 (available as Double Nickel™ from Kumiai Chemical Industry Co., Ltd., having accession number FERM BP-8234, U.S. Pat. No. 7,094,592)+TX, Bacillus licheniformis FMCH001 and Bacillus subtilis FMCH002 (QUARTZO® (WG) and PRESENCE® (WP) from FMC Corporation)+TX, Bacillus licheniformis, in particular strain SB3086, having Accession No. ATCC 55406, WO 2003/000051 (available as ECOGUARD® Biofungicide and GREEN RELEAF™ from Novozymes)+TX, Bacillus methylotrophicus strain BAC-9912 (from Chinese Academy of Sciences' Institute of Applied Ecology)+TX, Bacillus mycoides, isolate, having Accession No. B-30890 (available as BMJ TGAI® or WG and LifeGard™ from Certis USA LLC)+TX, Bacillus pumilus, in particular strain GB34 (available as Yield Shield® from Bayer AG, DE)+TX, Bacillus pumilus, in particular strain QST2808 (available as SONATA® from Bayer CropScience LP, US, having Accession No. NRRL B-30087 and described in U.S. Pat. No. 6,245,551)+TX, Bacillus subtilis CX-9060 from Certis USA LLC+TX, Bacillus subtilis IAB/BS03 (AVIV™ from STK Bio-Ag Technologies, PORTENTO® from Idai Nature)+TX, Bacillus subtilis KTSB strain (FOLIACTIVE® from Donaghys)+TX, Bacillus subtilis strain BU1814, (available as VELONDIS® PLUS, VELONDIS® FLEX and VELONDIS® EXTRA from BASF SE)+TX, Bacillus subtilis strain GB03 (available as Kodiak® from Bayer AG, DE)+TX, Bacillus subtilis strain MBI 600 (available as SUBTILEX from BASF SE), having Accession Number NRRL B-50595, U.S. Pat. No. 5,061,495+TX, Bacillus subtilis strain Y1336 (available as BIOBAC® WP from Bion-Tech, Taiwan, registered as a biological fungicide in Taiwan under Registration Nos. 4764, 5454, 5096 and 5277)+TX, Bacillus subtilis var. amyloliquefaciens strain FZB24 having Accession No. DSM 10271 (available from Novozymes as TAEGRO® or TAEGRO® ECO (EPA Registration No. 70127-5))+TX, Bacillus subtilis Y1336 (available as BIOBAC® WP from Bion-Tech, Taiwan, registered as a biological fungicide in Taiwan under Registration Nos. 4764, 5454, 5096 and 5277)+TX, Paenibacillus epiphyticus (WO 2016/020371) from BASF SE+TX, Paenibacillus polymyxa ssp. plantarum (WO 2016/020371) from BASF SE+TX, Paenibacillus sp. strain having Accession No. NRRL B-50972 or Accession No. NRRL B-67129, WO 2016/154297+TX, Pseudomonas chlororaphis strain AFS009, having Accession No. NRRL B-50897, WO 2017/019448 (e.g., HOWLER™ and ZIO® from AgBiome Innovations, US)+TX, Pseudomonas chlororaphis, in particular strain MA342 (e.g. CEDOMON®, CERALL®, and CEDRESS® by Bioagri and Koppert)+TX, Pseudomonas fluorescens strain A506 (e.g. BLIGHTBAN® A506 by NuFarm)+TX, Pseudomonas proradix (e.g. PRORADIX® from Sourcon Padena)+TX, Streptomyces griseoviridis strain K61 (also known as Streptomyces galbus strain K61) (Accession No. DSM 7206) (MYCOSTOP® from Verdera, PREFENCE® from BioWorks, cf. Crop Protection 2006, 25, 468-475)+TX, Streptomyces lydicus strain WYEC108 (also known as Streptomyces lydicus strain WYCD108US) (ACTINO-IRON® and ACTINOVATE® from Novozymes)+TX; Trichoderma atroviride strain T11 (IM1352941/CECT20498)+TX, Ampelomyces quisqualis strain AQ10, having Accession No. CNCM 1-807 (e.g., AQ 10® by IntrachemBio Italia)+TX, Ampelomyces quisqualis, in particular strain AQ 10 (e.g. AQ 10® by IntrachemBio Italia)+TX, Aspergillus flavus strain NRRL 21882 (products known as AFLA-GUARD® from Syngenta/ChemChina)+TX, Aureobasidium pullulans, in particular blastospores of strain DSM 14941+TX, Aureobasidium pullulans, in particular blastospores of strain DSM14940+TX, Aureobasidium pullulans, in particular mixtures of blastospores of strains DSM14940 and DSM 14941 (e.g. Botector® by bio-ferm, CH)+TX, Chaetomium cupreum (Accession No. CABI 353812) (e.g. BIOKUPRUM™ by AgriLife)+TX, Chaetomium globosum (available as RIVADIOM® by Rivale)+TX, Cladosporium cladosporioides, strain H39, having Accession No. CBS122244, US 2010/0291039 (by Stichting Dienst Landbouwkundig Onderzoek)+TX, Coniothyrium minitans, in particular strain CON/M/91-8 (Accession No. DSM9660, e.g. Contans® from Bayer CropScience Biologics GmbH)+TX, Cryptococcus flavescens, strain 3C (NRRL Y-50378), +TX, Dactylaria candida, Dilophosphora alopecuri (available as TWIST FUNGUS®), Fusarium oxysporum, strain Fo47 (available as FUSACLEAN® by Natural Plant Protection)+TX, Gliocladium catenulatum (Synonym: Clonostachys rosea f. catenulate) strain J1446 (e.g. Prestop® by Lallemand)+TX, Gliocladium roseum (also known as Clonostachys rosea f rosea) strain IK726 (Jensen D F, et al. Development of a biocontrol agent for plant disease control with special emphasis on the near commercial fungal antagonist Clonostachys rosea strain ‘IK726’, Australasian Plant Pathol. 2007,36(2):95-101)+TX, Gliocladium roseum (also known as Clonostachys rosea f rosea), in particular strain 321U from Adjuvants Plus, strain ACM941 as disclosed in Xue A. G. (Efficacy of Clonostachys rosea strain ACM941 and fungicide seed treatments for controlling the root tot complex of field pea, Can Jour Plant Sci 2003, 83(3): 519-524)+TX, Metschnikowia fructicola, in particular strain NRRL Y-30752+TX, Microsphaeropsis ochracea, Penicillium steckii (DSM 27859, WO 2015/067800) from BASF SE+TX, mixtures of Trichoderma asperellum strain ICC 012 (also known as Trichoderma harzianum ICC012), having Accession No. CABI CC IMI 392716 and Trichoderma gamsii (formerly T. viride) strain ICC 080, having Accession No. IMI 392151 (e.g., BIO-TAM™ from Isagro USA, Inc. or BIODERMA® by Agrobiosol de Mexico, S.A. de C.V.)+TX, Penicillium vermiculatum+TX, Phlebiopsis gigantea strain VRA 1992 (ROTSTOP® C from Danstar Ferment)+TX, Pseudozyma flocculosa, strain PF-A22 UL (available as SPORODEX® L by Plant Products Co., CA)+TX, Saccharomyces cerevisiae strain LAS117 cell walls (CEREVISANE® from Lesaffre, ROMEO® from BASF SE)+TX, Saccharomyces cerevisiae strains CNCM No. 1-3936, CNCM No. 1-3937, CNCM No. 1-3938, CNCM No. 1-3939 (WO 2010/086790) from Lesaffre et Compagnie, FR+TX, Saccharomyces cerevisiae, in particular strain LASO2 (from Agro-Levures et Derives)+TX, Simplicillium lanosoniveum+TX, strain T34 (e.g. T34 Biocontrol by Biocontrol Technologies S.L., ES) or strain ICC 012 from Isagro+TX, strain WRL-076 (NRRL Y-30842), U.S. Pat. No. 7,579,183+TX, Talaromyces flavus, strain V117b+TX, Trichoderma asperelloides JM41R (Accession No. NRRL B-50759) (TRICHO PLUS® from BASF SE)+TX, Trichoderma asperellum, in particular strain SKT-1, having Accession No. FERM P-16510 (e.g. ECO-HOPE® from Kumiai Chemical Industry)+TX, Trichoderma asperellum, in particular, strain kd (e.g. T-Gro from Andermatt Biocontrol)+TX, Trichoderma atroviride strain 77B (T77 from Andermatt Biocontrol)+TX, Trichoderma atroviride strain ATCC 20476 (IMI 206040)+TX, Trichoderma atroviride strain LC52 (e.g. Tenet by Agrimm Technologies Limited)+TX, Trichoderma atroviride strain LU132 (e.g. Sentinel from Agrimm Technologies Limited)+TX, Trichoderma atroviride strain NMI no. V08/002388+TX, Trichoderma atroviride strain NMI no. V08/002389+TX, Trichoderma atroviride strain NMI no. V08/002390+TX, Trichoderma atroviride strain no. V08/002387+TX, Trichoderma atroviride strain SKT-1 (FERM P-16510), JP Patent Publication (Kokai) 11-253151 A+TX, Trichoderma atroviride strain SKT-2 (FERM P-16511), JP Patent Publication (Kokai) 11-253151 A+TX, Trichoderma atroviride strain SKT-3 (FERM P-17021), JP Patent Publication (Kokai) 11-253151 A+TX, Trichoderma atroviride, in particular strain SC1 (Accession No. CBS 122089, WO 2009/116106 and U.S. Pat. No. 8,431,120 (from Bi-PA))+TX, Trichoderma atroviride, strain CNCM 1-1237 (e.g. Esquive® WP from Agrauxine, FR)+TX, Trichoderma fertile (e.g. product TrichoPlus from BASF)+TX, Trichoderma gamsii (formerly T. viride)+TX, Trichoderma gamsii (formerly T. viride) strain ICC 080 (IMI CC 392151 CABI) (available as BIODERMA® by AGROBIOSOL DE MEXICO, S.A. DE C.V.), +TX, Trichoderma gamsii strain ICCO80 (IMI CC 392151 CABI, e.g. BioDerma by AGROBIOSOL DE MEXICO, S.A. DE C.V.), +TX, Trichoderma harmatum+TX, Trichoderma harmatum, having Accession No. ATCC 28012+TX, Trichoderma harzianum+TX, Trichoderma harzianum rifai T39 (e.g. Trichodex® from Makhteshim, US)+TX, Trichoderma harzianum strain Cepa SimbT5 (from Simbiose Agro), +TX, Trichoderma harzianum strain DB 103 (available as T-GRO® 7456 by Dagutat Biolab)+TX, Trichoderma harzianum strain ITEM 908 (e.g. Trianum-P from Koppert)+TX, Trichoderma harzianum strain T-22 (e.g. Trianum-P from Andermatt Biocontrol or Koppert)+TX, Trichoderma harzianum strain TH35 (e.g. Root-Pro by Mycontrol)+TX, Trichoderma polysporum strain IMI 206039 (e.g. Binab TF WP by BINAB Bio-Innovation AB, Sweden)+TX, Trichoderma stromaticum having Accession No. Ts3550 (e.g. Tricovab by CEPLAC, Brazil)+TX, Trichoderma virens (also known as Gliocladium virens) in particular strain GL-21 (e.g. SoilGard by Certis, US)+TX, Trichoderma virens strain G-41, formerly known as Gliocladium virens (Accession No. ATCC 20906) (e.g., ROOTSHIELD® PLUS WP and TURFSHIELD® PLUS WP from BioWorks, US)+TX, Trichoderma viride in particular strain B35 (Pietr et al., 1993, Zesz. Nauk. A R w Szczecinie 161: 125-137)+TX, Trichoderma viride strain TV1(e.g. Trianum-P by Koppert)+TX, Ulocladium oudemansii strain U3, having Accession No. NM 99/06216 (e.g., BOTRY-ZEN® by Botry-Zen Ltd, New Zealand and BOTRYSTOP® from BioWorks, Inc.)+TX, Verticillium albo-atrum (formerly V. dahliae) strain WCS850 having Accession No. WCS850, deposited at the Central Bureau for Fungi Cultures (e.g., DUTCH TRIG® by Tree Care Innovations)+TX, Verticillium chlamydosporium+TX;
a mixture of Azotobacter vinelandii and Clostridium pasteurianum (available as INVIGORATE® from Agrinos)+TX, a mixture of Bacillus licheniformis FMCH001 and Bacillus subtilis FMCH002 (available as QUARTZO® (WG), PRESENCE® (WP) from FMC Corporation)+TX, Azorhizobium caulinodans, in particular strain ZB-SK-5+TX, Azospirillum brasilense (e.g., VIGOR® from KALO, Inc.)+TX, Azospirillum lipoferum (e.g., VERTEX-IF™ from TerraMax, Inc.)+TX, Azotobacter chroococcum, in particular strain H23+TX, Azotobacter vinelandii, in particular strain ATCC 12837+TX, Bacillus amyloliquefaciens BS27 (Accession No. NRRL B-5015)+TX, Bacillus amyloliquefaciens in particular strain FZB42 (e.g. RHIZOVITAL® from ABiTEP, DE)+TX, Bacillus amyloliquefaciens in particular strain IN937a+TX, Bacillus amyloliquefaciens pm414 (LOLI-PEPTA® from Biofilm Crop Protection)+TX, Bacillus amyloliquefaciens SB3281 (ATCC #PTA-7542, WO 2017/205258)+TX, Bacillus amyloliquefaciens TJ1000 (available as QUIKROOTS® from Novozymes)+TX, Bacillus cereus family member EE128 (NRRL No. B-50917)+TX, Bacillus cereus family member EE349 (NRRL No. B-50928)+TX, Bacillus cereus in particular strain BP01 (ATCC 55675, e.g. MEPICHLOR® from Arysta Lifescience, US)+TX, Bacillus mycoides BT155 (NRRL No. B-50921)+TX, Bacillus mycoides BT46-3 (NRRL No. B-50922)+TX, Bacillus mycoides EE118 (NRRL No. B-50918)+TX, Bacillus mycoides EE141 (NRRL No. B-50916)+TX, Bacillus pumilus in particular strain GB34 (e.g. YIELD SHIELD® from Bayer Crop Science, DE), +TX, Bacillus pumilus in particular strain QST2808 (Accession No. NRRL No. B-30087)+TX, Bacillus siamensis in particular strain KCTC 13613T+TX, Bacillus subtilis in particular strain AQ30002 (Accession No. NRRL No. B-50421 and described in U.S. patent application Ser. No. 13/330,576)+TX, Bacillus subtilis in particular strain AQ30004 (NRRL No. B-50455 and described in U.S. patent application Ser. No. 13/330,576)+TX, Bacillus subtilis in particular strain MBI 600 (e.g. SUBTILEX® from BASF SE)+TX, Bacillus subtilis rm303 (RHIZOMAX® from Biofilm Crop Protection)+TX, Bacillus subtilis strain BU1814 (available as TEQUALIS® from BASF SE)+TX, Bacillus tequilensis in particular strain NII-0943+TX, Bacillus thuringiensis BT013A (NRRL No. B-50924) also known as Bacillus thuringiensis 4Q7+TX, Bradyrhizobium japonicum (e.g. OPTIMIZE® from Novozymes)+TX, Delftia acidovorans in particular strain RAY209 (e.g. BIOBOOST® from Brett Young Seeds)+TX, Lactobacillus sp. (e.g. LACTOPLANT® from LactoPAFI)+TX, Mesorhizobium cicer (e.g., NODULATOR from BASF SE)+TX, Paenibacillus polymyxa in particular strain AC-1 (e.g. TOPSEED® from Green Biotech Company Ltd.)+TX, Pseudomonas aeruginosa in particular strain PN1+TX, Pseudomonas proradix (e.g. PRORADIX® from Sourcon Padena)+TX, Rhizobium leguminosarium biovar viciae (e.g., NODULATOR from BASF SE)+TX, Rhizobium leguminosarum in particular bv. viceae strain Z25 (Accession No. CECT 4585)+TX, Serratia marcescens in particular strain SRM (Accession No. MTCC 8708), +TX, Sinorhizobium meliloti strain NRG-185-1 (NITRAGIN® GOLD from Bayer CropScience)+TX, Thiobacillus sp. (e.g. CROPAID® from Cropaid Ltd UK)+TX; Myrothecium verrucaria strain AARC-0255 (e.g. DiTera™ from Valent Biosciences)+TX, Penicillium bilaii strain ATCC 22348 (e.g. JumpStart® from Acceleron BioAg)+TX, Penicillium bilaii strain ATCC ATCC20851+TX, Purpureocillium lilacinum (previously known as Paecilomyces lilacinus) strain 251 (AGAL 89/030550, e.g. BioAct from Bayer CropScience Biologics GmbH)+TX, Pythium oligandrum strain DV74+TX, Pythium oligandrum strain M1 (ATCC 38472 e.g. Polyversum from Bioprepraty, CZ)+TX, Rhizopogon amylopogon (Myco-Sol from Agri-Enterprise, LLC, formerly Helena Chemical Company)+TX, Rhizopogon fulvigleba (e.g. Myco-Sol from Agri-Enterprise, LLC, formerly Helena Chemical Company)+TX, Talaromyces flavus strain V117b+TX, Trichoderma asperellum strain (Eco-T from Plant Health Products, ZA)+TX, Trichoderma asperellum strain kd (e.g. T-Gro from Andermatt Biocontrol)+TX, Trichoderma atroviride in particular strain no. V08/002387+TX, Trichoderma atroviride strain CNCM 1-1237 (e.g. Esquive® WP from Agrauxine, FR)+TX, Trichoderma atroviride strain LC52 (also known as Trichoderma atroviride strain LU132, e.g. Sentinel from Agrimm Technologies Limited)+TX, Trichoderma atroviride strain no. NMI No. V08/002388+TX, Trichoderma atroviride strain no. NMI No. V08/002389+TX, Trichoderma atroviride strain no. NMI No. V08/002390+TX, Trichoderma atroviride strain SC1 (described in WO2009/116106)+TX, Trichoderma harzianum strain 1295-22+TX, Trichoderma harzianum strain ITEM 908+TX, Trichoderma harzianum strain T-22 (e.g. Trianum-P from Andermatt Biocontrol or Koppert)+TX, Trichoderma harzianum strain TSTh20, +TX, Trichoderma virens strain GI-3+TX, Trichoderma virens strain GL-21 (e.g. SoilGard® from Certis, USA)+TX, Trichoderma viride strain B35 (Pietr et al., 1993, Zesz. Nauk. A R w Szczecinie 161: 125-137)+TX, Verticillium albo-atrum (formerly V. dahliae) strain WCS850 (CBS 276.92, e.g. Dutch Trig from Tree Care Innovations)+TX; Agrobacterium radiobacter strain K84 (Galltrol from AgBiochem Inc.), +TX, Bacillus amyloliquefaciens in particular strain PTS-4838 (e.g. AVEO from Valent Biosciences, US), +TX, Bacillus mycoides, isolate J. (e.g. BmJ from Certis USA LLC), +TX, Bacillus sphaericus in particular Serotype H5a5b strain 2362 (strain ABTS-1743) (e.g. VECTOLEX® from Valent BioSciences, US), +TX, Bacillus thuringiensis israelensis strain BMP 144 (e.g. AQUABAC® by Becker Microbial Products IL)+TX, Bacillus thuringiensis subsp. aizawai strain GC-91+TX, Bacillus thuringiensis subsp. aizawai, in particular serotype H-7 (e.g. FLORBAC® WG from Valent BioSciences, US)+TX, Bacillus thuringiensis subsp. aizawai, in particular strain ABTS-1857 (SD-1372, e.g. XENTARI® from Valent BioSciences)+TX, Bacillus thuringiensis subsp. israelensis (serotype H-14) strain AM65-52 (Accession No. ATCC 1276) (e.g. VECTOBAC® by Valent BioSciences, US)+TX, Bacillus thuringiensis subsp. kurstaki strain ABTS 351+TX, Bacillus thuringiensis subsp. kurstaki strain BMP 123 (from Becker Microbial Products, IL, BARITONE from Bayer CropScience)+TX, Bacillus thuringiensis subsp. kurstaki strain EG 2348 (LEPINOX from Certis, US)+TX, Bacillus thuringiensis subsp. kurstaki strain EG 7841 (CRYMAX from Certis, US)+TX, Bacillus thuringiensis subsp. kurstaki strain HD-1 (e.g. DIPEL® ES from Valent BioSciences, US)+TX, Bacillus thuringiensis subsp. kurstaki strain PB 54+TX, Bacillus thuringiensis subsp. kurstaki strain SA 11 (JAVELIN from Certis, US)+TX, Bacillus thuringiensis subsp. kurstaki strain SA 12 (THURICIDE from Certis, US)+TX, Bacillus thuringiensis subsp. tenebrionis strain NB 176 (SD-5428, e.g. NOVODOR® FC from BioFa DE)+TX, Bacillus thuringiensis var. Colmeri (e.g. TIANBAOBTC by Changzhou Jianghai Chemical Factory)+TX, Bacillus thuringiensis var. japonensis strain Buibui+TX, Bacillus thuringiensis var. kurstaki strain EVB-113-19 (e.g., BIOPROTEC® from AEF Global)+TX, Brevibacillus laterosporus+TX, Burkholderia spp. in particular Burkholderia rinojensis strain A396 (also known as Burkholderia rinojensis strain MBI 305) (Accession No. NRRL B-50319, WO 2011/106491 and WO 2013/032693, e.g. MBI206 TGAI and ZELTO® from Marrone Bio Innovations), +TX, Chromobacterium subtsugae in particular strain PRAA4-1T (e.g. MBI-203, e.g. GRANDEVO® from Marrone Bio Innovations)+TX, Lecanicillium muscarium Ve6 (MYCOTAL from Koppert)+TX, Paenibacillus popilliae (formerly Bacillus popilliae, e.g. MILKY SPORE POWDER™ or MILKY SPORE GRANULAR™ from St. Gabriel Laboratories)+TX, Serratia entomophila (e.g. INVADE® by Wrightson Seeds)+TX, Serratia marcescens in particular strain SRM (Accession No. MTCC 8708)+TX, Trichoderma asperellum (TRICHODERMAX from Novozymes)+TX, Wolbachia pipientis ZAP strain (e.g., ZAP MALES® from MosquitoMate)+TX;
Beauveria bassiana strain ATCC 74040 (e.g. NATURALIS® from Intrachem Bio Italia)+TX, Beauveria bassiana strain ATPO2 (Accession No. DSM 24665), Apopka 97 (PREFERAL from SePRO)+TX, Beauveria bassiana strain GHA (Accession No. ATCC74250, e.g. BOTANIGUARD® ES and MYCONTROL-O® from Laverlam International Corporation)+TX, Metarhizium anisopliae 3213-1 (deposited under NRRL accession number 67074 disclosed in WO 2017/066094, Pioneer Hi-Bred International)+TX, Metarhizium robertsii 15013-1 (deposited under NRRL accession number 67073)+TX, Metarhizium robertsii 23013-3 (deposited under NRRL accession number 67075)+TX, Paecilomyces lilacinus strain 251 (MELOCON from Certis, US)+TX; Cydia pomonella (codling moth) granulosis virus (GV)+TX, Helicoverpa armigera (cotton bollworm) nuclear polyhedrosis virus (NPV)+TX, of Adoxophyes orana (summer fruit tortrix) granulosis virus (GV)+TX, Spodoptera exigua (beet armyworm) mNPV+TX, Spodoptera frugiperda (fall armyworm) mNPV+TX;
Burkholderia spp. in particular Burkholderia cepacia (formerly known as Pseudomonas cepacia)+TX, Gigaspora spp.+TX, Glomus spp.+TX, Laccaria spp.+TX, LactoBacillus buchneri+TX, Paraglomus spp.+TX, Pisolithus tinctorus+TX, Pseudomonas spp.+TX, Rhizobium spp. in particular Rhizobium trifolii+TX, Rhizopogon spp.+TX, Scleroderma spp.+TX, Streptomyces spp.+TX, Suillus spp.+TX, Agrobacterium spp.+TX, Azorhizobium caulinodans+TX, Azospirillum spp.+TX, Azotobacter spp.+TX, Bradyrhizobium spp.+TX, Gigaspora monosporum+TX; Allium sativum (NEMGUARD from Eco-Spray, BRALIC from ADAMA)+TX, Armour-Zen+TX, Artemisia absinthium+TX, Biokeeper WP+TX, Brassicaceae extract in particular oilseed rape powder or mustard powder+TX, Cassia nigricans+TX, Celastrus angulatus+TX, Chenopodium anthelminticum+TX, Chenopodium quinoa saponin extract from quinoa seeds (e.g. Heads Up® (Saponins of Quinoa) from Heads Up plant Protectants, CA)+TX, Chitin+TX, Dryopteris filix-mas+TX, Equisetum arvense+TX, Fortune Aza+TX, Fungastop+TX, Melaleuca alternifolia extract (TIMOREX GOLD from STK)+TX, naturally occurring Blad polypeptide extracted from Lupin seeds (FRACTURE® from FMC)+TX, naturally occurring Blad polypeptide extracted from Lupin seeds (PROBLAD® from Certis EU)+TX, Pyrethrins+TX, Quassia amara+TX, Quercus+TX, Quillaja extract (QL AGRI 35 from BASF)+TX, REGALIA MAXX from Marrone Bio)+TX, Requiem™ Insecticide+TX, Reynoutria sachalinensis extract (REGALLIA+TX, ryania/ryanodine+TX, Symphytum officinale+TX, Tanacetum vulgare+TX, Thymol+TX, Thymol mixed with Geraniol (CEDROZ from Eden Research)+TX, Thymol mixed with Geraniol and Eugenol (MEVALONE from Eden Research)+TX, Triact 70+TX, TriCon+TX, Tropaeulum majus+TX, Urtica dioica+TX, Veratrin+TX, Viscum album+TX;
mercuric oxide+TX, octhilinone+TX, thiophanate-methyl+TX;
MGK 264+TX, 2-(2-butoxyethoxy)ethyl piperonylate+TX, 2-isovalerylindan-1,3-dione+TX, 4-(quinoxalin-2-ylamino)benzenesulfonamide+TX, 5-(1,3-benzodioxol-5-yl)-3-hexylcyclohex-2-enone+TX, acibenzolar+TX, acibenzolar-S-methyl+TX, alpha-bromadiolone+TX, alpha-chlorohydrin+TX, aluminium phosphide+TX, anthraquinone+TX, antu+TX, arsenous oxide+TX, barium carbonate+TX, benoxacor+TX, bisthiosemi+TX, brodifacoum+TX, bromadiolone+TX, bromethalin+TX, calcium cyanide+TX, chloralose+TX, chlorophacinone+TX, cholecalciferol+TX, cloquintocet (including cloquintocet-mexyl)+TX, copper naphthenate+TX, copper oxychloride+TX, coumachlor+TX, coumafuryl+TX, coumatetralyl+TX, crimidine+TX, cyprosulfamide+TX, diazinon+TX, dichlormid+TX, dicyclopentadiene+TX, difenacoum+TX, difethialone+TX, diphacinone+TX, ergocalciferol+TX, farnesol+TX, farnesol with nerolidol+TX, fenchlorazole (including fenchlorazole-ethyl)+TX, fenclorim+TX, flocoumafen+TX, fluoroacetamide+TX, flupropadine+TX, flupropadine hydrochloride+TX, fluxofenim+TX, furilazole+TX, gamma-HCH+TX, guazatine+TX, guazatine acetates+TX, HCH+TX, hydrogen cyanide+TX, imanin+TX, iodomethane+TX, isoxadifen (including isoxadifen-ethyl)+TX, lindane+TX, magnesium phosphide+TX, MB-599+TX, mefenpyr (including mefenpyr-diethyl)+TX, metcamifen+TX, methiocarb+TX, methyl bromide+TX, nerolidol+TX, norbormide+TX, petroleum oils+TX, phosacetim+TX, phosphine+TX, phosphorus+TX, pindone+TX, piperonyl butoxide+TX, piprotal+TX, potassium arsenite+TX, probenazole+TX, propyl isomer+TX, pyridin-4-amine+TX, pyrinuron+TX, Reynoutria sachalinensis extract+TX, ribavirin+TX, S421+TX, scilliroside+TX, sesamex+TX, sesasmolin+TX, sodium arsenite+TX, sodium cyanide+TX, sodium fluoroacetate+TX, strychnine+TX, sulfoxide+TX, thallium sulfate+TX, thiram+TX, trimethacarb+TX, warfarin+TX, zinc naphthenate+TX, zinc phosphide+TX, ziram+TX.

In addition, the compositions of the invention may also be applied with one or more systemically acquired resistance inducers (“SAR” inducer). SAR inducers are known and described in, for example, U.S. Pat. No. 6,919,298 and include, for example, salicylates and the commercial SAR inducer acibenzolar-S-methyl.

The compounds of formula (I) as defined in the present invention are normally used in the form of compositions and can be applied to the crop area or plant to be treated, simultaneously or in succession with further compounds. These further compounds can be e.g. fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of formula (I) as defined in the present invention may be used in the form of (fungicidal) compositions for controlling or protecting against phytopathogenic microorganisms, comprising as active ingredient at least one compound of formula (I) as defined in the present invention or of at least one preferred individual compound as above-defined, in free form or in agrochemically usable salt form, and at least one of the above-mentioned adjuvants.

The invention therefore provides a composition, preferably a fungicidal composition, comprising at least one compound of formula (I) as defined in the present invention, an agriculturally acceptable carrier and optionally an adjuvant. An agricultural acceptable carrier is for example a carrier that is suitable for agricultural use. Agricultural carriers are well known in the art. Preferably said composition may comprise at least one or more pesticidally active compounds, for example an additional fungicidal active ingredient in addition to the compound of formula (I) as defined in the present invention.

A further aspect of invention is related to a method of controlling or preventing an infestation of plants, e.g. useful plants such as crop plants, propagation material thereof, e.g. seeds, harvested crops, e.g. harvested food crops, or of non-living materials by phytopathogenic or spoilage microorganisms or organisms potentially harmful to man, especially fungal organisms, which comprises the application of a compound of formula (I) as defined in the present invention or of a preferred individual compound as above-defined as active ingredient to the plants, to parts of the plants or to the locus thereof, to the propagation material thereof, or to any part of the non-living materials.

Controlling or preventing means reducing infestation by insects or by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, which comprises the application of a compound of formula (I) as defined in the present invention, or an agrochemical composition which contains at least one of said compounds, is foliar application. The frequency of application and the rate of application will depend on the risk of infestation by the corresponding pathogen or insect. However, the compounds of formula (I) as defined in the present invention can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula (I) as defined in any the present invention may also be applied to seeds (coating) by impregnating the seeds or tubers either with a liquid formulation of the fungicide or coating them with a solid formulation.

A formulation, e.g. a composition containing the compound of formula (I) as defined in the present invention, and, if desired, a solid or liquid adjuvant or monomers for encapsulating the compound of formula (I) as defined in the present invention, may be prepared in a known manner, typically by intimately mixing and/or grinding the compound with extenders, for example solvents, solid carriers and, optionally, surface active compounds (surfactants).

The application methods for the compositions, that is the methods of controlling pathogens of the abovementioned type, such as spraying, atomizing, dusting, brushing on, dressing, scattering or pouring—which are to be selected to suit the intended aims of the prevailing circumstances—and the use of the compositions for controlling pathogens of the abovementioned type are other subjects of the invention. Typical rates of concentration are between 0.1 and 1000 ppm, preferably between 0.1 and 500 ppm, of active ingredient. The rate of application per hectare is preferably 1 g to 2000 g of active ingredient per hectare, more preferably 10 to 1000 g/ha, most preferably 10 to 600 g/ha. When used as seed drenching agent, convenient dosages are from 10 mg to 1 g of active substance per kg of seeds.

When the combinations of the present invention are used for treating seed, rates of 0.001 to 50 g of a compound of formula (I) per kg of seed, preferably from 0.01 to 10 g per kg of seed are generally sufficient.

Suitably, a composition comprising a compound of formula (I) as defined in the present invention according to the present invention is applied either preventative, meaning prior to disease development or curative, meaning after disease development.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g. by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the ondensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g. as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compound of formula (I) as defined in the present invention together with component (B) and (C), and optionally other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

Whereas it is preferred to formulate commercial products as concentrates, the end user will normally use dilute formulations.

### EXAMPLES

The Examples which follow serve to illustrate the invention. Certain compounds of the invention can be distinguished from known compounds by virtue of greater efficacy at low application rates, which can be verified by the person skilled in the art using the experimental procedures outlined in the Examples.

### Formulation Examples

| Wettable powders | a) | b) | c) |
|---|---|---|---|
| active ingredient [compound of formula (I)] | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The active ingredient is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredient [compound of formula (I)] | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The active ingredient is thoroughly mixed with the adjuvants and the mixture is thoroughly ground in a suitable mill, affording powders that can be used directly for seed treatment.

### Emulsifiable Concentrate

| | |
|---|---|
| active ingredient [compound of formula (I)] | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredient [compound of formula (I)] | 5% | 6% | 4% |
| talcum | 95% | — | — |
| Kaolin | — | 94% | — |
| mineral filler | — | — | 96% |

Ready-for-use dusts are obtained by mixing the active ingredient with the carrier and grinding the mixture in a suitable mill. Such powders can also be used for dry dressings for seed.

### Extruder Granules

| | |
|---|---|
| Active ingredient [compound of formula (I)] | 15% |
| sodium lignosulfonate | 2% |
| carboxymethylcellulose | 1% |
| Kaolin | 82% |

The active ingredient is mixed and ground with the adjuvants, and the mixture is moistened with water. The mixture is extruded and then dried in a stream of air.

### Coated Granules

| | |
|---|---|
| Active ingredient [compound of formula (I)] | 8% |
| polyethylene glycol (mol. wt. 200) | 3% |
| Kaolin | 89% |

The finely ground active ingredient is uniformly applied, in a mixer, to the kaolin moistened with polyethylene glycol. Non-dusty coated granules are obtained in this manner.

### Suspension Concentrate

| | |
|---|---|
| active ingredient [compound of formula (I)] | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Flowable Concentrate for Seed Treatment

| | |
|---|---|
| active ingredient [compound of formula (I)] | 40% |
| propylene glycol | 5% |
| copolymer butanol PO/EO | 2% |
| tristyrenephenole with 10-20 moles EO | 2% |
| 1,2-benzisothiazolin-3-one (in the form of a 20% solution in | 0.5% |
| water) | |
| monoazo-pigment calcium salt | 5% |
| Silicone oil (in the form of a 75% emulsion in water) | 0.2% |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of a combination of the compound of formula (I) are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### Analytical Methods:

Throughout this description, temperatures are given in degrees Celsius (° C.) and “mp.” means melting point. LC/MS means Liquid Chromatography Mass Spectrometry and the description of the apparatus and the method is as follows:

### Method A:

Spectra were recorded on a Mass Spectrometer from Waters Corporation (SQD, SQDII or QDA Single quadrupole mass spectrometer) equipped with an electrospray source (Polarity: positive and negative ions), Capillary: 0.8-3.00 kV, Cone: 5-30 V, Source Temperature: 120-150° C., Desolvation Temperature: 350-600° C., Cone Gas Flow: 50-150 l/h, Desolvation Gas Flow: 650-1000 l/h, Mass range: 110 to 950 Da and an Acquity UPLC from Waters Corporation: Binary pump, heated column compartment, diode-array detector and ELSD. Column: Waters UPLC HSS T3, 1.8 μm, 30×2.1 mm, Temp: 60° C., DAD Wavelength range (nm): 210 to 400, Runtime: 1.5 min; Solvents: A=water+5% MeOH+0.05% HCOOH, B=Acetonitrile+0.05% HCOOH; Flow (mL/min) 0.85, Gradient: 10% B isocratic for 0.2 min, then 10-100% B in 1.0 min, 100% B isocratic for 0.2 min, 100-10% B in 0.05 min, 10% B isocratic for 0.05 min.

### Method B:

Spectra were recorded on a ACQUITY Mass Spectrometer from Waters Corporations (SQD or SQDII Single quadrupole mass spectrometer) equipped with an electrospray source (Polarity: positive or negative ions, Capillary: 3.0 kV, Cone: 30V, Extractor: 3.00 V, Source Temperature: 150° C., Desolvation Temperature: 400° C., Cone Gas Flow: 60 L/hr, Desolvation Gas Flow: 700 L/hr, Mass range: 140 to 800 Da) and an ACQUITY UPLC from Waters Corporations with solvent degasser, binary pump, heated column compartment and diode-array detector. Column: Waters UPLC HSS T3, 1.8 μm, 30×2.1 mm, Temp: 60° C., DAD Wavelength range (nm): 210 to 400, Solvent Gradient: A=Water/Methanol 9:1+0.1% formic acid, B=Acetonitrile+0.1% formic acid, gradient: 0-100% B in 2.5 min; Flow (mL/min) 0.75.

### Method C:

Spectra were recorded on a on a Mass Spectrometer from Agilent (Single quad mass spectrometer) equipped with Electron Spray (Polarity: positive and negative ions), Capillary: 4.00 kV, Charging Voltage, 2.00 kV, Nitrogen Gas Flow:12.0 L/min, Nebulizer Pressure: 40 psig, Mass range: 100 to 1000 m/z, dry gas temperature 250° C., Vaporizer temperature 200° C. and an UPLC from Waters: quaternary pump, heated column compartment, Variable wave length detector. Column: Kinetex Evo_50 mm, 1.7 μm (2.1*50 mm), column Temp: Ambient, Wavelength (nm): 200-400 nm, Gradient: A=acetonitrile, B=10 mM ammonium formate in water. Gradient time/% A: 0/2, 0.2/2, 1.5/45, 2.4/95, 3.3/2, 3.5/2; Flow rate: 0.5 mL/min.

The below Table A gathers for compounds of formula (I):
LC-MS data, such as retention time (RT), [M+H]⁺,
the type of method, and/or
melting point (mp).

TABLE A

| | | | | [M + | | |
|---|---|---|---|---|---|---|
| | | | | H] | | |
| | | | RT | (meas- | Meth- | mp |
| | Compound name | Structure | (min) | ured) | od | (° C.) |
| 1 | methyl N-[5-[5-[(4-fluoro-3- methoxy-phenyl)- (methoxymethyl) carbamoyl ]pyrazolo[1,5- a]pyridin-3- yl]-2-pyridyl] carbamate | 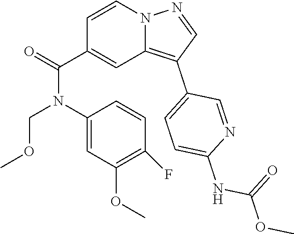 | 1.84 | 480 | C | 145- 150 |
| 2 | methyl N-[5-[5-[(4-chloro- 3-methoxy-phenyl)- (cyanomethyl) carbamoyl] pyrazolo[1,5- a]pyridin-3-yl]- 2-pyridyl]carbamate | 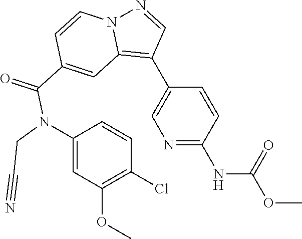 [SMILES: COC(=O)Nc1ccc(-c2cnn3ccc(C(=O)N(CC#N)c4ccc(Cl)c(OC)c4)cc23)cn1] | 0.91 | 491 | A | 227- 230 |
| 3 | methyl N-[5-[5- [cyanomethyl- (4-fluoro-3- methoxy- phenyl)carbamoyl] pyrazolo [1,5-a]pyridin-3-yl]-2- pyridyl]carbamate | 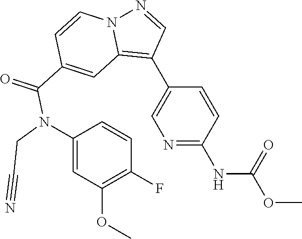 [SMILES: COC(=O)Nc1ccc(-c2cnn3ccc(C(=O)N(CC#N)c4ccc(F)c(OC)c4)cc23)cn1] | 0.87 | 475 | A | 194- 195 |
| 4 | methyl N-[5-[5-[(4-fluoro-3- methoxy-phenyl)- methyl- carbamoyl] pyrazolo[1,5- a]pyridin-3-yl]-2- pyridyl]carbamate | 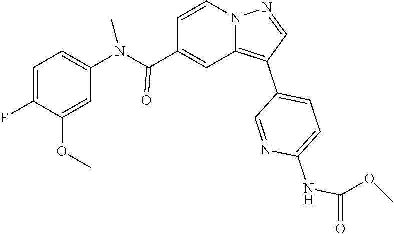 [SMILES: COC(=O)Nc1ccc(-c2cnn3ccc(C(=O)N(C)c4ccc(F)c(OC)c4)cc23)cn1] | 0.87 | 450 | A | |
| 5 | methyl N-[4-[5-[(4- chlorophenyl)-methyl- carbamoyl]pyrazolo[1,5- a]pyridin-3- yl]phenyl]carbamate | 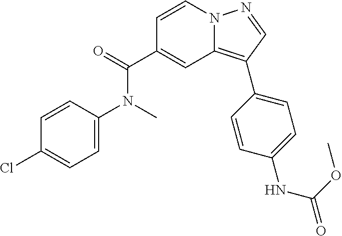 [SMILES: COC(=O)Nc1ccc(-c2cnn3ccc(C(=O)N(C)c4ccc(Cl)cc4)cc23)cc1] | 0.97 | 435 | A | |
| 6 | 3-(4- acetamidophenyl)-N- (4-chlorophenyl)- N-methyl- pyrazolo[1,5- a]pyridine-5- carboxamide | 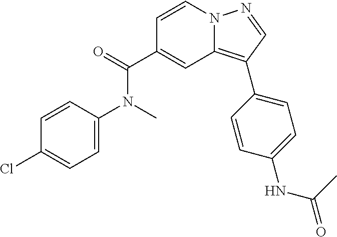 [SMILES: CC(=O)Nc1ccc(-c2cnn3ccc(C(=O)N(C)c4ccc(Cl)cc4)cc23)cc1] | 0.89 | 419 | A | |
| 7 | methyl N-[5-[5-[(6- methoxy-3- pyridyl)-methyl- carbamoyl]pyrazolo[1,5- a]pyridin-3-yl]-2- pyridyl]carbamate | 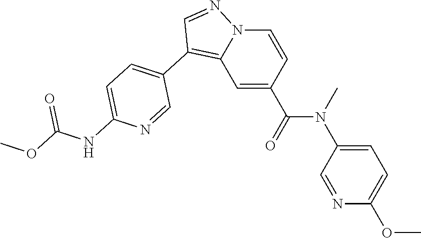 | 1.16 | 433 | B | |
| 8 | 3-(6-acetamido- 3-pyridyl)- N-(6-methoxy- 3-pyridyl)-N- methyl-pyrazolo[1,5- a]pyridine-5- carboxamide | 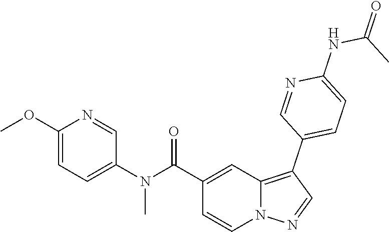 [SMILES: COc1ccc(N(C)C(=O)c2ccn3ncc(-c4ccc(NC(C)=O)nc4)c3c2)cn1] | 1.03 | 417 | B | |
| 9 | 3-(4- acetamidophenyl)-N- (6-methoxy- 3-pyridyl)-N- methyl-pyrazolo[1,5- a]pyridine-5- carboxamide | 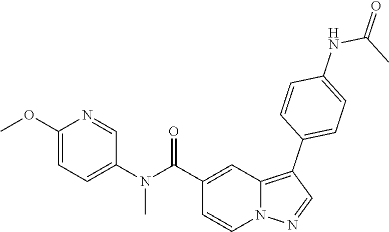 [SMILES: COc1ccc(N(C)C(=O)c2ccn3ncc(-c4ccc(NC(C)=O)cc4)c3c2)cn1] | 1.11 | 416 | B | |
| 10 | 3-[4-[(2- methoxyacetyl)amino] phenyl]-N- (6-methoxy-3- pyridyl)-N-methyl- pyrazolo[1,5- a]pyridine-5- carboxamide | 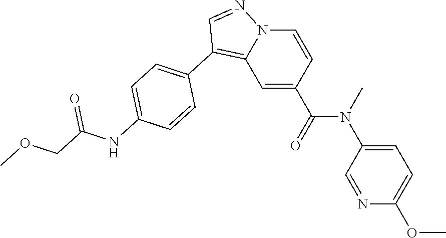 | 1.19 | 446 | B | |
| 11 | 3-[6- (ethylcarba- moylamino)-3- pyridyl]-N- (6-methoxy-3- pyridyl)-N-methyl- pyrazolo[1,5- a]pyridine-5- carboxamide | 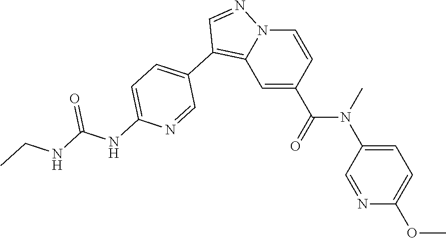 | 1.12 | 446 | B | |
| 12 | methyl N-[5-[5-[(4- cyanophenyl)-methyl- carbamoyl]pyrazolo[1,5- a]pyridin-3-yl]-2- pyridyl]carbamate | 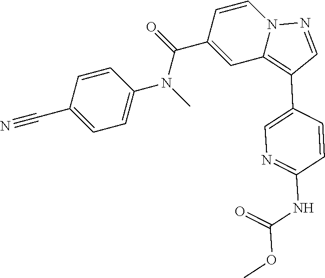 [SMILES: COC(=O)Nc1ccc(-c2cnn3ccc(C(=O)N(C)c4ccc(C#N)cc4)cc23)cn1] | 1.17 | 427 | B | |
| 13 | 3-(6-acetamido- 3-pyridyl)- N-(4-cyanophenyl)-N- methyl-pyrazolo[1,5- a]pyridine-5- carboxamide | 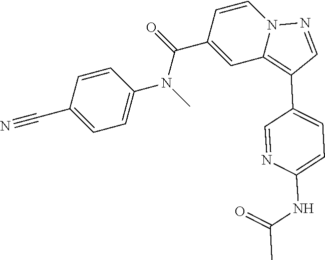 [SMILES: CC(=O)Nc1ccc(-c2cnn3ccc(C(=O)N(C)c4ccc(C#N)cc4)cc23)cn1] | 1.04 | 411 | B | |
| 14 | 3-(4- acetamidophenyl)-N- (4-cyanophenyl)- N-methyl- pyrazolo[1,5- a]pyridine-5- carboxamide | 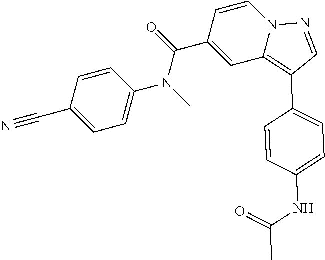 [SMILES: CC(=O)Nc1ccc(-c2cnn3ccc(C(=O)N(C)c4ccc(C#N)cc4)cc23)cc1] | 1.13 | 410 | B | |
| 15 | N-(4- cyanophenyl)-3-[4- [(2-methoxyacetyl) amino]phenyl]- N-methyl-pyrazolo[1,5- a]pyridine-5- carboxamide | 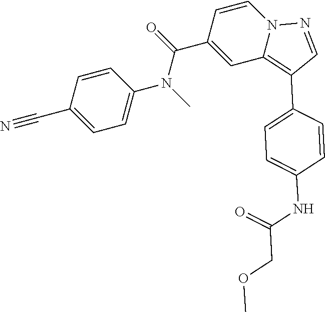 [SMILES: COCC(=O)Nc1ccc(-c2cnn3ccc(C(=O)N(C)c4ccc(C#N)cc4)cc23)cc1] | 1.21 | 440 | B | |
| 16 | N-(4- cyanophenyl)-3-[6- (ethylcarba- moylamino)-3- pyridyl]-N-methyl- pyrazolo[1,5- a]pyridine-5- carboxamide | 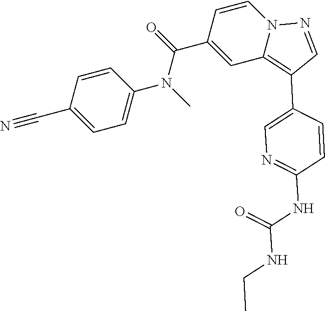 [SMILES: CCNC(=O)Nc1ccc(-c2cnn3ccc(C(=O)N(C)c4ccc(C#N)cc4)cc23)cn1] | 1.14 | 440 | B | |
| 17 | methyl N-[5-[5-[(2- methoxy-4- pyridyl)-methyl- carbamoyl]pyrazolo[1,5- a]pyridin-3-yl]-2- pyridyl]carbamate | 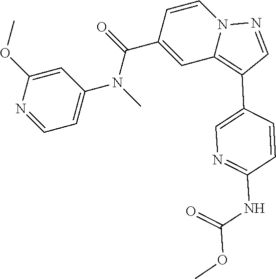 [SMILES: COC(=O)Nc1ccc(-c2cnn3ccc(C(=O)N(C)c4ccnc(OC)c4)cc23)cn1] | 1.16 | 433 | B | |
| 18 | 3-(6-acetamido- 3-pyridyl)- N-(2-methoxy- 4-pyridyl)-N- methyl-pyrazolo[1,5- a]pyridine-5- carboxamide | 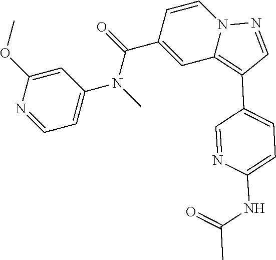 [SMILES: COc1cc(N(C)C(=O)c2ccn3ncc(-c4ccc(NC(C)=O)nc4)c3c2)ccn1] | 1.03 | 417 | B | |
| 19 | 3-(4- acetamidophenyl)-N- (2-methoxy- 4-pyridyl)-N- methyl-pyrazolo[1,5- a]pyridine-5- carboxamide | 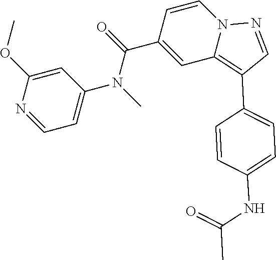 [SMILES: COc1cc(N(C)C(=O)c2ccn3ncc(-c4ccc(NC(C)=O)cc4)c3c2)ccn1] | 1.12 | 416 | B | |
| 20 | 3-[4-[(2- methoxyacetyl)amino] phenyl]-N- (2-methoxy-4- pyridyl)-N-methyl- pyrazolo[1,5- a]pyridine-5- carboxamide | 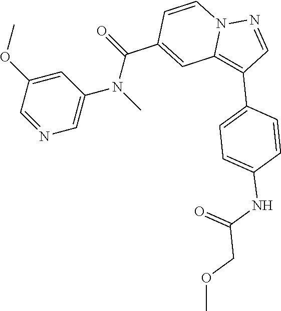 [SMILES: COCC(=O)Nc1ccc(-c2cnn3ccc(C(=O)N(C)c4cncc(OC)c4)cc23)cc1] | 1.2 | 446 | B | |
| 21 | 3-[6- (ethylcarba- moylamino)-3- pyridyl]-N-(2- methoxy-4- pyridyl)-N-methyl- pyrazolo[1,5- a]pyridine-5- carboxamide | 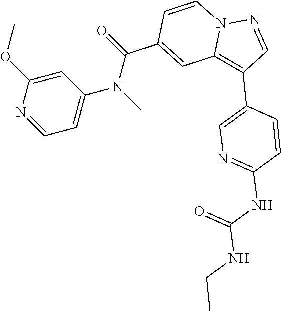 [SMILES: CCNC(=O)Nc1ccc(-c2cnn3ccc(C(=O)N(C)c4ccnc(OC)c4)cc23)cn1] | 1.13 | 446 | B | |
| 22 | methyl N-[5-[5-[(3- fluorophenyl)-methyl- carbamoyl] pyrazolo[1,5- a]pyridin-3-yl]-2- pyridyl]carbamate | 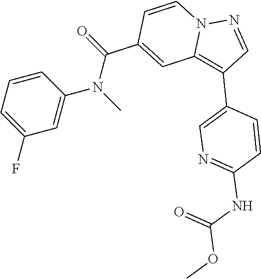 [SMILES: COC(=O)Nc1ccc(-c2cnn3ccc(C(=O)N(C)c4cccc(F)c4)cc23)cn1] | 1.29 | 420 | B | |
| 23 | 3-(6-acetamido- 3-pyridyl)- N-(3-fluorophenyl)-N- methyl-pyrazolo[1,5- a]pyridine-5- carboxamide | 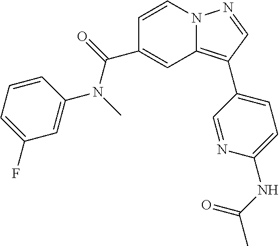 [SMILES: CC(=O)Nc1ccc(-c2cnn3ccc(C(=O)N(C)c4cccc(F)c4)cc23)cn1] | 1.15 | 404 | B | |
| 24 | 3-(4- acetamidophenyl)-N- (3-fluorophenyl)- N-methyl- pyrazolo[1,5- a]pyridine-5- carboxamide | 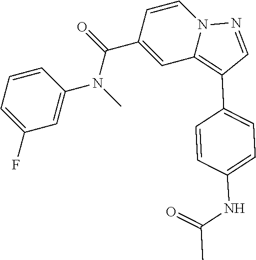 [SMILES: CC(=O)Nc1ccc(-c2cnn3ccc(C(=O)N(C)c4cccc(F)c4)cc23)cc1] | 1.23 | 403 | B | |
| 25 | N-(3-fluorophenyl)- 3-[4-[(2- methoxyacetyl)amino] phenyl]-N- methyl-pyrazolo[1,5- a]pyridine-5- carboxamide | 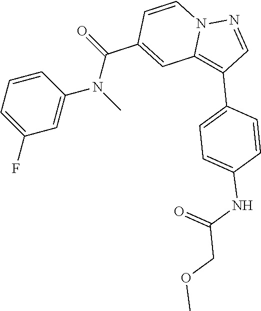 [SMILES: COCC(=O)Nc1ccc(-c2cnn3ccc(C(=O)N(C)c4cccc(F)c4)cc23)cc1] | 1.32 | 433 | B | |
| 26 | 3-[6- (ethylcarba- moylamino)-3- pyridyl]-N-(3- fluorophenyl)- N-methyl-pyrazolo[1,5- a]pyridine-5- carboxamide | 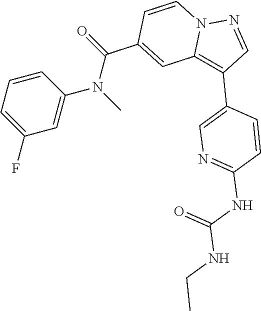 [SMILES: CCNC(=O)Nc1ccc(-c2cnn3ccc(C(=O)N(C)c4cccc(F)c4)cc23)cn1] | 1.25 | 433 | B | |
| 27 | methyl N-[5-[5-[(4- methoxy-2- pyridyl)-methyl- carbamoyl] pyrazolo[1,5- a]pyridin-3-yl]-2- pyridyl]carbamate | 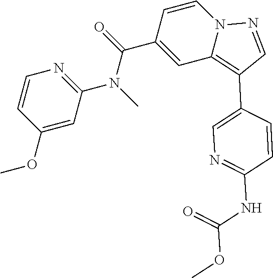 [SMILES: COC(=O)Nc1ccc(-c2cnn3ccc(C(=O)N(C)c4cc(OC)ccn4)cc23)cn1] | 1.08 | 433 | B | |
| 28 | 3-(6-acetamido- 3-pyridyl)- N-(4-methoxy- 2-pyridyl)-N- methyl-pyrazolo[1,5- a]pyridine-5- carboxamide | 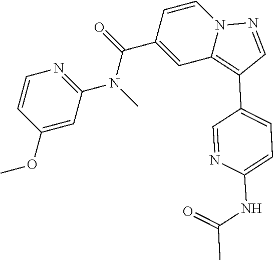 [SMILES: COc1ccnc(N(C)C(=O)c2ccn3ncc(-c4ccc(NC(C)=O)nc4)c3c2)c1] | 0.95 | 417 | B | |
| 29 | 3-(4- acetamidophenyl)-N- (4-methoxy- 2-pyridyl)-N- methyl-pyrazolo[1,5- a]pyridine-5- carboxamide | 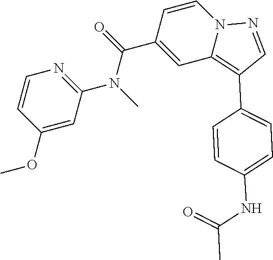 [SMILES: COc1ccnc(N(C)C(=O)c2ccn3ncc(-c4ccc(NC(C)=O)cc4)c3c2)c1] | 1.03 | 416 | B | |
| 30 | 3-[4-[(2- methoxyacetyl)amino] phenyl]-N- (4-methoxy-2- pyridyl)-N-methyl- pyrazolo[1,5- a]pyridine-5- carboxamide | 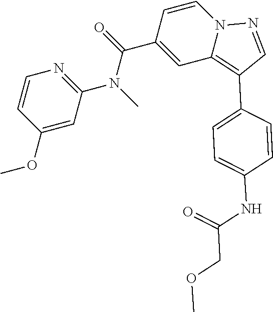 [SMILES: COCC(=O)Nc1ccc(-c2cnn3ccc(C(=O)N(C)c4cc(OC)ccn4)cc23)cc1] | 1.11 | 446 | B | |
| 31 | 3-[6- (ethylcarba- moylamino)-3- pyridyl]-N-(4-methoxy- 2-pyridyl)-N-methyl- pyrazolo[1,5- a]pyridine-5- carboxamide | 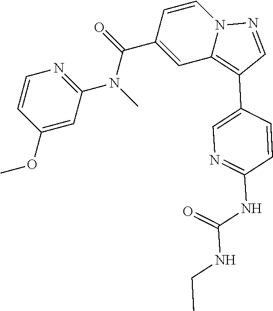 [SMILES: CCNC(=O)Nc1ccc(-c2cnn3ccc(C(=O)N(C)c4cc(OC)ccn4)cc23)cn1] | 1.05 | 446 | B | |
| 32 | methyl N-[5-[5-[(6-cyano-3- pyridyl)-methyl- carbamoyl]pyrazolo[1,5- a]pyridin-3-yl]-2- pyridyl]carbamate | 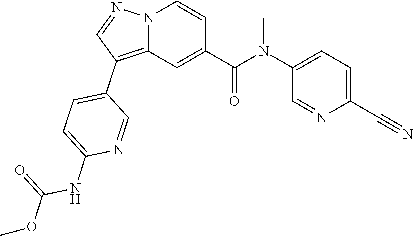 [SMILES: COC(=O)Nc1ccc(-c2cnn3ccc(C(=O)N(C)c4ccc(C#N)nc4)cc23)cn1] | 1.07 | 428 | B | |
| 33 | 3-(6-acetamido- 3-pyridyl)- N-(6-cyano-3- pyridyl)-N- methyl-pyrazolo[1,5- a]pyridine-5- carboxamide | 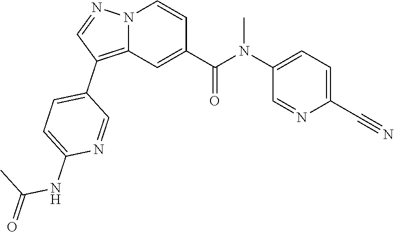 [SMILES: CC(=O)Nc1ccc(-c2cnn3ccc(C(=O)N(C)c4ccc(C#N)nc4)cc23)cn1] | 0.94 | 412 | B | |
| 34 | 3-(4- acetamidophenyl)-N- (6-cyano-3-pyridyl)-N- methyl-pyrazolo[1,5- a]pyridine-5- carboxamide | 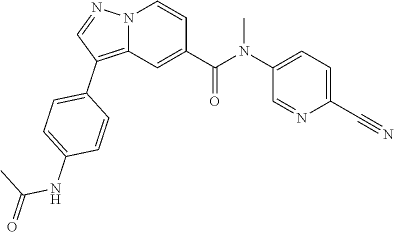 [SMILES: CC(=O)Nc1ccc(-c2cnn3ccc(C(=O)N(C)c4ccc(C#N)nc4)cc23)cc1] | 1.04 | 411 | B | |
| 35 | N-(6-cyano-3- pyridyl)-3-[4- [(2-methoxyacetyl) amino]phenyl]- N-methyl-pyrazolo[1,5- a]pyridine-5- carboxamide | 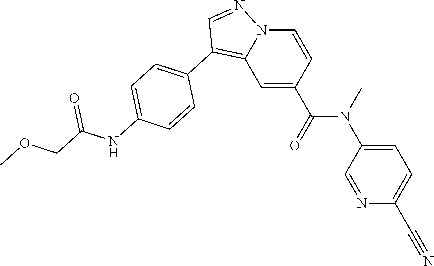 [SMILES: COCC(=O)Nc1ccc(-c2cnn3ccc(C(=O)N(C)c4ccc(C#N)nc4)cc23)cc1] | 1.12 | 441 | B | |
| 36 | N-(6-cyano- 3-pyridyl)-3-[6- (ethylcarba- moylamino)-3- pyridyl]-N-methyl- pyrazolo[1,5- a]pyridine-5- carboxamide | 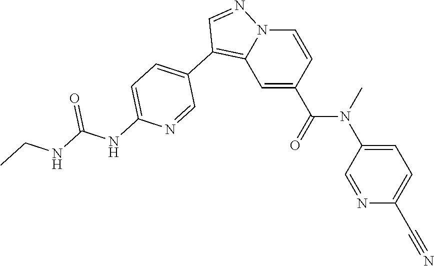 [SMILES: CCNC(=O)Nc1ccc(-c2cnn3ccc(C(=O)N(C)c4ccc(C#N)nc4)cc23)cn1] | 1.05 | 441 | B | |
| 37 | methyl N-[5-[5-[(6-chloro- 3-pyridyl)-methyl- carbamoyl] pyrazolo[1,5- a]pyridin-3-yl]-2- pyridyl]carbamate | 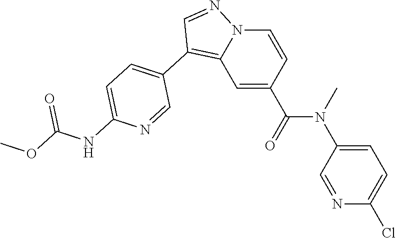 | 1.16 | 437 | B | |
| 38 | 3-(4- acetamidophenyl)-N- (6-chloro-3-pyridyl)-N- methyl-pyrazolo[1,5- a]pyridine-5- carboxamide | 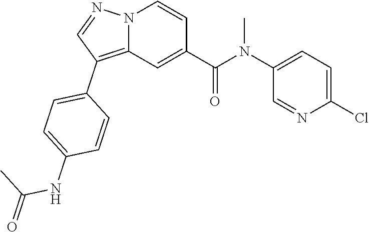 [SMILES: CC(=O)Nc1ccc(-c2cnn3ccc(C(=O)N(C)c4ccc(Cl)nc4)cc23)cc1] | 1.12 | 420 | B | |
| 39 | N-(6-chloro- 3-pyridyl)-3-[4- [(2-methoxyacetyl) amino]phenyl]- N-methyl-pyrazolo[1,5- a]pyridine-5- carboxamide | 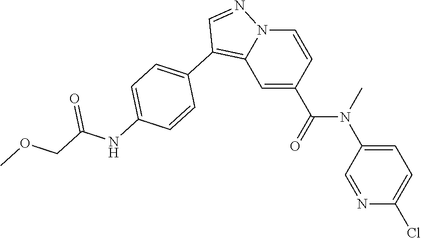 [SMILES: COCC(=O)Nc1ccc(-c2cnn3ccc(C(=O)N(C)c4ccc(Cl)nc4)cc23)cc1] | 1.2 | 450 | B | |
| 40 | N-(6-chloro- 3-pyridyl)-3-[6- (ethylcarba- moylamino)-3- pyridyl]-N-methyl- pyrazolo[1,5- a]pyridine-5- carboxamide | 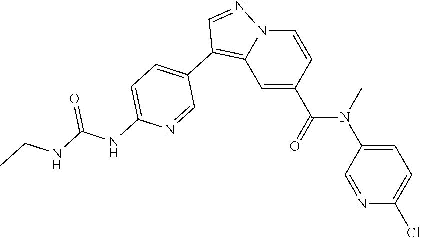 [SMILES: CCNC(=O)Nc1ccc(-c2cnn3ccc(C(=O)N(C)c4ccc(Cl)nc4)cc23)cn1] | 1.13 | 450 | B | |
| 41 | 3-(6-acetamido- 3-pyridyl)- N-(4-cyano-3-methoxy- phenyl)-N-methyl- pyrazolo[1,5- a]pyridine-5- carboxamide | 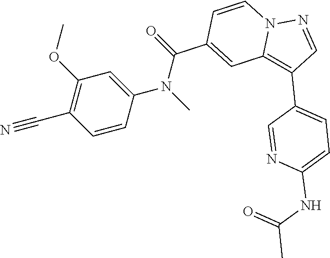 [SMILES: COc1cc(N(C)C(=O)c2ccn3ncc(-c4ccc(NC(C)=O)nc4)c3c2)ccc1C#N] | 1.16 | 441 | B | |
| 42 | methyl N-[5-[5-[(4-cyano-3- methyl-phenyl)-methyl- carbamoyl] pyrazolo[1,5- a]pyridin-3-yl]-2- pyridyl]carbamate | 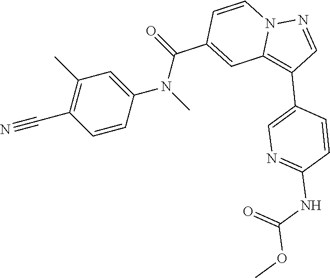 [SMILES: COC(=O)Nc1ccc(-c2cnn3ccc(C(=O)N(C)c4ccc(C#N)c(C)c4)cc23)cn1] | 1.25 | 441 | B | |
| 43 | 3-(6-acetamido- 3-pyridyl)- N-(4-cyano-3-methyl- phenyl)-N-methyl- pyrazolo[1,5- a]pyridine-5- carboxamide | 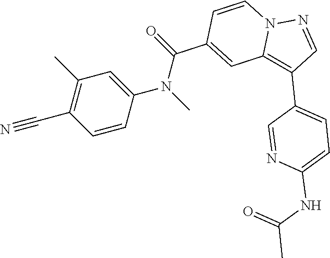 [SMILES: CC(=O)Nc1ccc(-c2cnn3ccc(C(=O)N(C)c4ccc(C#N)c(C)c4)cc23)cn1] | 1.13 | 425 | B | |
| 44 | 3-(4- acetamidophenyl)-N- (4-cyano-3- methyl-phenyl)- N-methyl-pyrazolo[1,5- a]pyridine-5- carboxamide | 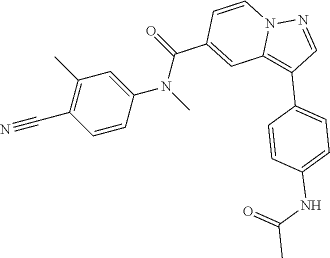 [SMILES: CC(=O)Nc1ccc(-c2cnn3ccc(C(=O)N(C)c4ccc(C#N)c(C)c4)cc23)cc1] | 1.21 | 424 | B | |
| 45 | N-(4-cyano-3-methyl- phenyl)-3-[4-[(2- methoxyacetyl)amino] phenyl]-N- methyl-pyrazolo[1,5- a]pyridine-5- carboxamide | 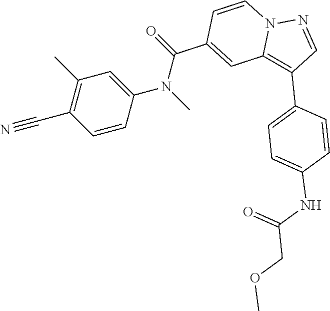 [SMILES: COCC(=O)Nc1ccc(-c2cnn3ccc(C(=O)N(C)c4ccc(C#N)c(C)c4)cc23)cc1] | 1.29 | 454 | B | |
| 46 | N-(4-cyano-3-methyl- phenyl)-3-[6- (ethylcarba- moylamino)-3- pyridyl]-N-methyl- pyrazolo[1,5- a]pyridine-5- carboxamide | 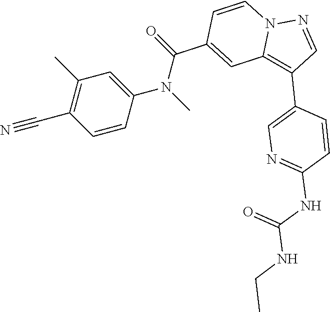 [SMILES: CCNC(=O)Nc1ccc(-c2cnn3ccc(C(=O)N(C)c4ccc(C#N)c(C)c4)cc23)cn1] | 1.22 | 454 | B | |
| 47 | 3-(6-acetamido- 3-pyridyl)- N-(4-fluoro-3-methoxy- phenyl)-N-methyl- pyrazolo[1,5- a]pyridine-5- carboxamide | 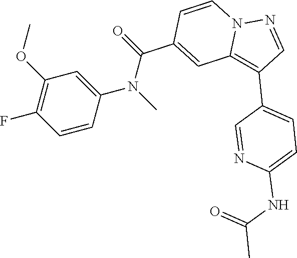 [SMILES: COc1cc(N(C)C(=O)c2ccn3ncc(-c4ccc(NC(C)=O)nc4)c3c2)ccc1F] | 1.16 | 434 | B | |

### Example 1: Preparation of methyl N-[5-[5-[(4-fluoro-3-methoxy-phenyl)-(methoxymethyl)carbamoyl]pyrazolol[1,5-a]pyridin-3-yl]-2-pyridyl]carbamate (Compound 1)

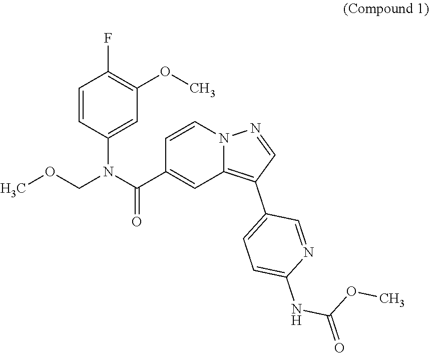

### Step A: Preparation of N-(4-fluoro-3-methoxy-phenyl)pyrazolo[1,5-a]pyridine-5-carboxamide

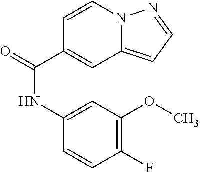 [SMILES: COc1cc(NC(=O)c2ccn3nccc3c2)ccc1F]

To an ice-cooled solution of pyrazolo[1,5-a]pyridine-5-carboxylic acid (CAS 104468-87-3, 1.40 g, 8.20 mmol) and 4-fluoro-3-methoxy-aniline (1.74 g, 12.3 mmol, 1.50 equiv.) in dimethylformamide (15.0 mL) was added triethylamine (3.43 mL, 24.6 mmol, 3.00 equiv.), followed by 1-[bis(dimethylamino)methylene]-1H-1,2,3-triazolo[4,5-b]pyridinium 3-oxide hexafluorophosphate (4.68 g, 12.3 mmol, 1.50 equiv.). The reaction mixture was stirred at room temperature for 16 hours. The reaction mixture was poured into water and extracted with ethyl acetate. The combined organic layers were dried over sodium sulfate, filtered, and concentrated under reduced pressure. The crude residue was purified over a silica gel cartridge (cyclohexane/ethyl acetate) to afford N-(4-fluoro-3-methoxy-phenyl)pyrazolo[1,5-a]pyridine-5-carboxamide as off white fluffy solid. ¹H-NMR (400 MHz, DMSO-d⁶, ppm) δ=3.84 (s, 3H), 6.88 (dd, 1H), 7.19-7.22 (m, 1H), 7.31-7.40 (m, 2H), 7.62-7.71 (dd, 1H), 8.13 (s, 1H), 8.39 (s, 1H), 8.80-8.83 (m, 1H), 10.45 (s, 1H)

### Step B: Preparation of N-(4-fluoro-3-methoxy-phenyl)-N-(methoxymethyl)pyrazolo[1,5-a]pyridine-5-carboxamide

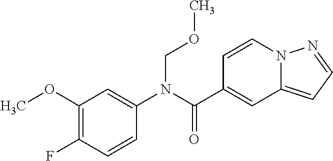 [SMILES: COCN(C(=O)c1ccn2nccc2c1)c1ccc(F)c(OC)c1]

To an ice-cooled solution of N-(4-fluoro-3-methoxy-phenyl)pyrazolo[1,5-a]pyridine-5-carboxamide (0.500 g, 1.58 mmol) in dry tetrahydrofuran (10.0 mL) was added sodium hydride 60% in oil (0.189 g, 7.89 mmol, 5.00 equiv.) under nitrogen. After 30 min of stirring, chloro(methoxy)methane (0.254 g, 3.15 mmol, 2.00 equiv.) was added and the reaction mixture was stirred at room temperature for 16 hours. The reaction mixture was cooled to 0° C., quenched with crushed ice and diluted with ethyl acetate. The combined organic layers were dried over sodium sulfate, filtered, and concentrated under reduced pressure. The crude residue was purified over a silica gel cartridge (cyclohexane/ethyl acetate) to afford N-(4-fluoro-3-methoxy-phenyl)-N-(methoxymethyl)pyrazolo[1,5-a]pyridine-5-carboxamide as an off white gummy solid.

LC-MS (method C): Rt 3.32 min, m/z=330 (M+H)⁺

¹H-NMR (400 MHz, DMSO-d⁶, ppm) δ=3.36 (s, 3H), 3.75 (s, 3H), 5.15 (2H), 6.68 (dd, 1H), 6.72-6.82 (m, 2H), 7.08-7.13 (m, 1H), 7.20-7.24 (dd, 1H), 7.79 (s, 1H), 8.01 (dd, 1H), 8.56-8.61 (dd, 1H)

### Step C: Preparation of N-(4-fluoro-3-methoxy-phenyl)-3-iodo-N-(methoxymethyl)pyrazolo[1,5-a]pyridine-5-carboxamide

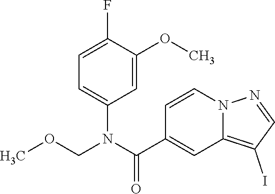 [SMILES: COCN(C(=O)c1ccn2ncc(I)c2c1)c1ccc(F)c(OC)c1]

To a solution of N-(4-fluoro-3-methoxy-phenyl)-N-(methoxymethyl)pyrazolo[1,5-a]pyridine-5-carboxamide (0.180 g, 0.492 mmol) in dimethylformamide (2.00 mL) was added N-Iodosuccinimide (0.122 g, 0.541 mmol, 1.10 equiv.). The reaction mixture was stirred at room temperature for 16 hours. The reaction mixture was poured into water and extracted with ethyl acetate. The combined organic layers were dried over sodium sulfate, filtered, and concentrated under reduced pressure. The crude residue was purified over a silica gel cartridge (cyclohexane/ethyl acetate) to afford N-(4-fluoro-3-methoxy-phenyl)-3-iodo-N-(methoxymethyl)pyrazolo[1,5-a]pyridine-5-carboxamide as an off-white gummy solid.

LC-MS (method C): Rt 4.00 min, m/z=456 (M+H)⁺

¹H-NMR (400 MHz, DMSO-d⁶, ppm) δ=3.38 (s, 3H), 3.78 (s, 3H), 5.18 (s, 2H), 6.80-6.90 (m, 2H), 7.05-7.15 (m, 1H), 7.30-7.33 (dd, 1H), 7.50-7.60 (s, 1H), 8.13 (s, 1H), 8.60-8.70 (dd, 1H)

### Step D: Preparation of methyl N-[5-[5-[(4-fluoro-3-methoxy-phenyl)-(methoxymethyl)carbamoyl]pyrazolo[1,5-a]pyridin-3-yl]-2-pyridyl]carbamate (Compound 1)

To a solution of N-(4-fluoro-3-methoxy-phenyl)-3-iodo-N-(methoxymethyl)pyrazolo[1,5-a]pyridine-5-carboxamide (0.400 g, 0.835 mmol.), methyl N-[5-(4,4,5,5-tetramethyl-1,3,2-dioxaborolan-2-yl)-2-pyridyl]carbamate (0.284 g, 1.00 mmol, 1.20 equiv.) in 1,4-dioxane:water (3:1, 5.00 mL) was added cesium carbonate (0.544 g, 1.67 mmol, 2.00 equiv.), the mixture was purged with a stream of argon for 2 minutes. cataCXium® A Pd G3 (CAS 1651823-59-4) (0.0304 g, 0.0417 mmol, 0.05 equiv.) was added to the reaction mixture under N₂ atmosphere. The resulting reaction mixture was irradiated in the microwaves at 100° C. for 1 hour. After cooling down to room temperature, the reaction mixture was filtered through a Celite pad and washed with ethyl acetate. The filtrate was washed with water, then brine, dried over sodium sulfate, filtered, and concentrated under reduced pressure. The crude residue was purified over silica gel cartridge (dichloromethane/methanol) to afford methyl N-[5-[5-[(4-fluoro-3-methoxy-phenyl)-(methoxymethyl)carbamoyl]pyrazolo[1,5-a]pyridin-3-yl]-2-pyridyl]carbamate as a yellow solid.

LC-MS (method C): Rt 3.31 min, m/z=480 (M+H)⁺

¹H-NMR (400 MHz, DMSO-d⁶, ppm) δ=3.38 (s, 3H), 3.70 (s, 3H), 3.78 (s, 3H), 5.19 (s, 2H), 6.80-6.87 (m, 1H), 6.88-6.93 (m, 1H), 7.07-7.13 (m, 1H), 7.31-7.40 (m, 1H), 7.83-7.90 (m, 1H), 7.90-7.97 (m, 2H), 8.42 (s, 1H), 8.51 (s, 1H), 8.60-8.70 (m, 1H), 10.25 (s, 1H)

### Example 2: Preparation of methyl N-[5-[5-[(4-chloro-3-methoxy-phenyl)-(cyanomethyl)carbamoyl]pyrazolo[1.5-a]pyridin-3-yl]-2-pyridyl]carbamate (Compound 2)

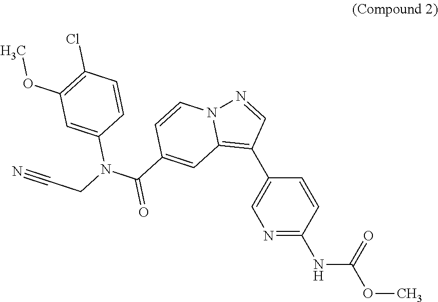 [SMILES: COC(=O)Nc1ccc(-c2cnn3ccc(C(=O)N(CC#N)c4ccc(Cl)c(OC)c4)cc23)cn1]

### Step A: Preparation of methyl pyrazolo[1,5-a]pyridine-5-carboxylate

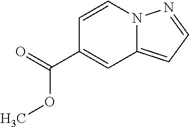

To a solution of pyrazolo[1,5-a]pyridine-5-carboxylic acid (CAS 104468-87-3, 3.50 g, 22.0 mmol) in methanol (88 mL) was added concentrated sulfuric acid (0.12 mL, 2.2 mmol, 0.10 equiv.). The reaction mixture was stirred at reflux for 16 hours. The reaction mixture was diluted in water and ethyl acetate and basified to pH 8 with sodium bicarbonate. The combined organic layers were dried over sodium sulfate, filtered, and concentrated under reduced pressure to afford methyl pyrazolo[1,5-a]pyridine-5-carboxylate beige solid.

LC-MS (method C): Rt 0.74 min, m/z=177 (M+H)⁺

¹H-NMR (400 MHz, CDCl₃, ppm) δ=3.93-4.02 (m, 3H), 6.68-6.80 (m, 1H), 7.30-7.37 (m, 1H), 7.99-8.08 (m, 1H), 8.31-8.37 (m, 1H), 8.46-8.54 (m, 1H)

### Step B: Preparation of methyl 3-bromopyrazolo[1,5-a]pyridine-5-carboxylate

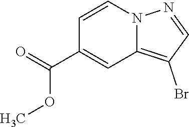

To a solution of methyl pyrazolo[1,5-a]pyridine-5-carboxylate (3.50 g, 14.0 mmol) in acetonitrile (85 mL) was added portionwise N-bromo succinimide (3.1 g, 18 mmol, 1.0 equiv.). The reaction mixture was stirred at room temperature for 5 hours. The reaction mixture was quenched with a sodium thiosulfate solution and then extracted with ethyl acetate. The combined organic layers were dried over sodium sulfate, filtered, and concentrated under reduced pressure. The crude residue was purified over silica gel cartridge (dichloromethane/methanol) to afford methyl 3-bromopyrazolo[1,5-a]pyridine-5-carboxylate white solid.

LC-MS (method C): Rt 0.93 min, m/z=255/257 (M+H)⁺

¹H-NMR (400 MHz, CDCl₃, ppm) δ=3.94-4.05 (m, 3H), 7.35-7.43 (m, 1H), 7.96-8.08 (m, 1H), 8.24-8.34 (m, 1H), 8.39-8.50 (m, 1H)

### Step C: Preparation of 3-bromopyrazolo[1,5-a]pyridine-5-carboxylic acid (Compound X-1)

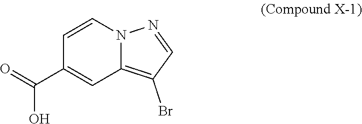 [SMILES: O=C(O)c1ccn2ncc(Br)c2c1]

To a solution of methyl 3-bromopyrazolo[1,5-a]pyridine-5-carboxylate (1.50 g, 5.88 mmol) in water (5.88 mL) and tetrahydrofuran (17.6 mL) was added lithium hydroxide (0.293 g, 11.8 mmol, 2.00 equiv.). The reaction mixture was stirred at room temperature for 16 hours. The reaction mixture was adjusted to pH 4 with 1 N hydrogen chloride, diluted with water and extracted with ethyl acetate. The combined organic layers were dried over sodium sulfate, filtered, and concentrated under reduced pressure to afford 3-bromopyrazolo[1,5-a]pyridine-5-carboxylic acid.

LC-MS (method C): Rt 0.74 min, m/z=241/243 (M+H)⁺

¹H-NMR (400 MHz, DMSO-d⁶, ppm) δ=7.17-7.46 (m, 1H), 7.98-8.20 (m, 1H), 8.26-8.37 (m, 1H), 8.72-8.88 (m, 1H), 13.38-13.70 (m, 1H)

### Step D: Preparation of 2-(4-chloro-3-methoxy-anilino)acetonitrile

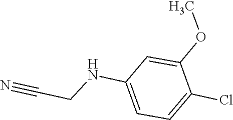

To a solution of 4-chloro-3-methoxy-aniline (10.5 g, 10 mmol) in acetonitrile (19 mL) were added 2-bromoacetonitrile (1.40 mL, 20.0 mmol, 2.00 equiv.), sodium iodide (0.710 g, 4.80 mmol, 0.50 equiv.) and potassium carbonate (1.600 g, 11.00 mmol, 1.20 equiv.). The reaction mixture was stirred at room temperature for 16 hours and then at reflux for 6 hours. The reaction mixture was filtered, diluted with 2 M sodium hydroxide and extracted with ethyl acetate. The combined organic layers were dried over sodium sulfate, filtered, and concentrated under reduced pressure. The crude residue was purified over silica gel cartridge (cyclohexane/ethyl acetate) to afford 2-(4-chloro-3-methoxy-anilino)acetonitrile as a brown oil.

LC-MS (method C): Rt 0.80 min, m/z=197/199 (M+H)⁺

¹H-NMR (400 MHz, CDCl₃, ppm) δ=3.87-3.92 (m, 3H), 3.96-4.05 (m, 1H), 4.09-4.14 (m, 2H), 6.23-6.31 (m, 2H), 7.22-7.26 (m, 1H)

### Step E: Preparation of 3-bromo-N-(4-chloro-3-methoxy-phenyl)-N-(cyanomethyl)pyrazolo[1,5-a]pyridine-5-carboxamide

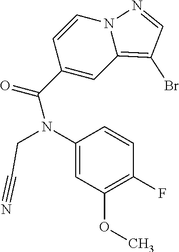 [SMILES: COc1cc(N(CC#N)C(=O)c2ccn3ncc(Br)c3c2)ccc1F]

To a solution of 2-(4-chloro-3-methoxy-anilino)acetonitrile (0.150 g, 0.763 mmol), 3-bromopyrazolo[1,5-a]pyridine-5-carboxylic acid (Compound X-1) (0.209 g, 0.839 mmol, 1.10 equiv.) and N-ethyl-N-isopropyl-propan-2-amine (0.522 mL, 3.05 mmol, 4.00 equiv.) in ethyl acetate (3.05 mL) was added 1-propanephosphonic anhydride 50% in ethyl acetate (0.500 mL, 0.839 mmol, 1.10 equiv.). The reaction mixture was stirred at 85° C. for 16 hours. The reaction mixture was diluted with ethyl acetate and quenched with water. The combined organic layers were dried over sodium sulfate, filtered, and concentrated under reduced pressure. The crude residue was purified over silica gel cartridge (cyclohexane/ethyl acetate) to afford 3-bromo-N-(4-chloro-3-methoxy-phenyl)-N-(cyanomethyl)pyrazolo[1,5-a]pyridine-5-carboxamide as a yellow gum

LC-MS (method C): Rt 0.98 min, m/z=419 (M+H)⁺

¹H-NMR (400 MHz, CDCl₃, ppm) δ=3.85 (s, 3H), 4.78 (s, 2H), 6.73 (dd, J=7.27, 1.82 Hz, 1H), 6.78-6.83 (m, 2H), 7.35-7.42 (m, 1H), 7.71 (dd, J=1.82, 0.73 Hz, 1H), 7.96 (s, 1H), 8.26 (dd, J=7.27, 0.73 Hz, 1H).

### Step F: Preparation of methyl N-[5-[5-[(4-chloro-3-methoxy-phenyl)-(cyanomethyl)carbamoyl]pyrazolo[1,5-a]pyridin-3-yl]-2-pyridyl]carbamate (Compound 2)

A mixture of 3-bromo-N-(4-chloro-3-methoxy-phenyl)-N-(cyanomethyl)pyrazolo[1,5-a]pyridine-5-carboxamide (0.074 g, 0.18 mmol), methyl N-[5-(4,4,5,5-tetramethyl-1,3,2-dioxaborolan-2-yl)-2-pyridyl]carbamate (0.069 g, 0.25 mmol, 1.4 equiv.) and cesium carbonate (0.086 g, 0.26 mmol, 1.50 eq.) in 2-methyltetrahydrofuran (1.1 mL) was flushed with argon for 5 min. Tetrakis(triphenylphosphine)palladium(0) (0.011 g, 0.0088 mmol, 0.050 eq.) was then added and the reaction mixture was heated under microwave irradiation at 100° C. for 30 minutes. The reaction mixture was cooled down to room temperature and then water was added. The aqueous layer was extracted with ethyl acetate and the combined organic layers were washed with brine, dried over sodium sulfate, filtered, and concentrated under reduced pressure. The crude residue was purified over a silica gel cartridge (cyclohexane/ethyl acetate) to afford methyl N-[5-[5-[(4-chloro-3-methoxy-phenyl)-(cyanomethyl)carbamoyl]pyrazolo[1,5-a]pyridin-3-yl]-2-pyridyl]carbamate as a beige solid.

LC-MS (method C): Rt 0.91 min, m/z=491 (M+H)⁺

¹H-NMR (400 MHz, CDCl₃, ppm) δ=3.86 (s, 3H), 3.88 (s, 3H), 4.78 (s, 2H), 6.78-6.88 (m, 3H), 7.42 (d, J=8.36 Hz, 1H), 7.61 (dd, J=8.72, 2.54 Hz, 1H), 7.67 (br d, J=1.45 Hz, 1H), 7.77-7.86 (m, 1H), 8.09 (d, J=8.72 Hz, 1H), 8.14 (s, 1H), 8.32 (d, J=1.00 Hz, 1H), 8.37 (d, J=7.27 Hz, 1H)

### Example 3: Preparation of methyl N-[5-[5-[cyanomethyl-(4-fluoro-3-methoxy-phenyl)carbamoyl]pyrazolo[1,5-a]pyridin-3-yl]-2-pyridyl]carbamate (Compound 31

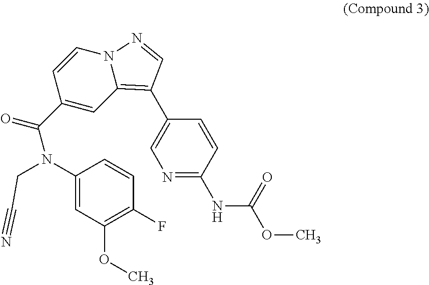 [SMILES: COC(=O)Nc1ccc(-c2cnn3ccc(C(=O)N(CC#N)c4ccc(F)c(OC)c4)cc23)cn1]

### Step A: Preparation of 2-(4-fluoro-3-methoxy-anilino)acetonitrile

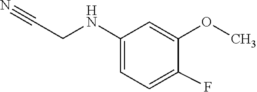

To a solution of 4-fluoro-3-methoxy-aniline (1.5 g, 11 mmol) in acetonitrile (21 mL) were added 2-bromoacetonitrile (0.78 mL, 11 mmol, 1.1 equiv.), sodium iodide (0.800 g, 5.30 mmol 0.50 equiv.) and potassium carbonate (1.80 g, 13.0 mmol, 1.20 equiv.). The reaction mixture was stirred at reflux for 16 hours. The reaction mixture was filtered, diluted with ethyl acetate and quenched with a saturated solution of sodium bicarbonate. The combined organic layers were dried over sodium sulfate, filtered, and concentrated under reduced pressure. The crude residue was purified over silica gel cartridge (cyclohexane/ethyl acetate) to afford 2-(4-fluoro-3-methoxy-anilino)acetonitrile as an off-white solid.

LC-MS (method C): Rt 0.71 min, m/z=181 (M+H)⁺

¹H-NMR (400 MHz, CDCl₃, ppm) δ=3.79-3.85 (m, 1H), 3.86 (s, 3H), 4.07 (d, J=6.90 Hz, 2H), 6.40-6.49 (m, 1H), 6.51-6.60 (m, 1H), 6.87-6.98 (m, 1H).

### Step B: Preparation of 3-bromo-N-(cyanomethyl)-N-(4-fluoro-3-methoxy-phenyl)pyrazolo[1,5-a]pyridine-5-carboxamide

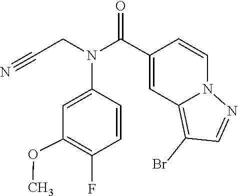 [SMILES: COc1cc(N(CC#N)C(=O)c2ccn3ncc(Br)c3c2)ccc1F]

To a solution of 2-(4-fluoro-3-methoxy-anilino)acetonitrile (0,150 g, 0,833 mmol), 3-bromopyrazolo[1,5-a]pyridine-5-carboxylic acid (Compound X-1) (0.228 g, 0.916 mmol, 1.10 equiv.) and N-ethyl-N-isopropyl-propan-2-amine (0.570 mL, 3.33 mmol, 4.00 equiv.) in ethyl acetate (3.33 mL) was added 1-propanephosphonic anhydride 50% in ethyl acetate (0.546 mL, 0.916 mmol, 1.10 equiv.). The reaction mixture was stirred at 85° C. for 16 hours. The reaction mixture was diluted with ethyl acetate and quenched with water. The combined organic layers were dried over sodium sulfate, filtered, and concentrated under reduced pressure. The crude residue was purified over silica gel cartridge (cyclohexane/ethyl acetate) to afford 3-bromo-N-(cyanomethyl)-N-(4-fluoro-3-methoxy-phenyl)pyrazolo[1,5-a]pyridine-5-carboxamide as a yellow gum.

LC-MS (method C): Rt 0.94 min, m/z=403 (M+H)⁺

1H-NMR (400 MHz, CDCl₃, ppm) δ=3.85 (s, 3H), 4.77 (s, 2H), 6.73-6.90 (m, 3H), 7.09 (dd, J=10.54, 8.36 Hz, 1H), 7.67 (dd, J=1.82, 1.09 Hz, 1H), 7.96 (s, 1H), 8.26 (dd, J=7.27, 1.09 Hz, 1H)

### Step F: Preparation of methyl N-[5-[5-[cyanomethyl-(4-fluoro-3-methoxy-phenyl)carbamoyl]pyrazolo[1,5-a]pyridin-3-yl]-2-pyridyl]carbamate (Compound 3)

A mixture of 3-bromo-N-(cyanomethyl)-N-(4-fluoro-3-methoxy-phenyl)pyrazolo[1,5-a]pyridine-5-carboxamide (0.23 g, 0.57 mmol), methyl N-[5-(4,4,5,5-tetramethyl-1,3,2-dioxaborolan-2-yl)-2-pyridyl]carbamate (0.22 g, 0.80 mmol, 1.4 equiv.) and cesium carbonate (0.28 g, 0.86 mmol, 1.5 equiv.) in 2-methyltetrahydrofuran (3.4 mL) was flushed with argon for 5 min. Tetrakis(triphenylphosphine)palladium(0) (0.034 g, 0.029 mmol, 0.05 equiv.) was then added and the reaction mixture was heated under microwave irradiation at 100° C. for 30 minutes. The reaction mixture was cooled down to room temperature and then water was added. The aqueous layer was extracted with ethyl acetate and the combined organic layers were washed with brine, dried over sodium sulfate, filtered, and concentrated under reduced pressure. The crude residue was purified over a silica gel cartridge (cyclohexane/ethyl acetate) to afford methyl N-[5-[5-[cyanomethyl-(4-fluoro-3-methoxy-phenyl)carbamoyl]pyrazolo[1,5-a]pyridin-3-yl]-2-pyridyl]carbamate as a yellow solid.

LC-MS (method C): Rt 0.87 min, m/z=474 (M+H)⁺

¹H-NMR (400 MHz, CDCl₃, ppm) δ=3.73-3.91 (m, 6H), 4.68-4.82 (m, 2H), 6.74-6.81 (m, 1H), 6.81-6.86 (m, 1H), 6.86-6.93 (m, 1H), 7.04-7.16 (m, 1H), 7.42-7.74 (m, 2H), 7.78-7.83 (m, 1H), 8.04-8.11 (m, 1H), 8.11-8.16 (m, 1H), 8.28-8.33 (m, 1H), 8.33-8.38 (m, 1H)

### Example 4: Preparation of methyl N-[5-[5-[(4-fluoro-3-methoxy-phenyl)-methyl-carbamoyl]pyrazolo[1,5-a]pyridin-3-yl]-2-pyridyl]carbamate (Compound 4)

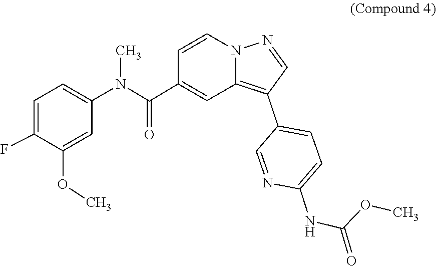 [SMILES: COC(=O)Nc1ccc(-c2cnn3ccc(C(=O)N(C)c4ccc(F)c(OC)c4)cc23)cn1]

### Step A: Preparation of 3-bromo-N-(4-fluoro-3-methoxy-phenyl)-N-methyl-pyrazolo[1,5-a]pyridine-5-carboxamide

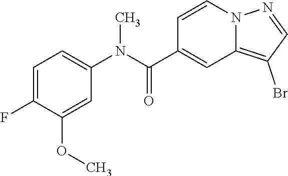 [SMILES: COc1cc(N(C)C(=O)c2ccn3ncc(Br)c3c2)ccc1F]

To a solution of 3-bromopyrazolo[1,5-a]pyridine-5-carboxylic acid (Compound X-1) (0.702 g, 2.77 mmol), 4-fluoro-3-methoxy-N-methylaniline (0.599 mL, 4.15 mmol, 1.50 equiv.) and N-ethyl-N-isopropyl-propan-2-amine (2.37 mL, 13.8 mmol, 5.00 equiv.) in N,N-dimethylacetamide (17.6 mL) was added 1-propanephosphonic anhydride 50% in ethyl acetate (4.12 mL, 6.92 mmol, 2.50 equiv.). The reaction mixture was stirred at 55° C. for 35 minutes. The reaction mixture was diluted with ethyl acetate and quenched with a saturated solution of sodium carbonate. The combined organic layers were dried over sodium sulfate, filtered, and concentrated under reduced pressure. The crude residue was purified over silica gel cartridge (cyclohexane/ethyl acetate:ethanol, 3:1) to afford 3-bromo-N-(4-fluoro-3-methoxy-phenyl)-N-methyl-pyrazolo[1,5-a]pyridine-5-carboxamide.

LC-MS (method C): Rt 0.83 min, m/z=378/380 (M+H)⁺

¹H-NMR (400 MHz, DMSO-d⁶, ppm) δ=3.36-3.48 (m, 3H), 3.70-3.81 (m, 3H), 6.73-6.86 (m, 1H), 7.02-7.14 (m, 1H), 7.15-7.23 (m, 1H), 7.29-7.36 (m, 1H), 7.46-7.55 (m, 1H), 7.67-7.79 (m, 1H), 8.17-8.35 (m, 1H)

### Step B: Preparation of methyl N-[5-[5-[(4-fluoro-3-methoxy-phenyl)-methyl-carbamoyl]pyrazolo[1,5-a]pyridin-3-yl]-2-pyridyl]carbamate (Compound 4)

A mixture of 3-bromo-N-(4-fluoro-3-methoxy-phenyl)-N-methyl-pyrazolo[1,5-a]pyridine-5-carboxamide (0.20 g, 0.53 mmol), 2-methoxycarbonylaminopyridine-5-boronic acid pinacol ester (0.216 g, 0,740 mmol, 1.40 equiv.) and cesium carbonate (0.26 g, 0.79 mmol, 1.5 equiv.) in 2-methyltetrahydrofuran (3.2 mL) was flushed with argon for 5 min. Tetrakis(triphenylphosphine)palladium(0) (0.032 g, 0.026 mmol, 0.050 eq.) was then added and the reaction mixture was heated under microwave irradiation at 100° C. for 1 hour. The reaction mixture was cooled down to room temperature and then water was added. The aqueous layer was extracted with ethyl acetate and the combined organic layers were washed with brine, dried over sodium sulfate, filtered, and concentrated under reduced pressure. The crude residue was purified over a silica gel cartridge (cyclohexane/ethyl acetate) to afford methyl N-[5-[5-[(4-fluoro-3-methoxy-phenyl)-methyl-carbamoyl]pyrazolo[1,5-a]pyridin-3-yl]-2-pyridyl]carbamate.

LC-MS (method C): Rt 0.87 min, m/z=450 (M+H)⁺

¹H-NMR (400 MHz, CDCl₃, ppm) δ=3.45-3.55 (m, 3H), 3.75-3.83 (m, 3H), 3.83-3.91 (m, 3H), 6.60-6.72 (m, 1H), 6.72-6.83 (m, 2H), 6.96-7.06 (m, 1H), 7.62-7.71 (m, 1H), 7.71-7.79 (m, 1H), 8.03-8.10 (m, 1H), 8.10-8.14 (m, 1H), 8.19-8.28 (m, 1H), 8.28-8.35 (m, 1H), 8.35-8.40 (m, 1H)

### Example 5: Preparation of 3-(4-acetamidophenyl)-N-(4-chlorophenyl)-N-methyl-pyrazolo[1,5-a]pyridine-5-carboxamide (Compound 6)

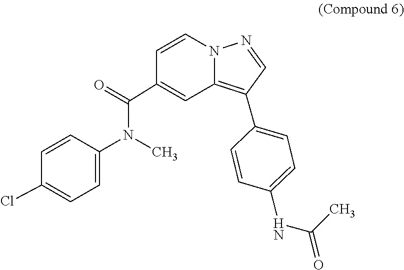 [SMILES: CC(=O)Nc1ccc(-c2cnn3ccc(C(=O)N(C)c4ccc(Cl)cc4)cc23)cc1]

### Step A: Preparation of N-(4-chlorophenyl)pyrazolo[1,5-a]pyridine-5-carboxamide

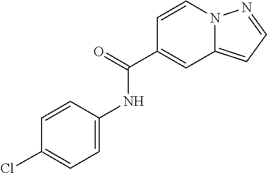 [SMILES: O=C(Nc1ccc(Cl)cc1)c1ccn2nccc2c1]

To a solution of pyrazolo[1,5-a]pyridine-5-carboxylic acid (CAS 104468-87-3) (2.00 g, 12.3 mmol), 4-chloroaniline (1.97 g, 14.8 mmol, 1.20 equiv.) and diisopropyl amine (6.46 mL, 37.0 mmol, 3.00 equiv.) in dimethylformamide (37.0 mL) was added portionwise 1-[bis(dimethylamino)methylene]-1H-1,2,3-triazolo[4,5-b]pyridinium 3-oxide hexafluorophosphate (4.79 g, 12.3 mmol, 1.00 equiv.). The reaction mixture was stirred at room temperature for 2 hours. The reaction mixture was quenched with an aqueous solution of sodium carbonate and extracted with ethyl acetate. The combined organic layers were washed with brine, dried over sodium sulfate, filtered, and concentrated under reduced pressure. The crude residue was purified over a silica gel cartridge (cyclohexane/ethyl acetate) to afford N-(4-chlorophenyl)pyrazolo[1,5-a]pyridine-5-carboxamide as a white solid.

LC-MS (method C): Rt 0.91 min, m/z=272/274 (M+H)⁺

¹H-NMR (400 MHz, CDCl₃, ppm) δ=6.70-6.78 (m, 1H), 7.16-7.23 (m, 1H), 7.33-7.40 (m, 2H), 7.58-7.65 (m, 2H), 7.77-7.87 (m, 1H), 8.04-8.10 (m, 1H), 8.10-8.14 (m, 1H), 8.52-8.60 (m, 1H)

### Step B: Preparation of N-(4-chlorophenyl)-N-methyl-pyrazolo[1,5-a]pyridine-5-carboxamide

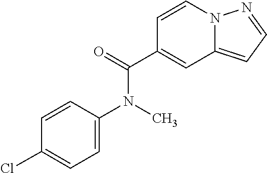 [SMILES: CN(C(=O)c1ccn2nccc2c1)c1ccc(Cl)cc1]

To an ice-cooled solution of N-(4-chlorophenyl)pyrazolo[1,5-a]pyridine-5-carboxamide (1.50 g, 5.52 mmol) in tetrahydrofuran (13.8 mL) was added portionwise sodium hydride (0,265 g, 6.07 mmol, 1.10 equiv.). After 10 min at 0° C. was added dropwise iodomethane (0,526 mL, 8.28 mmol, 1.50 equiv.) The reaction mixture was stirred at room temperature for 4 hours. Water was carefully added and the reaction mixture was diluted with ethyl acetate. The combined organic layers were washed with brine, dried over sodium sulfate, filtered, and concentrated under reduced pressure. The crude residue was purified over a silica gel cartridge (cyclohexane/ethyl acetate) to afford N-(4-chlorophenyl)-N-methyl-pyrazolo[1,5-a]pyridine-5-carboxamide as a yellow oil.

LC-MS (method C): Rt 0.88 min, m/z=286/288 (M+H)⁺

¹H-NMR (400 MHz, CDCl₃, ppm) δ=3.46-3.54 (m, 3H), 6.50-6.55 (m, 1H), 6.55-6.63 (m, 1H), 7.01-7.12 (m, 2H), 7.20-7.30 (m, 2H), 7.51-7.65 (m, 1H), 7.89-7.99 (m, 1H), 8.20-8.29 (m, 1H).

### Step C: Preparation of 3-bromo-N-(4-chlorophenyl)-N-methyl-pyrazolo[1,5-a]pyridine-5-carboxamide

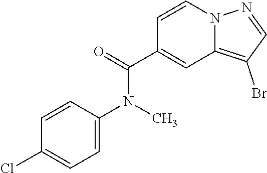 [SMILES: CN(C(=O)c1ccn2ncc(Br)c2c1)c1ccc(Cl)cc1]

To a solution of N-(4-chlorophenyl)-N-methyl-pyrazolo[1,5-a]pyridine-5-carboxamide (1.10 g, 3.85 mmol) in acetonitrile (19.3 mL) was added portionwise N-bromosuccinimide (0.706 g, 3.97 mmol, 1.03 equiv.). The reaction mixture was stirred at room temperature for 3 hours. The reaction mixture was quenched with a sodium sulfate solution and extracted with ethyl acetate. The combined organic layers were washed with brine, dried over sodium sulfate, filtered, and concentrated under reduced pressure. The crude residue was purified over a silica gel cartridge (cyclohexane/ethyl acetate) to afford 3-bromo-N-(4-chlorophenyl)-N-methyl-pyrazolo[1,5-a]pyridine-5-carboxamide as a solid.

LC-MS (method C): Rt 1.00 min, m/z=364/366 (M+H)⁺

¹H-NMR (400 MHz, CDCl₃, ppm) δ=3.42-3.54 (m, 3H), 6.53-6.67 (m, 1H), 7.03-7.12 (m, 2H), 7.27-7.32 (m, 2H), 7.53-7.64 (m, 1H), 7.84-7.96 (m, 1H), 8.13-8.25 (m, 1H).

### Step D: Preparation of 3-(4-aminophenyl)-N-(4-chlorophenyl)-N-methyl-pyrazolo[1,5-a]pyridine-5-carboxamide

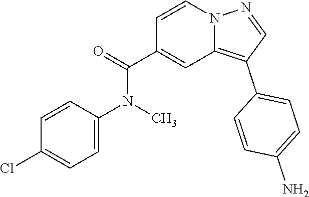 [SMILES: CN(C(=O)c1ccn2ncc(-c3ccc(N)cc3)c2c1)c1ccc(Cl)cc1]

To a mixture of 3-bromo-N-(4-chlorophenyl)-N-methyl-pyrazolo[1,5-a]pyridine-5-carboxamide (1.20 g, 3.29 mmol), 4-aminophenylboronic acid hydrochloride (0.685 g, 3.95 mmol, 1.20 equiv.) and sodium carbonate (1.05 g, 9.87 mmol, 3.00 equiv.) in 1,2-dimethoxyethane (26.3 mL) and water (6.58 mL) was added tetrakis(triphenylphosphine)palladium(0) (0.305 g, 0.263 mmol). The reaction mixture was stirred at 85° C. for 19 hours. The reaction mixture was diluted with water and extracted with ethyl acetate. The combined organic layers were washed with brine, dried over sodium sulfate, filtered, and concentrated under reduced pressure. The crude residue was purified over a silica gel cartridge (cyclohexane/ethyl acetate) to afford 3-(4-aminophenyl)-N-(4-chlorophenyl)-N-methyl-pyrazolo[1,5-a]pyridine-5-carboxamide as a yellow solid.

LC-MS (method C): Rt 0.84 min. m/z=377/379 (M+H)⁺

¹H-NMR (400 MHz, CDCl₃, ppm) δ=3.47-3.55 (m, 3H), 3.70-3.84 (m, 2H), 6.67-6.81 (m, 3H), 7.05-7.13 (m, 4H), 7.30-7.37 (m, 2H), 7.63-7.69 (m, 1H), 7.97-8.05 (m, 1H), 8.22-8.29 (m, 1H)

### Step E: Preparation of 3-(4-acetamidophenyl)-N-(4-chlorophenyl)-N-methyl-pyrazolo[1,5-a]pyridine-5-carboxamide (Compound 6)

To a solution of 3-(4-aminophenyl)-N-(4-chlorophenyl)-N-methyl-pyrazolo[1,5-a]pyridine-5-carboxamide (0.12 g, 0.32 mmol, 1.0 equiv.) and triethylamine (0.0673 mL, 0.478 mmol, 1.50 equiv.) in dichloromethane (2.5 mL) was added dropwise acetyl chloride (0.026 mL, 0.35 mmol, 1.1 equiv.). This reaction mixture was stirred at room temperature for 18 hours. The reaction mixture was diluted with dichloromethane and washed a saturated solution of sodium carbonate. The combined organic layers were washed with brine, dried over sodium sulfate, filtered, and concentrated under reduced pressure. The crude residue was purified over a silica gel cartridge (cyclohexane/ethyl acetate) to afford 3-(4-acetamidophenyl)-N-(4-chlorophenyl)-N-methyl-pyrazolo[1,5-a]pyridine-5-carboxamide.

LC-MS (method C): Rt 0.89 min, m/z=419 (M+H)⁺

¹H-NMR (400 MHz, CDCl₃, ppm) δ=2.20-2.31 (m, 3H), 3.47-3.55 (m, 3H), 6.72-6.80 (m, 1H), 7.05-7.13 (m, 2H), 7.16-7.23 (m, 1H), 7.26-7.27 (m, 2H), 7.31-7.37 (m, 2H), 7.52-7.61 (m, 2H), 7.65-7.72 (m, 1H), 8.03-8.11 (m, 1H), 8.25-8.35 (m, 1H)

### Example 6: Preparation of methyl N-[5-[5-[(2-methoxy-4-pyridyl)-methyl-carbamoyl]pyrazolo[1,5-a]pyridin-3-yl]-2-pyridyl]carbamate (Compound 17)

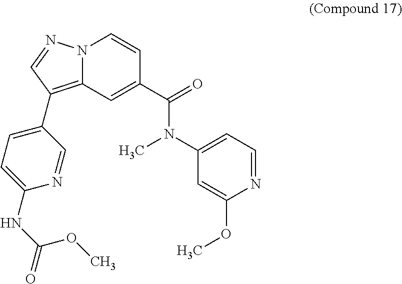 [SMILES: COC(=O)Nc1ccc(-c2cnn3ccc(C(=O)N(C)c4ccnc(OC)c4)cc23)cn1]

### Step A: Preparation of 3-bromo-N-(2-methoxy-4-pyridyl)-N-methyl-pyrazolo[1,5-a]pyridine-5-carboxamide

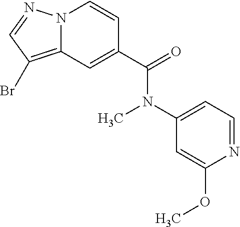 [SMILES: COc1cc(N(C)C(=O)c2ccn3ncc(Br)c3c2)ccn1]

POCl₃ (0.088 mL, 0.96 mmol, 3.0 equiv) was added to a solution of 3-bromopyrazolo[1,5-a]pyridine-5-carboxylic acid (Compound X-1) (77.1 mg, 0.320 mmol) and 2-methoxy-N-methyl-pyridin-4-amine (52.5 mg, 0.380 mmol, 1.19 equiv) in pyridine (1.00 mL) cooled down to 10° C. the reaction mixture was stirred at 10° C. for 2 hours. The reaction was quenched with water, then the mixture was concentrated under reduced pressure. The residue was dissolved in dimethylacetamide and filtered over silica gel. The crude residue was used in the next step without purification.

### Step B: Preparation of methyl N-[5-[5-[(2-methoxy-4-pyridyl)-methyl-carbamoyl]pyrazolo[1,5-a]pyridin-3-yl]-2-pyridyl]carbamate (Compound 17)

A solution of Na₂CO₃ (12.85 mg, 0.1200 mmol, 3.00 equiv.) in water (0.20 mL) was degassed and added to a solution of methyl N-[5-(4,4,5,5-tetramethyl-1,3,2-dioxaborolan-2-yl)-2-pyridyl]carbamate (14.5 mg, 0.0521 mmol, 1.30 equiv), 3-bromo-N-(2-methoxy-4-pyridyl)-N-methyl-pyrazolo[1,5-a]pyridine-5-carboxamide (14.4 mg, 0.0401 mmol) and XPhos Pd G2 (3.31 mg, 0.00421 mmol, 0.010 equiv.) in degassed acetonitrile (0.30 mL) inside of a glovebox. The reaction mixture was then stirred at 100° C. for 16 hours. The solvent was then removed. The residue was taken up in dimethylacetamide/methanol (1:1, 0.800 mL) and filtered over silica gel, then purified by reverse phase chromatography (eluting water/acetonitrile with 0.1% formic acid) to afford methyl N-[5-[5-[(2-methoxy-4-pyridyl)-methyl-carbamoyl]pyrazolo[1,5-a]pyridin-3-yl]-2-pyridyl]carbamate.

LC-MS (method B): Rt 1.16 min, m/z=433 (M+H)⁺

### Example 7: Preparation of 3-[6-(ethylcarbamoylamino)-3-pyridyl]-N-(3-fluorophenyl)-N-methyl-pyrazolo[1,5-a]pyridine-5-carboxamide (Compound 26)

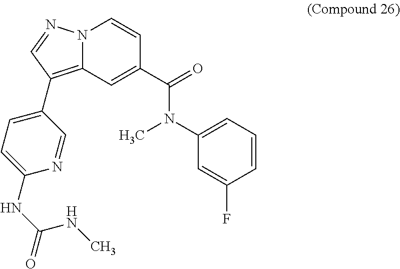 [SMILES: CNC(=O)Nc1ccc(-c2cnn3ccc(C(=O)N(C)c4cccc(F)c4)cc23)cn1]

### Step A: Preparation of 3-bromo-N-(3-fluorophenyl)-N-methyl-pyrazolo[1,5-a]pyridine-5-carboxamide

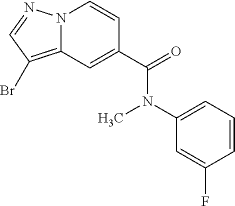 [SMILES: CN(C(=O)c1ccn2ncc(Br)c2c1)c1cccc(F)c1]

POCl₃ (0.088 mL, 0.96 mmol, 3.0 equiv) was added to a solution of 3-bromopyrazolo[1,5-a]pyridine-5-carboxylic acid (Compound X-1) (77.1 mg, 0.320 mmol) and 3-fluoro-N-methylaniline (48.6 mg, 0.389 mmol, 1.22 equiv) in pyridine (1.00 mL) cooled down to 10° C. the reaction mixture was stirred at 10° C. for 2 hours. The reaction was quenched with water, then the mixture was concentrated under reduced pressure. The residue was dissolved in dimethylacetamide and filtered over silica gel. The crude residue was used in the next step without purification.

### Step B: Preparation of 3-[6-(ethylcarbamoylamino)-3-pyridyl]-N-(3-fluorophenyl)-N-methyl-pyrazolo[1,5-a]pyridine-5-carboxamide (Compound 26)

A solution of Na₂CO₃ (12.85 mg, 0.1200 mmol, 3.00 equiv.) in water (0.20 mL) was degassed and added to a solution of 3-ethyl-1-[5-(4,4,5,5-tetramethyl-1,3,2-dioxaborolan-2-yl)pyridin-2-yl]urea (15.0 mg, 0.051 mmol, 1.28 equiv), 3-bromo-N-(3-fluorophenyl)-N-methyl-pyrazolo[1,5-a]pyridine-5-carboxamide (13.9 mg, 0.0401 mmol) and XPhos Pd G2 (3.31 mg, 0.00421 mmol, 0.010 equiv.) in degassed acetonitrile (0.30 mL) inside of a glovebox. The reaction mixture was then stirred at 100° C. for 16 hours. The solvent was then removed. The residue was taken up in dimethylacetamide/methanol (1:1, 0.800 mL) and filtered over silica gel, then purified by reverse phase chromatography (eluting water/acetonitrile with 0.1% formic acid) to afford 3-[6-(ethylcarbamoylamino)-3-pyridyl]-N-(3-fluorophenyl)-N-methyl-pyrazolo[1,5-a]pyridine-5-carboxamide.

LC-MS (method B): Rt 1.25 min, m/z=433 (M+H)⁺

### Example 8: Preparation of 3-(6-acetamido-3-pyridyl)-N-(4-cyano-3-methoxy-phenyl)-N-methyl-pyrazolo[1,5-a]pyridine-5-carboxamide (Compound 41)

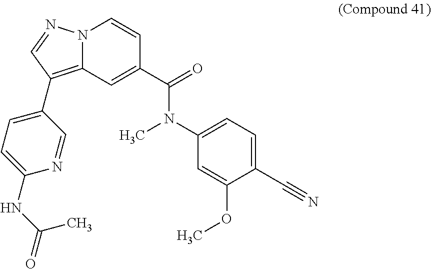 [SMILES: COc1cc(N(C)C(=O)c2ccn3ncc(-c4ccc(NC(C)=O)nc4)c3c2)ccc1C#N]

### Step A: Preparation of 3-bromo-N-(4-cyano-3-methoxy-phenyl)-N-methyl-pyrazolo[1,5-a]pyridine-5-carboxamide

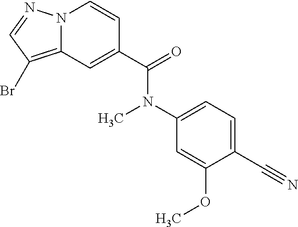 [SMILES: COc1cc(N(C)C(=O)c2ccn3ncc(Br)c3c2)ccc1C#N]

POCl₃ (0.088 mL, 0.96 mmol, 3.0 equiv) was added to a solution of 3-bromopyrazolo[1,5-a]pyridine-5-carboxylic acid (Compound X-1) (77.1 mg, 0.320 mmol) and 2-methoxy-4-(methylamino)benzonitrile (58.1 mg, 0.358 mmol, 1.12 equiv) in pyridine (1.00 mL) cooled down to 10° C. The reaction mixture was stirred at 10° C. for 2 hours. The reaction was quenched with water, then the mixture was concentrated under reduced pressure. The residue was dissolved in dimethylacetamide and filtered over silica gel. The crude residue was used in the next step without purification.

### Step B: Preparation of 3-(6-acetamido-3-pyridyl)-N-(4-cyano-3-methoxy-phenyl)-N-methyl-pyrazolo[1,5-a]pyridine-5-carboxamide (Compound 41)

A solution of Na₂CO₃ (12.85 mg, 0.1200 mmol, 3.00 equiv.) in water (0.20 mL) was degassed and added to a solution of 2-acetamidopyridine-5-boronic acid pinacol ester (13.6 mg, 0.052 mmol, 1.30 equiv), 3-bromo-N-(4-cyano-3-methoxy-phenyl)-N-methyl-pyrazolo[1,5-a]pyridine-5-carboxamide (15.4 mg, 0.0401 mmol) and XPhos Pd G2 (3.31 mg, 0.00421 mmol, 0.010 equiv.) in degassed acetonitrile (0.30 mL) inside of a glovebox. The reaction mixture was then stirred at 100° C. for 16 hours. The solvent was then removed. The residue was taken up in dimethylacetamide/methanol (1:1, 0.800 mL) and filtered over silica gel, then purified by reverse phase chromatography (eluting water/acetonitrile with 0.1% formic acid) to afford 3-(6-acetamido-3-pyridyl)-N-(4-cyano-3-methoxy-phenyl)-N-methyl-pyrazolo[1,5-a]pyridine-5-carboxamide.

LC-MS (method B): Rt 1.16 min, m/z=441 (M+H)⁺

### Example 9: Preparation of N-(4-cyano-3-methyl-phenyl)-3-[4-[(2-methoxyacetyl)amino]phenyl]-N-methyl-pyrazolo[1,5-a]pyridine-5-carboxamide (Compound 45)

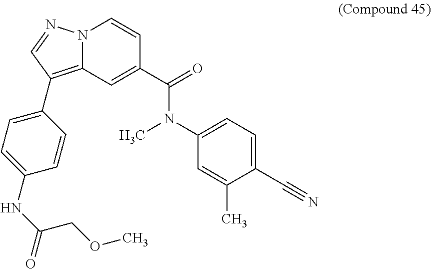 [SMILES: COCC(=O)Nc1ccc(-c2cnn3ccc(C(=O)N(C)c4ccc(C#N)c(C)c4)cc23)cc1]

### Step A: Preparation of 3-bromo-N-(4-cyano-3-methyl-phenyl)-N-methyl-pyrazolo[1,5-a]pyridine-5-carboxamide

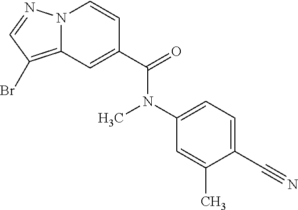 [SMILES: Cc1cc(N(C)C(=O)c2ccn3ncc(Br)c3c2)ccc1C#N]

POCl₃ (0.088 mL, 0.96 mmol, 3.0 equiv) was added to a solution of 3-bromopyrazolo[1,5-a]pyridine-5-carboxylic acid (Compound X-1) (77.1 mg, 0.320 mmol) and 2-methyl-4-(methylamino)benzonitrile (52.3 mg, 0.358 mmol, 1.12 equiv) in pyridine (1.00 mL) cooled down to 10° C. The reaction mixture was stirred at 10° C. for 2 hours. The reaction was quenched with water, then the mixture was concentrated under reduced pressure. The residue was dissolved in dimethylacetamide and filtered over silica gel. The crude residue was used in the next step without purification.

### Step B: Preparation of N-(4-cyano-3-methyl-phenyl)-3-[4-[(2-methoxyacetyl)amino]phenyl]-N-methyl-pyrazolo[1.5-a]pyridine-5-carboxamide (Compound 45)

A solution of Na₂CO₃ (12.85 mg, 0.1200 mmol, 3.00 equiv.) in water (0.20 mL) was degassed and added to a solution of 2-methoxy-N-[4-(4,4,5,5-tetramethyl-1,3,2-dioxaborolan-2-yl)phenyl]acetamide (15.6 mg, 0.054 mmol, 1.33 equiv), 3-bromo-N-(2-methoxy-4-pyridyl)-N-methyl-pyrazolo[1,5-a]pyridine-5-carboxamide (14.8 mg, 0.0401 mmol) and XPhos Pd G2 (3.31 mg, 0.00421 mmol, 0.010 equiv.) in degassed acetonitrile (0.30 mL) inside of a glovebox. The reaction mixture was then stirred at 100° C. for 16 hours. The solvent was then removed. The residue was taken up in dimethylacetamide/methanol (1:1, 0.800 mL) and filtered over silica gel, then purified by reverse phase chromatography (eluting water/acetonitrile with 0.1% formic acid) to afford N-(4-cyano-3-methyl-phenyl)-3-[4-[(2-methoxyacetyl)amino]phenyl]-N-methyl-pyrazolo[1,5-a]pyridine-5-carboxamide.

LC-MS (method B): Rt 1.29 min, m/z=454 (M+H)⁺

### Biological Examples

The fungicidal activity of the compounds of the invention have been tested as follows:

Phytophthora infestans/Tomato/Leaf Disc Preventative (Late Blight)

Tomato leaf disks are placed on water agar in multiwell plates (24-well format) and sprayed with the formulated test compound diluted in water. The leaf disks are inoculated with a spore suspension of the fungus 1 day after application. The inoculated leaf disks are incubated at 16° C. and 75% rh under a light regime of 24 h darkness followed by 12 h light/12 h darkness in a climate cabinet and the activity of a compound is assessed as percent disease control compared to untreated when an appropriate level of disease damage appears in untreated check leaf disks (5-7 days after application).

The following compounds gave at least 80% control of Phytophthora infestans at 200 ppm when compared to untreated control under the same conditions, which showed extensive disease development:
1, 2, 3, 4
   Plasmopara viticola/Grape/Leaf Disc Preventative (Late Blight)

Grape vine leaf disks are placed on water agar in multiwell plates (24-well format) and sprayed with the formulated test compound diluted in water. The leaf disks are inoculated with a spore suspension of the fungus 1 day after application. The inoculated leaf disks are incubated at 19° C. and 80% rh under a light regime of 12 h light/12 h darkness in a climate cabinet and the activity of a compound is assessed as percent disease control compared to untreated when an appropriate level of disease damage appears in untreated check leaf disks (6-8 days after application).

The following compounds gave at least 80% control of Plasmopara viticola at 200 ppm when compared to untreated control under the same conditions, which showed extensive disease development:
1, 3, 4
   Pythium ultimum/Liquid Culture (Seedling Damping Off)

Mycelia fragments and oospores of a newly grown liquid culture of the fungus are directly mixed into nutrient broth (PDB potato dextrose broth). After placing a (DMSO) solution of test compound into a microtiter plate (96-well format), the nutrient broth containing the fungal mycelia/spore mixture is added. The test plates are incubated at 24° C. and the inhibition of growth is determined photometrically 2-3 days after application.

The following compounds gave at least 80% control of Pythium ultimum at 20 ppm when compared to untreated control under the same conditions, which showed extensive disease development:
1, 2, 4, 5, 6

1. A compound of formula (I) 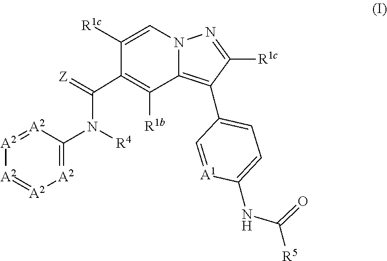
wherein Z is O or S, and preferably Z is O;
A¹ is CH or N, and preferably N;
R^{1a}, R^{1b} and R^{1c} are independently selected from hydrogen, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl;
A² are independently CR² or N, with the proviso that no more than three A² are N, preferably no more than two A² are N, preferably no more than one A² is N, and more preferably the four A² are CR²;
R² are independently selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen, hydroxy, and CN;
A³ is independently CR³ or N;
R³ is selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, amino, C₁₋₆alkylamino, diC₁₋₆-alkylamino, and C₃₋₆cycloalkylamino, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, amino, C₁₋₆alkylamino, diC₁₋₆-alkylamino, and C₃₋₆-cycloalkylamino groups is optionally substituted with one to three substituents independently selected from halogen, hydroxy, and CN;
R⁴ is selected from C₁₋₆alkyl, C₁₋₆alkoxy-C₁₋₆alkyl, C₃₋₆cycloalkyl, C₃₋₆cycloalkyl-C₁₋₄alkyl, C₂₋₆ alkenyl, C₂₋₆alkynyl, C₁₋₆alkoxy, C₁₋₆alkylsulfanyl-C₁₋₆alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl, diC₁₋₆alkylaminocarbonyl-C₁₋₆alkyl, and CN, wherein each of the C₁₋₆alkyl, C₁₋₆alkoxy-C₁₋₆alkyl, C₃₋₆-cycloalkyl, C₃₋₆cycloalkyl-C₁₋₄alkyl, C₂₋₆alkenyl, C₂₋₆alkynyl, C₁₋₆alkoxy, C₁₋₆alkylsulfanyl-C₁₋₆ alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl and diC₁₋₆alkylaminocarbonyl-C₁₋₆alkyl groups is optionally substituted with one to three substituents independently selected from halogen and CN;
wherein A³ and R⁴ taken together optionally form a ring, preferably a 5-8-membered heterocycle, and more preferably a 6-membered heterocycle; and
R⁵ is selected from C₁₋₆alkyl, C₁₋₆alkoxy, C₃₋₆cycloalkyl, C₃₋₆cycloalkyl-C₁₋₆alkyl, C₁₋₆alkoxyC₁₋₆alkyl, C₁₋₆alkylamino, diC₁₋₆alkylamino, and C₁₋₆alkylC₁₋₆alkoxyamino, wherein each of said groups is optionally substituted with one to three substituents independently selected from halogen and CN;
or a salt or N-oxide thereof;
wherein the compound of formula (I) is used as fungicide.

2. A compound of formula (I) 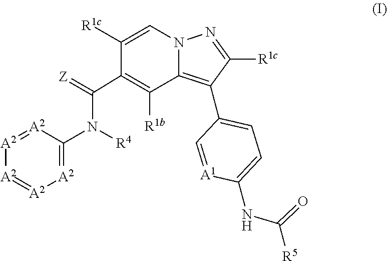
wherein Z is O or S, and preferably Z is O;
A¹ is CH or N, and preferably N;
R^{1a}, R^{1b} and R^{1c} are independently selected from hydrogen, hydroxy, halogen, CN, C₁₋₆alkyl, C₃₋₆-cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl;
A² are independently CR² or N, with the proviso that no more than three A² are N, preferably no more than two A² are N, preferably no more than one A² is N, and more preferably the four A² are CR²;
R² are independently selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl groups is optionally substituted with one to three substituents independently selected from halogen, hydroxy, and CN; with the proviso that at least two R² are not hydrogen;
A³ is independently CR³ or N;
R³ is selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, amino, C₁₋₆alkylamino, diC₁₋₆-alkylamino, and C₃₋₆cycloalkylamino, wherein each of the C₁₋₆alkyl, C₁₋₆alkoxy, C₁₋₆alkoxy-C₁₋₆alkyl, C₁₋₆alkoxy-C₁₋₆alkoxy, C₂₋₆alkenyl, C₂₋₆alkynyl, C₃₋₆cycloalkyl, C₃₋₆cycloalkyl-C₁₋₆alkyl, C₁₋₆alkylsulfanyl, C₁₋₆alkylsulfinyl, C₁₋₆alkylsulfonyl, amino, C₁₋₆alkylamino, diC₁₋₆-alkylamino, and C₃₋₆cycloalkylamino groups is optionally substituted with one to three substituents independently selected from halogen, hydroxy, and CN;
R⁴ is selected from C₁₋₆alkyl, C₁₋₆alkoxy-C₁₋₆alkyl, C₃₋₆cycloalkyl, C₃₋₆cycloalkyl-C₁₋₄alkyl, C₂₋₆ alkenyl, C₂₋₆alkynyl, C₁₋₆alkoxy, C₁₋₆alkylsulfanyl-C₁₋₆alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl, diC₁₋₆alkylaminocarbonyl-C₁₋₆alkyl, and CN, wherein each of the C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, C₁₋₆alkoxy, C₁₋₆alkylsulfanyl-C₁₋₆ alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl and diC₁₋₆alkylaminocarbonyl-C₁₋₆alkyl groups is optionally substituted with one to three substituents independently selected from halogen and CN;
wherein A³ and R⁴ taken together optionally form a ring, preferably a 5-8-membered heterocycle, and more preferably a 6-membered heterocycle; and
R⁵ is selected from C₁₋₆alkyl, C₁₋₆alkoxy, C₃₋₆cycloalkyl, C₃₋₆cycloalkyl-C₁₋₆alkyl, C₁₋₆alkoxyC₁₋₆alkyl, C₁₋₆alkylamino, diC₁₋₆alkylamino, and C₁₋₆alkylC₁₋₆alkoxyamino, wherein each of said groups is optionally substituted with one to three substituents independently selected from halogen and CN;
or a salt or N-oxide thereof.

3. The compound according to claim 1, wherein R^{1a}, R^{1b} and R^{1c} are independently selected from hydrogen, C₁₋₆alkyl, C₃₋₆cycloalkyl, C₁₋₆alkoxy-C₁₋₆alkyl, C₃₋₆cycloalkyl-C₁₋₄alkyl, and C₁₋₆alkoxy.

4. The compound according to claim 1, wherein R^{1a}, R^{1b} and R^{1c} are hydrogen.

5. The compound according to claim 1, wherein R³ is selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₃₋₆cycloalkyl-C₁₋₆alkyl, amino, C₁₋₆alkylamino, diC₁₋₆-alkylamino, and C₃₋₆cycloalkylamino, wherein each of the C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₃₋₆cycloalkyl-C₁₋₆alkyl, amino, C₁₋₆alkylamino, diC₁₋₆-alkylamino and C₃₋₆cycloalkylamino groups is optionally substituted with one to three substituents independently selected from halogen, hydroxy, and CN.

6. The compound according to claim 1, wherein four A² are CR² and A³ is N.

7. The compound according to claim 1, wherein 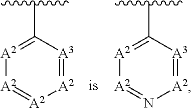
and preferably the three A² are CR² and A³ is CR³.

8. The compound according to claim 1, wherein 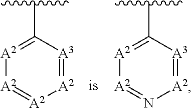
and preferably the three A² are CR² and A³ is CR³.

9. The compound according to claim 1, wherein four A² are CR² and A³ is CR³.

10. The compound according to claim 9, wherein

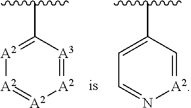

11. The compound according to claim 1, wherein R⁴ is selected from C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, and C₁₋₆alkoxy, wherein each of the C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, and C₁₋₆alkoxy groups is optionally substituted with one to three substituents independently selected from halogen and CN.

12. The compound according to claim 1, wherein R⁵ is selected from C₁₋₆alkyl, C₁₋₆alkoxy, C₃₋₆cycloalkyl, C₃₋₆cycloalkyl-C₁₋₆alkyl, and C₁₋₆alkoxyC₁₋₆alkyl, wherein each of said groups is optionally substituted with one to three substituents independently selected from halogen and CN.

13. A composition comprising a fungicidally effective amount of a compound as defined in claim 1.

14. A composition according to claim 13, wherein the composition further comprises at least one compound selected from an additional active ingredient, an appropriate formulation inert, a carrier, an adjuvant, and any mixtures thereof.

15. A method of combating, preventing or controlling phytopathogenic diseases which comprises applying to a phytopathogen, to the locus of a phytopathogen, to a plant susceptible to attack by a phytopathogen, or to a plant propagation material thereof, a fungicidally effective amount of a compound, or a composition comprising a compound according to claim 1.

16. A compound of formula (XI) 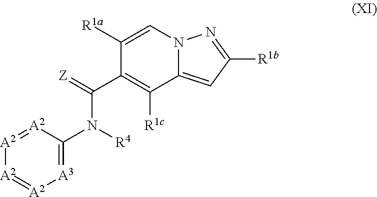
wherein Z is O or S, and preferably Z is O;
R^{1a}, R^{1b} and R^{1c} are independently selected from hydrogen, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl; and preferably R^{1a}, R^{1b} and R^{1c} are hydrogen;
A² are independently CR² or N, with the proviso that no more than three A² are N, preferably no more than two A² are N, preferably no more than one A² is N, and more preferably the four A² are CR²;
R² are independently selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl, wherein each of the C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl groups is optionally substituted with one substituent independently selected from halogen, hydroxy, and CN; with the proviso that at least two R² are preferably not hydrogen;
A³ is independently CR³ or N;
R³ is selected from hydrogen, hydroxy, halogen, CN, C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl, wherein each of the C₁₋₆alkyl, C₁₋₆alkoxy, C₁₋₆alkoxy-C₁₋₆alkyl, C₁₋₆alkoxy-C₁₋₆alkoxy, C₃₋₆cycloalkyl, C₁₋₆alkylsulfonyl, C₁₋₆alkoxycarbonyl, C₁₋₆alkylaminocarbonyl, diC₁₋₆alkylaminocarbonyl, and C₁₋₆alkylcarbonyl groups is optionally substituted with one substituent independently selected from halogen, hydroxy, and CN; and preferably R³ is hydrogen;
R⁴ is selected from C₁₋₆alkyl, C₁₋₆alkoxy-C₁₋₆alkyl, C₃₋₆cycloalkyl, C₃₋₆cycloalkyl-C₁₋₄alkyl, C₂₋₆ alkenyl, C₂₋₆alkynyl, C₁₋₆alkoxy, C₁₋₆alkylsulfanyl-C₁₋₆alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl, diC₁₋₆alkylaminocarbonyl-C₁₋₆alkyl, and CN, wherein each of the C₁₋₆alkyl, C₁₋₆alkoxy-C₁₋₆alkyl, C₃₋₆cycloalkyl, C₃₋₆cycloalkyl-C₁₋₄alkyl, C₂₋₆alkenyl, C₂₋₆alkynyl, C₁₋₆alkoxy, C₁₋₆alkylsulfanyl-C₁₋₆ alkyl, C₁₋₆alkylsulfinyl-C₁₋₆alkyl, C₁₋₆alkylsulfonyl-C₁₋₆alkyl, C₁₋₆alkoxycarbonyl-C₁₋₆alkyl, C₁₋₆alkylaminocarbonyl-C₁₋₆alkyl and diC₁₋₆alkylaminocarbonyl-C₁₋₆alkyl groups is optionally substituted with one to three substituents independently selected from halogen and CN; and
wherein A³ and R⁴ taken together optionally form a ring, preferably a 5-8-membered heterocycle, and more preferably a 6-membered heterocycle;
or a salt or N-oxide thereof.
